(19) United States
(12) Patent Application Publication
(10) Pub. No.: US 2023/0355678 A1
(43) Pub. Date: Nov. 9, 2023

(54) METHODS FOR IMPROVING T CELL EFFICACY

(71) Applicant: IMMATICS US, INC., Houston, TX (US)

(72) Inventors: Pooja MEHTA, Houston, TX (US); Rene TAVERA, Houston, TX (US); Christopher LANIER, Houston, TX (US); Mamta KALRA, Houston, TX (US)

(21) Appl. No.: 18/312,144

(22) Filed: May 4, 2023

Related U.S. Application Data
(60) Provisional application No. 63/338,788, filed on May 5, 2022.

(51) Int. Cl. A61K 35/17; C12N 5/0783

(52) U.S. Cl. CPC A61K 35/17; C12N 5/0636; C12N 2501/999

(57) ABSTRACT

Methods of manufacturing T cells to improve their efficacy, persistence, memory function, and/or antigen stimulated survival are provided. Methods of manufacturing T cells to improve production of Central Memory T (Tcm) cells are provided. Methods may include culturing or treating T cells with one or more histone deacetylase inhibitor (HDACi) and interleukin-21 (IL-21), with one or more kinase inhibitor, such a tyrosine kinase inhibitor, and/or with one or more AKT inhibitor (AKTi).

### RELATED APPLICATIONS

The present application is an U.S. Non-Provisional application claiming priority to U.S. Provisional Patent Application No. 63/338,788, filed on May 5, 2022, the entire contents of which are hereby incorporated by reference for all purposes.

### REFERENCE TO SEQUENCE LISTING SUBMITTED ELECTRONICALLY

The official copy of the sequence listing is submitted concurrently via EFS-Web as an ASCII-formatted sequence listing with a file named “3000011-028001_Sequence-LIsting_S T26” created on May 2, 2023, and having a size of 396,665 bytes, and is filed concurrently with the specification. The sequence listing contained in this ASCII-formatted document is part of the specification and is herein incorporated by reference in its entirety.

### BACKGROUND

### Field

The present disclosure relates to methods for improving efficacy of T cells for use in immunotherapy. The invention further relates to methods for T cell manufacturing that provide improved T cell activation and result in a T cell population with improved efficacy, persistence, memory function, and antigen stimulated survival.

### Background

T cell exhaustion describes a state in which T cells progressively decrease and finally cease to proliferate and function due to excessive antigenic stimulation in the absence of co-stimulation. T cell exhaustion is often found in chronic infection and cancer. Terminally differentiated and functionally exhausted T cells are associated with a poor clinical response.

It is desirable to develop methods of manufacturing T cells with less differentiated T cell phenotype for immunotherapy.

### BRIEF SUMMARY

In an aspect, the present disclosure may be related to a method of manufacturing modified T cells including: activating a population of T cells, transducing the activated T cells with a viral vector, expanding the transduced T cells, in which the activating, the transducing, and/or the expanding may be performed in the presence of a histone deacetylase inhibitor (HDACi), and obtaining the expanded T cells.

In some aspects, the activating may be performed in the presence of the HDACi and the transducing and the expanding may be performed in the absence of the HDACi.

In some aspects, the activating and the transducing may be performed in the presence of the HDACi and the expanding may be performed in the absence of the HDACi.

In some aspects, the activating and the expanding may be performed in the presence of the HDACi and the transducing may be performed in the absence of the HDACi.

In some aspects, the transducing and the expanding may be performed in the presence of the HDACi and the activating may be performed in the absence of the HDACi.

In some aspects, the activating, the transducing, and the expanding may be performed in the presence of the HDACi.

In some aspects, the HDACi may be selected from the group consisting of vorinostat (SAHA), belinostat, panobinostat, dacinostat, entinostat, tacedinaline, mocetinostat, and any combination thereof.

In an aspect, the present disclosure may be related to a method of manufacturing modified T cells including: activating a population of T cells, transducing the activated T cells with a viral vector, expanding the transduced T cells, in which the activating, the transducing, and/or the expanding may be performed in the presence of an AKT inhibitor (AKTi), and obtaining the expanded T cells.

In some aspects, the activating may be performed in the presence of the AKTi and the transducing and the expanding may be performed in the absence of the AKTi.

In some aspects, the activating and the transducing may be performed in the presence of the AKTi and the expanding may be performed in the absence of the AKTi.

In some aspects, the activating and the expanding may be performed in the presence of the AKTi and the transducing may be performed in the absence of the AKTi.

In some aspects, the transducing and the expanding may be performed in the presence of the AKTi and the activating may be performed in the absence of the AKTi.

In some aspects, the activating, the transducing, and the expanding may be performed in the presence of the AKTi.

In some aspects, the AKTi may be selected from the group consisting of (i) 3-[1-[[4-(7-phenyl-3H-imidazo[4, 5g]quinoxalin-6-yl)phenyl[methyl[piperidin-4-yl]-1H-benzimidazol-2-one; (ii) N,N dimethyl-1-[4-(6-phenyl-1H-imidazo[4, 5-g]quinoxalin-7-yl)phenyl[metha-namine; and (iii) I-(I-[4-(3-phenylbenzo[g]quinoxalin-2-yl)benzyl[piperidin-4-yl)-1,-3-dihy-dro-2H benzimidazol-2-one; A6730, B2311, 124018, GSK2110183 (afuresertib), Perifosine (KRX-0401), GDC-0068 (ipatasertib), RX-0201, VQD-002, LY294002, A-443654, A-674563, Akti-1, Akti-2, Akti-1/2, AR-42, API-59CJ-OMe, ATI-13148, AZD-5363, erucylphosphocholine, GSK-2141795 (GSK795), KP372-1, L-418, L-71-101, PBI-05204, PIA5, PX-316, SR13668, triciribine, GSK 690693 (CAS #937174-76-0), FPA 124 (CAS #902779-59-3), Miltefosine, PHT-427 (CAS #1 191951-57-1), 10-DEBC hydrochloride, Akt inhibitor III, MK-2206 dihydrochloride (CAS #1032350-13-2), SC79, AT7867 (CAS #857531-00-1), CCT128930 (CAS #885499-61-6), A-674563 (CAS #552325-73-2), AGL 2263, AS-041 164 (5-benzo[1,3]dioxol-5-ylmethylene-thiazolidine-2,4-dione), BML-257 (CAS #32387-96-5), XL-418, CAS #612847-09-3, CAS #98510-80-6, H-89 (CAS #127243-85-0), OXY-1 1 1 A, 3-[1-[[4-(7-phenyl-3H-imidazo[4,5-g]quinoxalin-6-yl)phenyl]methyl]piperid-in-4-yl]-1H-benzimidazol-2-one, N,N-dimethyl-1-[4-(6-phenyl-1H-imidazo[4,5-g]quinoxalin-7-yl)phenyl]metha-namine, 1-{1-[[4-(3-phenylbenzo[g]quinoxalin-2-yl)benzyl]piperidin-4-yl}-1-,-3-dihydro-2H-benzimidazol-2-one, and any combination thereof.

In an aspect, the present disclosure may be related to a method of manufacturing modified T cells including: activating a population of T cells, transducing the activated T cells with a viral vector, expanding the transduced T cells, in which the activating, the transducing, and/or the expanding may be performed in the presence of a tyrosine kinase inhibitor (TKi), and obtaining the expanded T cells.

In some aspects, the activating may be performed in the presence of the TKi and the transducing and the expanding may be performed in the absence of the TKi.

In some aspects, the activating and the transducing may be performed in the presence of the TKi and the expanding may be performed in the absence of the TKi.

In some aspects, the activating and the expanding may be performed in the presence of the TKi and the transducing may be performed in the absence of the TKi.

In some aspects, the transducing and the expanding may be performed in the presence of the TKi and the activating may be performed in the absence of the TKi.

In some aspects, the activating, the transducing, and the expanding may be performed in the presence of the TKi.

In some aspects, the TKi may be selected from the group consisting of dasatinib, saracatinib, bosutinib, nilotinib, PP1-inhibitor, and any combination thereof.

In some aspects, the activating, the transducing, and/or the expanding may be further performed in the presence of at least one cytokine.

In some aspects, the at least one cytokine may be selected from the group consisting of interleukin (IL)-2, IL-7, IL-12, IL-15, IL-18, and IL-21.

In some aspects, the viral vector may be a retroviral vector or a lentiviral vector.

In some aspects, the viral vector may encode a TCR and/or a CAR.

In some aspects, the T cells may be CD4+ T cells.

In some aspects, the T cells may be CD8+ T cells.

In some aspects, the T cells may be γδ T cells or αβ T cells.

In some aspects, the activating may include contacting the T cells with an anti-CD3 antibody and an anti-CD28 antibody.

In some aspects, the present disclosure may be related to a population of T cells obtained from the method of the present disclosure.

In some aspects, the present disclosure may be related to a composition containing the population of T cells obtained from the method of the present disclosure.

In some aspects, the composition may further contain an adjuvant selected from the group consisting of an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), and combinations thereof.

In some aspects, the present disclosure may be related to a method of treating a patient who has cancer, including administering to the patient the composition of the present disclosure, in which the cancer may be selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

In some aspects, the present disclosure may be related to a method of eliciting an immune response in a patient who has cancer, including administering to the patient the composition of the present disclosure, in which the cancer may be selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

In some aspects, the concentration of the HDACi may be from about 0.1 nM to about 5 mM, from about 0.1 nM to about 4 mM, from about 0.1 nM to about 3 mM, from about 0.1 nM to about 2 mM, from about 0.1 nM to about 1 mM, from about 0.1 nM to about 900 nM, from about 0.1 nM to about 800 nM, from about 0.1 nM to about 700 nM, from about 0.1 nM to about 600 nM, from about 0.1 nM to about 500 nM, from about 0.1 nM to about 400 nM, from about 0.1 nM to about 300 nM, from about 0.1 nM to about 200 nM, from about 0.1 nM to about 100 nM, from about 0.1 nM to about 50 nM, from about 0.1 nM to about 40 nM, from about 0.1 nM to about 30 nM, from about 0.1 nM to about 20 nM, from about 0.1 nM to about 10 nM, from about 0.1 nM to about 5 nM, from about 0.1 nM to about 4 nM, from about 0.1 nM to about 3 nM, from about 0.1 nM to about 2 nM, from about 0.1 nM to about 1 nM, from about 0.2 nM to about 1 nM, from about 0.3 nM to about 1 nM, from about 0.4 nM to about 1 nM, from about 0.5 nM to about 1 nM, from about 0.5 nM to about 0.9 nM, from about 0.5 nM to about 0.8 nM, from about 0.5 nM to about 0.7 nM, from about 0.5 nM to about 0.6 nM, from about 1 nM to about 50 nM, or from about 2 nM to about 25 nM.

In some aspects, the concentration of the AKTi may be from about 1 nM to about 1 mM, from about 10 nM to about 1 mM, from about 100 nM to about 1 mM, from about 100 nM to about 500 μM, from about 100 nM to about 100 μM, from about 100 nM to about 50 μM, from about 100 nM to about 10 μM, from about 100 nM to about 1 μM, from about 100 nM to about 900 nM, from about 100 nM to about 800 nM, from about 100 nM to about 700 nM, from about 100 nM to about 600 nM, from about 100 nM to about 500 nM, from about 100 nM to about 400 nM, from about 100 nM to about 300 nM, from about 150 nM to about 300 nM, from about 200 nM to about 300 nM, from about 250 nM to about 300 nM, from about 1 μM to about 1 mM, from about 10 μM to about 1 mM, from about 100 μM to about 1 mM, from about 1 nM to about 100 μM, from about 1 nM to about 10 μM, from about 1 nM to about 1 μM, from about 1 nM to about 100 nM, from about 1 nM to about 50 nM, from about 100 nM to about 100 μM, from about 500 nM to about 50 μM, from about 1 μM to about 50 μM, from about 1 μM to about 10 μM, or from about 5 μM to about 10 μM.

In some aspects, the concentration of the TKi may be from about 1 nM to about 1 μM, from about 1 nM to about 500 nM, from about 1 nM to about 400 nM, from about 1 nM to about 300 nM, from about 1 nM to about 200 nM, from about 1 nM to about 150 nM, from about 1 nM to about 100 nM, from about 1 nM to about 50 nM, from about 1 nM to about 40 nM, from about 1 nM to about 30 nM, from about 1 nM to about 20 nM, from about 1 nM to about 10 nM, from about 2 nM to about 10 nM, from about 3 nM to about 10 nM, from about 4 nM to about 10 nM, from about 5 nM to about 10 nM, from about 100 nM to about 1 mM, from about 100 nM to about 500 μM, from about 100 nM to about 100 μM, from about 100 nM to about 50 μM, from about 100 nM to about 10 μM, from about 100 nM to about 1 μM, from about 100 nM to about 900 nM, from about 100 nM to about 800 nM, from about 100 nM to about 700 nM, from about 100 nM to about 600 nM, from about 100 nM to about 500 nM, from about 100 nM to about 400 nM, from about 100 nM to about 300 nM, from about 150 nM to about 300 nM, from about 200 nM to about 300 nM, from about 250 nM to about 300 nM, from about 1 μM to about 1 mM, from about 10 μM to about 1 mM, from about 100 μM to about 1 mM, from about 1 nM to about 100 μM, from about 1 nM to about 10 μM, from about 1 nM to about 1 μM, from about 1 nM to about 100 nM, from about 1 nM to about 50 nM, from about 100 nM to about 100 μM, from about 500 nM to about 50 μM, from about 1 μM to about 50 μM, from about 1 μM to about 10 μM, or from about 5 μM to about 10 μM

In some aspects, the activating may be carried out within a period of from about 1 hour to about 120 hours, about 1 hour to about 108 hours, about 1 hour to about 96 hours, about 1 hour to about 84 hours, about 1 hour to about 72 hours, about 1 hour to about 60 hours, about 1 hour to about 48 hours, about 1 hour to about 36 hours, about 1 hour to about 24 hours, about 2 hours to about 24 hours, about 4 hours to about 24 hours, about 6 hours to about 24 hours, about 8 hours to about 24 hours, about 10 hours to about 24 hours, about 12 hours to about 24 hours, about 12 hours to about 72 hours, about 24 hours to about 72 hours, about 6 hours to about 48 hours, about 24 hours to about 48 hours, about 6 hours to about 72 hours, or about 1 hours to about 12 hours.

In some aspects, the transducing may be carried out within a period of from about 1 hour to 120 hours, about 1 hour to 108 hours, about 1 hour to 96 hours, about 1 hour to 72 hours, about 1 hour to 48 hours, about 1 hour to 36 hours, about 1 hour to 24 hours, about 2 hour to 24 hours, about 4 hour to 24 hours, about 6 hour to 24 hours, about 8 hour to 24 hours, about 10 hour to 24 hours, about 12 hour to 24 hours, about 14 hour to 24 hours, about 16 hour to 24 hours, about 18 hour to 24 hours, about 20 hour to 24 hours, or about 22 hour to 24 hours.

In some aspects, the expanding may be carried out within a period of from about 1 day to about 30 days, about 1 day to about 25 days, about 1 day to about 20 days, about 1 day to about 15 days, about 1 day to about 10 days, about 2 days to about 10 days, about 3 days to about 10 days, about 4 days to about 10 days, about 5 days to about 10 days, about 6 days to about 10 days, about 7 days to about 10 days, about 8 days to about 10 days, or about 9 days to about 10 days.

### BRIEF DESCRIPTION OF THE DRAWINGS

FIG. 1 shows an experimental design according to the present disclosure.

FIG. 2A shows viability of T cell products prepared by HDACi treatment according to the present disclosure.

FIG. 2B shows fold expansion of T cell products prepared by HDACi treatment according to the present disclosure.

FIG. 3 shows transduction efficiency of T cell products prepared by HDACi treatment according to the present disclosure.

FIG. 4 shows phenotypes of T cell products prepared by HDACi treatment according to the present disclosure.

FIG. 5 shows phenotypes of T cell products prepared by HDACi treatment according to the present disclosure.

FIG. 6 shows phenotypes of T cell products prepared by HDACi treatment according to the present disclosure.

FIG. 7A shows phenotypes of T cell products prepared by HDACi treatment according to the present disclosure.

FIG. 7B shows phenotypes of T cell products prepared by HDACi treatment according to the present disclosure.

FIG. 8 shows phenotypes of T cell products prepared by HDACi treatment according to the present disclosure.

FIG. 9 shows an experimental design according to the present disclosure.

FIG. 10A shows fold expansion of T cell products prepared by HDACi±IL-21 treatment according to the present disclosure.

FIG. 10B shows viability of T cell products prepared by HDACi±IL-21 treatment according to the present disclosure.

FIG. 11 shows transduction efficiency of T cell products prepared by HDACi±IL-21 treatment according to the present disclosure.

FIG. 12A shows phenotypes of T cell products prepared by HDACi treatment according to the present disclosure.

FIG. 12B shows phenotypes of T cell products prepared by HDACi treatment according to the present disclosure.

FIG. 12C shows phenotypes of T cell products prepared by HDACi treatment according to the present disclosure.

FIG. 13 shows phenotypes of T cell products prepared by HDACi treatment according to the present disclosure.

FIG. 14A shows phenotypes of T cell products prepared by HDACi treatment according to the present disclosure.

FIG. 14B shows phenotypes of T cell products prepared by HDACi treatment according to the present disclosure.

FIG. 15A shows phenotypes of T cell products prepared by HDACi treatment according to the present disclosure.

FIG. 15B shows phenotypes of T cell products prepared by HDACi treatment according to the present disclosure.

FIG. 16A shows phenotypes of T cell products prepared by HDACi treatment according to the present disclosure.

FIG. 16B shows phenotypes of T cell products prepared by HDACi treatment according to the present disclosure.

FIG. 17A shows phenotypes of T cell products prepared by HDACi±IL-21 treatment according to the present disclosure.

FIG. 17B shows phenotypes of T cell products prepared by HDACi±IL-21 treatment according to the present disclosure.

FIG. 18 shows an experimental design according to the present disclosure.

FIG. 19A shows fold expansion of T cell products prepared by HDACi±IL-21 or AKT inhibitor (AKTi) treatment according to the present disclosure.

FIG. 19B shows transduction efficiency of T cell products prepared by HDACi±IL-21 or AKT inhibitor (AKTi) treatment according to the present disclosure.

FIG. 19C shows transduction efficiency of T cell products prepared by HDACi±IL-21 or AKT inhibitor (AKTi) treatment according to the present disclosure.

FIG. 20A shows phenotypes of T cell products prepared by HDACi±IL-21 or AKT inhibitor (AKTi) treatment according to the present disclosure.

FIG. 20B shows phenotypes of T cell products prepared by HDACi±IL-21 or AKT inhibitor (AKTi) treatment according to the present disclosure.

FIG. 20C shows phenotypes of T cell products prepared by HDACi±IL-21 or AKTi treatment according to the present disclosure.

FIG. 21A shows tumor cell killing of T cell products prepared by HDACi±IL-21 or AKTi treatment according to the present disclosure.

FIG. 21B shows tumor cell killing of T cell products prepared by HDACi±IL-21 or AKTi treatment according to the present disclosure.

FIG. 22A shows fold expansion of T cell products prepared by tyrosine kinase inhibitor (TKi) treatment according to the present disclosure.

FIG. 22B shows transduction efficiency of T cell products prepared by TKi treatment according to the present disclosure.

FIG. 22C shows transduction efficiency of T cell products prepared by TKi treatment according to the present disclosure.

FIG. 23A shows phenotypes of T cell products prepared by TKi treatment according to the present disclosure.

FIG. 23B shows phenotypes of T cell products prepared by TKi treatment according to the present disclosure.

FIG. 23C shows phenotypes of T cell products prepared by TKi treatment according to the present disclosure.

FIG. 24A shows fold expansion of T cell products prepared by HDACi±IL-21 or AKTi or TKi treatment according to the present disclosure.

FIG. 24B shows transduction efficiency of T cell products prepared by HDACi±IL-21 or AKTi or TKi treatment according to the present disclosure.

FIG. 24C shows transduction efficiency of T cell products prepared by HDACi±IL-21 or AKTi or TKi treatment according to the present disclosure.

FIG. 25A shows phenotypes of T cell products prepared by HDACi±IL-21 or AKTi or TKi treatment according to the present disclosure.

FIG. 25B shows phenotypes of T cell products prepared by HDACi±IL-21 or AKTi or TKi treatment according to the present disclosure.

FIG. 26A shows phenotypes of T cell products prepared by HDACi±IL-21 or AKTi or TKi treatment according to the present disclosure.

FIG. 26B shows phenotypes of T cell products prepared by HDACi±IL-21 or AKTi or TKi treatment according to the present disclosure.

FIG. 27A shows phenotypes of T cell products prepared by HDACi or AKTi or TKi treatment according to the present disclosure.

FIG. 27B shows phenotypes of T cell products prepared by HDACi or AKTi or TKi treatment according to the present disclosure.

FIG. 28A shows tumor cell killing of T cell products prepared by HDACi or AKTi or TKi treatment according to the present disclosure.

FIG. 28B shows tumor cell killing of T cell products prepared by HDACi or AKTi or TKi treatment according to the present disclosure.

FIG. 29A shows tumor cell killing of T cell products prepared by AKTi or TKi treatment according to the present disclosure.

FIG. 29B shows tumor cell killing of T cell products prepared by AKTi or TKi treatment according to the present disclosure.

FIG. 29C shows tumor cell killing of T cell products prepared by AKTi or TKi treatment according to the present disclosure.

### DETAILED DESCRIPTION

### T Cell Exhaustion

Exhaustion may be a hallmark of, and obstacle to, many cell-based immunotherapies. Exhaustion may be the decreased functionality and effectiveness of an immune effector cell's response to specific antigen. In individuals with cancer or chronic viral infections, antigen specific T cells may be generally present, yet when exhausted, lack the ability to proliferate, secrete helper cytokines/chemokines, and/or kill target cells that display antigen. Exhaustion affects both CD4+ and CD8+ T cells. Other cells that are deployed in cell based therapies, such as NK cells, can exhibit signs of exhaustion marked by decreases in cytokine secretion and target cell killing.

T cell exhaustion can be characterized by the inability to express cytokines and effector molecules, as well as by the increased expression of inhibitory receptors, both of which may be the consequence of epigenetic reprogramming Inhibitory receptors may include, e.g., CTLA-4, LAG-3, PD-1, and TIM-3. For example, the prototypic exhaustion marker, programmed death 1 (PD-1), may be strongly expressed by exhausted T cells in a process mediated by alteration of epigenetic marks and open chromatin regions. These epigenetic changes may enforce PD-1 expression and curb T-cell effector functions. PD-1 expression can thus constitute an effective physiological mechanism to maintain T cells in the repertoire, preventing continued division so that T cells may not reach the Hayflick limit (the number of times a normal somatic, differentiated human cell population can divide before cell division stops) and undergo senescence.

The sustained expression of multiple inhibitory receptors may be the hallmark of exhausted T cells. Common to both CD4+ and CD8+ exhausted T cells may be the surface expression of multiple inhibitory receptors, e.g., PD-1, CTLA-4, LAG-3, TIM3, CD244, CD160 and TIGIT, and others. Flow cytometry can be used to phenotype both the surface markers and intracellular markers of exhausted T cells. Exhausted T cells may have decreased intracellular TNFα and INFγ. Exhausted CD4+ T cells may become skewed towards a Tfh (T follicular helper cell) phenotype and express the surface markers CXCR5 and ICOS. Normal CD8+ effector T cells may co-express the transcription factors T-bet (T-box transcription factor), and EOMES (eomesodermin), while exhausted T cells may express one or the other of these factors. Two exhausted CD8+ T cell populations may be defined as follows: The first may be EOMES^{high} PD-1^{high}, TIM3⁺ and CD160⁺, has high granzyme expression, but limited proliferation capability. The second population may be T-bet^{high} PD-1^{low}, has low proliferative capacity, and can still secrete TNFα and INFγ.

T cell exhaustion, however, may be a transient state that can be reversed by the activation of certain signaling pathways. For example, checkpoint blockade inhibitors, e.g., blocking antibodies against these inhibitory receptors, have shown remarkable efficacy in reversing exhaustion and promoting tumor regression. Reviving exhausted T cells may holds much therapeutic promise. Targeting PD-1 and other over-expressed inhibitory markers may be a good strategy to reverse exhaustion. A combined blockade of PD1-B7-H1 (PDL-1), with other co-inhibitors, most notably inhibitors of TIM3, CTLA-4 and LAGS, may have a synergistic effect in reversing exhaustion. The combination of IL-2 with PD-1 inhibition during chronic infection may invigorate exhausted T cells. Also, an agonistic monoclonal antibody specific for 4-1BB (CD137) in combination with IL-7 may restore the activity of dysfunctional CD8+ T cells in the lymphocytic choriomeningitis virus (LCMV) mouse model.

### Histone Deacetylase Inhibitor (HDACi)

As used herein, the terms “histone deacetylase”, “HDAC enzyme”, or “HDAC” refer to a class of enzymes (EC 3.5.1.98) that catalyze removal of acetyl groups (CH3-CO—R) from, for example, e-N-acetyl-lysine amino acids on a histone. Histone acetylation and de-acetylation plays an important role in regulating gene expression. The acetylation of histones is thought to neutralize their positive charges and loosen their interaction with negatively-charged DNA. This opens the chromatin structure to facilitate the binding of transcription factors and, subsequently, gene transcription. De-acetylation of histones by HDACs tightens their interaction with DNA, resulting in a closed chromatin structure and the inhibition of gene transcription. Histone lysine acetylation is highly reversible. A lysine residue becomes acetylated by the action of the histone/lysine acetyltransferase enzymes (HATs), and de-acetylated by histone deacetylases (HDACs).

As used herein, the term “HDAC inhibitor” or “HDACi” refers to a class of compounds capable of potently and specifically inhibiting the histone deacetylase activity of one or more HDAC enzymes. Classical HDACis act on conventional HDACs in Classes I, II, and IV, comprising those HDACs requiring Zn2+ as a cofactor for their deacetylase activity. Classical HDACi are typically grouped according to the chemical moiety responsible for binding to the zinc ion, except for cyclic tetrapeptides, which bind to the zinc ion with a thiol group. Exemplary classical HDACis comprise hydroxamic acids or hydroxamates (e.g., trichostatin A [CAS No. 58880-19-6]), cyclic tetrapeptides (e.g., trapoxin B [CAS No. 133155-90-5]) and depsipeptides, benzamides, electrophilic ketones, and aliphatic acids (e.g., phenylbutyrate and valproic acid). Second generation classical HDACis include the hydroxamic acids vorinostat (suberanilohydroxamic acid or SAHA, marketed as Zolinza® [CAS No. 149647-78-9]), belinostat (PXD101, marketed as Beleodaq® [CAS No. 866323-14-0]), panobinostat (marketed as Farydaq® [CAS No. 404950-80-7]), and dacinostat (LAQ824) [DAS 404951-53-7], and the benzamides entinostat (SNDX-275 or MS-275) [CAS No. 209783-80-2], tacedinaline (CI994) [CAS No. 112522-64-2], and mocetinostat (MGCD0103) [CAS No. 726169-73-9].

The present methods may further concern the treatment of effector T cells with IL-21 in combination with HDACi for improving T cell efficacy, persistence, memory function, and/or antigen stimulated survival. The present methods may further concern the treatment of effector T cells with IL-21 in combination with HDACi for the improved production of Central Memory T (Tcm) cells. The starting population of lymphocytes may be cultured sequentially with an HDACi and IL-21.

The starting population of lymphocytes may be cultured with an HDACi (for example, from about 0.1 nM to about 5 mM, from about 0.1 nM to about 4 mM, from about 0.1 nM to about 3 mM, from about 0.1 nM to about 2 mM, from about 0.1 nM to about 1 mM, from about 0.1 nM to about 900 nM, from about 0.1 nM to about 800 nM, from about 0.1 nM to about 700 nM, from about 0.1 nM to about 600 nM, from about 0.1 nM to about 500 nM, from about 0.1 nM to about 400 nM, from about 0.1 nM to about 300 nM, from about 0.1 nM to about 200 nM, from about 0.1 nM to about 100 nM, from about 0.1 nM to about 50 nM, from about 0.1 nM to about 40 nM, from about 0.1 nM to about 30 nM, from about 0.1 nM to about 20 nM, from about 0.1 nM to about 10 nM, from about 0.1 nM to about 5 nM, from about 0.1 nM to about 4 nM, from about 0.1 nM to about 3 nM, from about 0.1 nM to about 2 nM, from about 0.1 nM to about 1 nM, from about 0.2 nM to about 1 nM, from about 0.3 nM to about 1 nM, from about 0.4 nM to about 1 nM, from about 0.5 nM to about 1 nM, from about 0.5 nM to about 0.9 nM, from about 0.5 nM to about 0.8 nM, from about 0.5 nM to about 0.7 nM, from about 0.5 nM to about 0.6 nM, from about 1 nM to about 50 nM, or from about 2 nM to about 25 nM) and then cultured with IL-21 (for example, from about 1 ng/ml to about 100 ng/ml, from about 5 ng/ml to about 90 ng/ml, from about 10 ng/ml to about 80 ng/ml, from about 10 ng/ml to about 70 ng/ml, from about 10 ng/ml to about 60 ng/ml, from about 10 ng/ml to about 50 ng/ml, from about 15 ng/ml to about 45 ng/ml, from about 20 ng/ml to about 40 ng/ml, from about 25 ng/ml to about 35 ng/ml, from about 25 ng/ml to about 30 ng/ml, about 30 ng/ml, or about 35 ng/ml).

The starting population of lymphocytes may be cultured with IL-21 (for example, from about 1 ng/ml to about 100 ng/ml, from about 5 ng/ml to about 90 ng/ml, from about 10 ng/ml to about 80 ng/ml, from about 10 ng/ml to about 70 ng/ml, from about 10 ng/ml to about 60 ng/ml, from about 10 ng/ml to about 50 ng/ml, from about 15 ng/ml to about 45 ng/ml, from about 20 ng/ml to about 40 ng/ml, from about 25 ng/ml to about 35 ng/ml, from about 25 ng/ml to about 30 ng/ml, about 30 ng/ml, or about 35 ng/ml) and then cultured with an HDACi (for example, from about 0.1 nM to about 5 mM, from about 0.1 nM to about 4 mM, from about 0.1 nM to about 3 mM, from about 0.1 nM to about 2 mM, from about 0.1 nM to about 1 mM, from about 0.1 nM to about 900 nM, from about 0.1 nM to about 800 nM, from about 0.1 nM to about 700 nM, from about 0.1 nM to about 600 nM, from about 0.1 nM to about 500 nM, from about 0.1 nM to about 400 nM, from about 0.1 nM to about 300 nM, from about 0.1 nM to about 200 nM, from about 0.1 nM to about 100 nM, from about 0.1 nM to about 50 nM, from about 0.1 nM to about 40 nM, from about 0.1 nM to about 30 nM, from about 0.1 nM to about 20 nM, from about 0.1 nM to about 10 nM, from about 0.1 nM to about 5 nM, from about 0.1 nM to about 4 nM, from about 0.1 nM to about 3 nM, from about 0.1 nM to about 2 nM, from about 0.1 nM to about 1 nM, from about 0.2 nM to about 1 nM, from about 0.3 nM to about 1 nM, from about 0.4 nM to about 1 nM, from about 0.5 nM to about 1 nM, from about 0.5 nM to about 0.9 nM, from about 0.5 nM to about 0.8 nM, from about 0.5 nM to about 0.7 nM, from about 0.5 nM to about 0.6 nM, from about 1 nM to about 50 nM, or from about 2 nM to about 25 nM).

In embodiments, the starting population of lymphocytes may be cultured simultaneously in the presence of an HDACi and IL-21. In embodiments, the starting population of lymphocytes may be cultured simultaneously in the presence of an HDACi and IL-21 for a period of time sufficient to induce a Tcm phenotype. In embodiments, the starting population of lymphocytes may be cultured simultaneously in the presence of an HDACi and IL-21 for from 7 to 20 days. In embodiments, the starting population of lymphocytes may be cultured simultaneously in the presence of an HDACi and IL-21 for from 12 to 16 days. In certain embodiments, the starting population of lymphocytes may be cultured simultaneously in the presence of an HDACi and IL-21 for about 7, 8, 9, 10, 11, 12, 13, 14, 15, 16, 17, 18, 19, or 20 days. In embodiments, the starting population of lymphocytes may be cultured simultaneously in the presence of an HDACi and IL-21 for about 13, 14, or 15 days.

In embodiments, the starting population of lymphocytes may be further cultured in the presence of one or more additional cytokines, chemokines, or growth factors. In embodiments, the starting population of lymphocytes may be further cultured in the presence of IL-2. In certain embodiments, the starting population of lymphocytes may be cultured in the presence of IL-2 prior to being cultured simultaneously in the presence of an HDACi and IL-21.

In embodiments, the HDACi comprises a classical HDACi requiring Zn2+ as a cofactor for its deacetylase activity. In embodiments, the classical HDACi is selected from the group consisting of hydroxamic acids or hydroxamates, cyclic tetrapeptides and depsipeptides, benzamides, electrophilic ketones, and aliphatic acids. In embodiments, the HDACi comprises a hydroxamic acid or hydroxamate. In embodiments, the hydroxamic acid or hydroxamate is selected from the group consisting of vorinostat (suberanilohydroxamic acid or SAHA, marketed as Zolinza®), belinostat (PXD101, marketed as Beleodaq®), panobinostat (marketed as Farydaq®), and dacinostat (LAQ824). In embodiments, the HDACi comprises a benzamide. In embodiments, the benzamide is selected from the group consisting of entinostat (SNDX-275 or MS-275), tacedinaline (CI994), and mocetinostat (MGCD0103). In certain embodiments, the HDACi comprises a cyclic tetrapeptide or depsipeptides. In embodiments, the cyclic tetrapeptide or depsipeptide is trapoxin B. In embodiments, the HDACi may be an aliphatic acid. In embodiments, the aliphatic acid is selected from the group consisting of phenylbutyrate and valproic acid.

### Interleukin-21 (IL-21)

Human Interleukin 21 (IL-21) is a protein cytokine encoded by the IL-21 gene that has potent regulatory effects on cells of the immune system, including natural killer (NK) cells and cytotoxic T cells that can destroy virally infected or cancerous cells. The 162 amino acid human IL-21 protein (GenBank Accession No. BBA22643; SEQ ID NO: 1) is described in U.S. Pat. Nos. 6,307,024, and 6,686,178, both of which are incorporated herein by reference in their entireties. In embodiments, the present methods concern the treatment of effector T cells with IL-21 in combination with HDACi for improving T cell efficacy, persistence, memory function, and/or antigen stimulated survival. In embodiments, the present methods concern the treatment of effector T cells with IL-21 in combination with HDACi for the improved production of Central Memory T (Tcm) cells. In some aspects, the IL-21 is present in the culture media at a concentration of about 10 ng/mL to 50 ng/mL, about 15 ng/mL to 60 mg/mL, about 20 ng/mL to 40 ng/mL, or about 25, about 30, or about 35 ng/mL.

### AKT Inhibitor (AKTi)

“AKT inhibitor”, “AKTI”, or “AKTi” can be used interchangeably and refer to any molecule (e.g., AKT antagonist), including, but not limited to a small molecule, a polynucleotide (e.g., DNA or RNA), or a polypeptide (e.g., an antibody or an antigen-binding portion thereof), capable of blocking, reducing, or inhibiting the activity of AKT. AKT is a serine/threonine kinase, also known as protein kinase B or PKB. An AKT inhibitor can act directly on AKT, e.g., by binding AKT, or it can act indirectly, e.g., by interfering with the interaction between AKT and a binding partner or by inhibiting the activity of another member of the PI3K-AKT-mTOR pathway. Non-limiting examples of AKTi are shown in US20200206265, the content of which is hereby incorporated by reference in its entirety.

In embodiments, the present methods concern the treatment of effector T cells with AKTi for improving T cell efficacy, persistence, memory function, and/or antigen stimulated survival. In embodiments, the present methods concern the treatment of effector T cells with AKTi for the improved production of Central Memory T (Tcm) cells. In certain embodiments, the AKT inhibitor is a compound selected from the group consisting of:
(a) 3-[1-[[4-(7-phenyl-3H-imidazo[4, 5g]quinoxalin-6-yl)phenyl]methyl]piperidin-4-yl]-1H-benzimidazol-2-one [CAS No. 612847-09-3];
(b) N,N-dimethyl-1-[4-(6-phenyl-1H-imidazo[4,5-g]quinoxalin-7-yl)phenyl]methanamine,

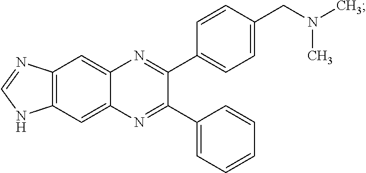 [SMILES: CN(C)Cc1ccc(-c2nc3cc4nc[nH]c4cc3nc2-c2ccccc2)cc1]

(c) 1-(1-[4-(3-phenylbenzo[g]quinoxalin-2-yl)benzyl]piperidin-4-yl)-1,3-dihydro-2H-benzimidazol-2-one;
(d) 3-[1-[[4-(7-phenyl-3H-imidazo[4,5-g]-15-uinoxaline-6-yl)phenyl]methyl]piperidin-4-yl]-1H-benzimidazol-2-one (A6730) [CAS No. 612847-09-3];
(e) 6-benzothiazol-2-yl-1-ethyl-2-[2-(methyl-phenyl-amino)-vinyl]-3-phenyl-3H-benzoimidazol-1-ium (B2311),

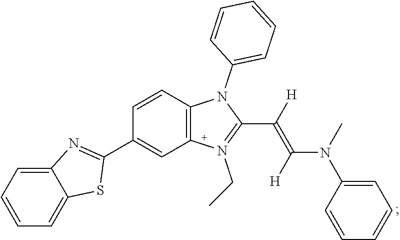

(f) GSK2110183 (afuresertib) [CAS No. 1047644-62-1];
(g) Perifosine (KRX-0401) [CAS No. 157716-52-4];
(h) GDC-0068 (ipatasertib) [CAS No. 1001264-89-6];
(i) 2′-deoxy-P-thio-guanylyl-(3′->5′)-2′-deoxy-P-thio-cytidylyl-(3′->5′)-P-thio-thymidylyl-(3′->5′)-2′-deoxy-P-thio-guanylyl-(3′->5′)-2′-deoxy-P-thio-cytidylyl-(3′->5′)-2′-deoxy-P-thio-adenylyl-(3′->5′)-P-thio-thymidylyl-(3′->5′)-2′-deoxy-P-thio-guanylyl-(3′->5′)-2′-deoxy-P-thio-adenylyl-(3′->5′)-P-thio-thymidylyl-(3′->5′)-2′-deoxy-P-thio-cytidylyl-(3′->5′)-P-thio-thymidylyl-(3′->5′)-2′-deoxy-P-thio-cytidylyl-(3′->5′)-2′-deoxy-P-thio-cytidylyl-(3′->5′)-P-thio-thymidylyl-(3′->5′)-P-thio-thymidylyl-(3′->5′)-2′-deoxy-P-thio-guanylyl-(3′->5′)-2′-deoxy-P-thio-guanylyl-(3′->5′)-2′-deoxy-P-thio-cytidylyl-(3′->5′)-2′-deoxy-guanosine sodium salt (RX-0201);
(j) VQD-002 [CAS No. 35943-35-2];
(k) LY294002 [CAS No. 154447-36-6];
(l) A-443654 [CAS No. 552325-16-3];
(m) A-674563 [CAS No. 552325-73-2];
(n) Akti-1;
(o) Akti-2;
(p) AR-42 [CAS No. 935881-37-1];
(q) API-59CJ-OMe [CAS No. 98510-80-6];
(r) ATI-13148;
(s) AZD-5363 [CAS No. 1143532-39-1];
(t) Erucylphosphocholine [CAS No. 143317-74-2];
(u) GSK-2141795 (GSK795) [CAS No. 1047634-65-0];
(v) KP372-1 [CAS No. 1374996-60-7];
(w) L-418;
(x) L-71-101;
(y) PBI-05204 [CAS No. 465-16-7]
(z) PIA5;
(aa) PX-316 [CAS No. 253440-95-8];
(ab) SR13668 [CAS No. 637774-61-9];
(ac) GSK 690693 (CAS #937174-76-0);
(ad) FPA 124 (CAS #902779-59-3);
(ae) Miltefosine [CAS No. 58066-85-6];
(af) PHT-427 (CAS #1 191951-57-1);
(ag) 10-DEBC hydrochloride [CAS No. 925681-41-0],
(ah) Akt inhibitor III ([(2R)-2-methoxy-3-octadecoxypropyl] (2,3,4-trihydroxycyclohexyl) hydrogen phosphate);
(ai) Akt inhibitor VIII [CAS 612847-09-3];
(aj) MK-2206 dihydrochloride (CAS #1032350-13-2);
(ak) SC79 [CAS No. 305834-79-1];
(al) AT7867 (CAS #857531-00-1);
(am) CCT128930 (CAS #885499-61-6);
(an) A-674563 (CAS #552325-73-2);
(ao) AGL 2263 [CAS No. 638213-98-6];
(ap) AS-041164 (5-benzo[1,3]dioxol-5-ylmethylene-thiazolidine-2,4-dione) [CAS No. 1146702-72-8];
(aq) BML-257 (CAS #32387-96-5);
(ar) XL-418 [CAS No. 871343-09-8];
(as) AKTI IX [CAS #98510-80-6];
(at) H-89 (CAS #127243-85-0);
(au) OXY-111A [CAS No. 802590-64-3]; (av) a salt of any of (a)-(au); and
(aw) any combination of (a)-(av).

The amount of the AKT inhibitor useful for the methods described herein can be an amount that is capable of reducing or inhibiting the activity of AKT in the one or more T cells (i.e., effective amount). The amount of the AKT inhibitor useful for the present disclosure can also be an amount that is capable of delaying or inhibiting maturation or differentiation of T cells in vitro. The one or more T cells can be contacted with an AKTi, for example, AKTi VIII, at a concentration of at least about 1 nM, at least about 10 nM, at least about 50 nM, at least about 100 nM, at least about 200 nM, at least about 300 nM, at least about 400 nM, at least about 500 nM, at least about 1 μM, at least about 2 μM, at least about 3 μM, at least about 4 μM, at least about 5 μM, at least about 6 μM, at least about 7 μM, at least about 8 μM, at least about 9 μM, at least about 10 μM, at least about 11 μM, at least about 12 μM, at least about 13 μM, at least about 14 μM, at least about 15 μM, at least about 16 μM, at least about 17 μM, at least about 18 μM, at least about 19 μM, at least about 20 μM, at least about 25 μM, at least about 30 μM, at least about 35 μM, at least about 40 μM, at least about 45 μM, at least about 50 μM, at least about 60 μM, at least about 70 μM, at least about 80 μM, at least about 90 μM, at least about 100 μM, at least about 200 μM, at least about 300 μM, at least about 400 μM, at least about 500 μtM, or at least about 1 mM.

The one or more T cells can be contacted with an AKT inhibitor, e.g., AKTi VIII, at a concentration of from about 1 nM to about 1 mM, from about 10 nM to about 1 mM, from about 100 nM to about 1 mM, from about 100 nM to about 500 μM, from about 100 nM to about 100 μM, from about 100 nM to about 50 μM, from about 100 nM to about 10 μM, from about 100 nM to about 1 μM, from about 100 nM to about 900 nM, from about 100 nM to about 800 nM, from about 100 nM to about 700 nM, from about 100 nM to about 600 nM, from about 100 nM to about 500 nM, from about 100 nM to about 400 nM, from about 100 nM to about 300 nM, from about 150 nM to about 300 nM, from about 200 nM to about 300 nM, from about 250 nM to about 300 nM, from about 1 μM to about 1 mM, from about 10 μM to about 1 mM, from about 100 μM to about 1 mM, from about 1 nM to about 100 μM, from about 1 nM to about 10 μM, from about 1 nM to about 1 μM, from about 1 nM to about 100 nM, from about 1 nM to about 50 nM, from about 100 nM to about 100 μM, from about 500 nM to about 50 μM, from about 1 μM to about 50 μM, from about 1 μM to about 10 μM, or from about 5 μM to about 10 μM.

### Tyrosine Kinase Inhibitor (TKi)

A “kinase inhibitor” as referred to herein is a molecular compound that inhibits one or more kinase(s) by binding to said kinase(s) and exerting an inhibiting effect on said kinase. A kinase inhibitor may be capable of binding to one or more kinase species, upon which the kinase activity of the one or more kinase is reduced. A kinase inhibitor as referred to herein is typically a small molecule, wherein a small molecule is a molecular compound of low molecular weight (typically less than 1 kDa) and size (a diameter which is typically smaller than 1 nm). In embodiments, the present methods concern the treatment of effector T cells with a kinase inhibitor for improving T cell efficacy, persistence, memory function, and/or antigen stimulated survival. In embodiments, the present methods concern the treatment of effector T cells with a kinase inhibitor for the improved production of Central Memory T (Tcm) cells. The kinase inhibitor may be a tyrosine kinase inhibitor (TKi). The kinase inhibitor may be a Src kinase inhibitor. The kinase inhibitor may be an Lck inhibitor. The kinase inhibitor may be dasatinib.

The tyrosine kinase inhibitor may be a Src kinase inhibitor. The tyrosine kinase inhibitor may be dasatinib, saracatinib, bosutinib, nilotinib, or PP1-inhibitor. The inhibitor may be bosutinib. The inhibitor may be saracatinib. The inhibitor may be nilotinib. The inhibitor may be PP1-inhibitor. The inhibitor may be dasatinib

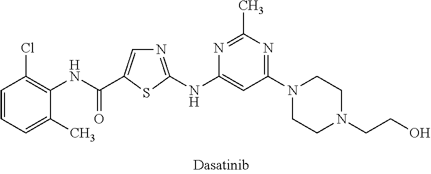 [SMILES: Cc1nc(Nc2ncc(C(=O)Nc3c(C)cccc3Cl)s2)cc(N2CCN(CCO)CC2)n1]

The structure of which is shown above, may correspond to CAS No. 302962-49-8. The AKTi used herein may be found in U.S. Pat. No. 6,596,746, the contents of which is hereby incorporated by reference in its entirety. An AKTi can be a crystalline monohydrate form of dasatinib (e.g., dasatinib monohydrate). Dasatinib monohydrate may correspond to CAS No. 863127-77-9.

It is to be understood that terms such as “a tyrosine kinase inhibitor” refer to the presence of a kinase inhibitor but do not exclude the possibility that additional kinase inhibitors, e.g. one, two, three or more additional kinase inhibitors could be present. In some embodiments in accordance with the invention, only one kinase inhibitor is used.

The one or more T cells can be contacted with an TKi, e.g., dasatinib, at a concentration of from about 1 nM to about 1 μM, from about 1 nM to about 500 nM, from about 1 nM to about 400 nM, from about 1 nM to about 300 nM, from about 1 nM to about 200 nM, from about 1 nM to about 150 nM, from about 1 nM to about 100 nM, from about 1 nM to about 50 nM, from about 1 nM to about 40 nM, from about 1 nM to about 30 nM, from about 1 nM to about 20 nM, from about 1 nM to about 10 nM, from about 2 nM to about 10 nM, from about 3 nM to about 10 nM, from about 4 nM to about 10 nM, from about 5 nM to about 10 nM, from about 100 nM to about 1 mM, from about 100 nM to about 500 μM, from about 100 nM to about 100 μM, from about 100 nM to about 50 μM, from about 100 nM to about 10 μM, from about 100 nM to about 1 μM, from about 100 nM to about 900 nM, from about 100 nM to about 800 nM, from about 100 nM to about 700 nM, from about 100 nM to about 600 nM, from about 100 nM to about 500 nM, from about 100 nM to about 400 nM, from about 100 nM to about 300 nM, from about 150 nM to about 300 nM, from about 200 nM to about 300 nM, from about 250 nM to about 300 nM, from about 1 μM to about 1 mM, from about 10 μM to about 1 mM, from about 100 μM to about 1 mM, from about 1 nM to about 100 μM, from about 1 nM to about 10 μM, from about 1 nM to about 1 μM, from about 1 nM to about 100 nM, from about 1 nM to about 50 nM, from about 100 nM to about 100 μM, from about 500 nM to about 50 μM, from about 1 μM to about 50 μM, from about 1 μM to about 10 μM, or from about 5 μM to about 10 μM.

### T-Cell Receptors

“T-cell receptor” (TCR) as used herein refers broadly to a protein receptor on T cells that is composed of a heterodimer of an alpha (a) and beta (β) chain, although in some cells the TCR consists of gamma and delta (γ/δ) chains. The TCR may be modified on any cell comprising a TCR, including a helper T cell, a cytotoxic T cell, a memory T cell, regulatory T cell, natural killer T cell, or a gamma delta T cell.

The TCR is generally found on the surface of T lymphocytes (or T cells) and is generally responsible for recognizing antigens bound to major histocompatibility complex (MHC) molecules. It is a heterodimer consisting of an alpha chain and a beta chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. In immunology, the CD3 antigen (CD stands for cluster of differentiation) is a protein complex composed of four distinct chains (CD3-γ chain, CD36 chain, and two CD3ε chains) in mammals, that associate with molecules T-cell receptor (TCR) and the ζ-chain to generate an activation signal in T lymphocytes. The TCR, ζ-chain, and CD3 molecules together comprise the TCR complex. The CD3-γ, CD3δ, and CD3ε chains are highly related cell surface proteins of the immunoglobulin superfamily containing a single extracellular immunoglobulin domain. The transmembrane region of the CD3 chains is negatively charged, a characteristic that allows these chains to associate with the positively charged TCR chains (TCRα and TCRβ). The intracellular tails of the CD3 molecules contain a single conserved motif known as an immunoreceptor tyrosine-based activation motif or ITAM for short, which is essential for the signaling capacity of the TCR.

A T-cell may co-express a T-cell receptor (TCR), antigen binding protein, or both, with modified CD8 polypeptides described herein, including, but are not limited to, those listed in Table 3 (SEQ ID NOs: 15-92). Further, a T-cell may express TCRs and antigen binding proteins described in U.S. Patent Application Publication No. 2017/0267738; U.S. Patent Application Publication No. 2017/0312350; U.S. Patent Application Publication No. 2018/0051080; U.S. Patent Application Publication No. 2018/0164315; U.S. Patent Application Publication No. 2018/0161396; U.S. Patent Application Publication No. 2018/0162922; U.S. Patent Application Publication No. 2018/0273602; U.S. Patent Application Publication No. 2019/0016801; U.S. Patent Application Publication No. 2019/0002556; U.S. Patent Application Publication No. 2019/0135914; U.S. Pat. Nos. 10,538,573; 10,626,160; U.S. Patent Application Publication No. 2019/0321478; U.S. Patent Application Publication No. 2019/0256572; U.S. Pat. Nos. 10,550,182; 10,526,407; U.S. Patent Application Publication No. 2019/0284276; U.S. Patent Application Publication No. 2019/0016802; U.S. Patent Application Publication No. 2019/0016803; U.S. Patent Application Publication No. 2019/0016804; U.S. Pat. No. 10,583,573; U.S. Patent Application Publication No. 2020/0339652; U.S. Pat. Nos. 10,537,624; 10,596,242; U.S. Patent Application Publication No. 2020/0188497; U.S. Pat. No. 10,800,845; U.S. Patent Application Publication No. 2020/0385468; U.S. Pat. Nos. 10,527,623; 10,725,044; U.S. Patent Application Publication No. 2020/0249233; U.S. Pat. No. 10,702,609; U.S. Patent Application Publication No. 2020/0254106; U.S. Pat. No. 10,800,832; U.S. Patent Application Publication No. 2020/0123221; U.S. Pat. Nos. 10,590,194; 10,723,796; U.S. Patent Application Publication No. 2020/0140540; U.S. Pat. No. 10,618,956; U.S. Patent Application Publication No. 2020/0207849; U.S. Patent Application Publication No. 2020/0088726; and U.S. Patent Application Publication No. 2020/0384028; the contents of each of these publications and sequence listings described therein are herein incorporated by reference in their entireties. The cell may be a αβ T cell, γδ T cell, natural killer T cell, natural killer cell, or combinations thereof. TCRs described herein may be single-chain TCRs or soluble TCRs.

Further, the TCRs that may be co-expressed with the modified CD8 polypeptides described herein in a T-cell may be TCRs comprised of an alpha chain (TCRα) and a beta chain (TCRβ). The TCRα chains and TCRβ chains that may be used in TCRs may be selected from R11KEA (SEQ ID NO: 15 and 16), R20P1H7 (SEQ ID NO: 17 and 18), R7P1D5 (SEQ ID NO: 19 and 20), R10P2G12 (SEQ ID NO: 21 and 22), R10P1A7 (SEQ ID NO: 23 and 24), R4P1D10 (SEQ ID NO: 25 and 26), R4P3F9 (SEQ ID NO: 27 and 28), R4P3H3 (SEQ ID NO: 29 and 30), R36P3F9 (SEQ ID NO: 31 and 32), R52P2G11 (SEQ ID NO: 33 and 34), R53P2A9 (SEQ ID NO: 35 and 36), R26P1A9 (SEQ ID NO: 37 and 38), R26P2A6 (SEQ ID NO: 39 and 40), R26P3H1 (SEQ ID NO: 41 and 42), R35P3A4 (SEQ ID NO: 43 and 44), R37P1C9 (SEQ ID NO: 45 and 46), R37P1H1 (SEQ ID NO: 47 and 48), R42P3A9 (SEQ ID NO: 49 and 50), R43P3F2 (SEQ ID NO: 51 and 52), R43P3G5 (SEQ ID NO: 53 and 54), R59P2E7 (SEQ ID NO: 55 and 56), R11P3D3 (SEQ ID NO: 57 and 58), R16P1C10 (SEQ ID NO: 59 and 60), R16P1E8 (SEQ ID NO: 61 and 62), R17P1A9 (SEQ ID NO: 63 and 64), R17P1D7 (SEQ ID NO: 65 and 66), R17P1G3 (SEQ ID NO: 67 and 68), R17P2B6 (SEQ ID NO: 69 and 70), R11P3D3KE (SEQ ID NO: 71 and 72), R39P1C12 (SEQ ID NO: 73 and 74), R39P1F5 (SEQ ID NO: 75 and 76), R40P1C2 (SEQ ID NO: 77 and 78), R41P3E6 (SEQ ID NO: 79 and 80), R43P3G4 (SEQ ID NO: 81 and 82), R44P3B3 (SEQ ID NO: 83 and 84), R44P3E7 (SEQ ID NO: 85 and 86), R49P2B7 (SEQ ID NO: 87 and 88), R55P1G7 (SEQ ID NO: 89 and 90), or R59P2A7 (SEQ ID NO: 91 and 92). The T-cell may be a αβ T cell, γδ T cell, or a natural killer T cell.

Table 1 shows examples of the peptides to which TCRs bind when the peptide is in a complex with an MHC molecule. (MHC molecules in humans may be referred to as HLA, human leukocyte-antigens).

TABLE 1
T-Cell Receptor and Peptides

| TCR name | Peptide (SEQ ID NO:) |
|---|---|
| R20P1H7, R7P1D5, R10P2G12 | KVLEHVVRV (SEQ ID NO: 215) |
| R10P1A7 | KIQEILTQV (SEQ ID NO: 123) |
| R4P1D10, R4P3F9, R4P3H3 | FLLDGSANV (SEQ ID NO: 238) |
| R36P3F9, R52P2G11, R53P2A9 | ILQDGQFLV (SEQ ID NO: 193) |
| R26P1A9, R26P2A6, R26P3H1, R35P3A4, | KVLEYVIKV (SEQ ID NO: 202) |
| R37P1C9, R37P1H1, R42P3A9, R43P3F2, | |
| R43P3G5, R59P2E7 | |
| R11KEA, R11P3D3, R16P1C10, R16P1E8, | SLLQHLIGL (SEQ ID NO: 147) |
| R17P1A9, R17P1D7, R17P1G3, R17P2B6, | |
| R11P3D3KE | |
| R39P1C12, R39P1F5, R40P1C2, R41P3E6, | ALSVLRLAL (SEQ ID NO: 248) |
| R43P3G4, R44P3B3, R44P3E7, R49P2B7, | |
| R55P1G7, R59P2A7 | |

### Tumor Associated Antigens (TAA)

Tumor associated antigen (TAA) peptides may be used with the CD8 polypeptides constructs, methods, uses, treatments and aspects described herein. For example, the T-cell receptors (TCRs) described herein may specifically bind to the TAA peptide when bound to a human leukocyte antigen (HLA). This is also known as a major histocompatibility complex (MHC) molecule. The MHC-molecules of the human are also designated as human leukocyte-antigens (HLA).

Tumor associated antigen (TAA) peptides that may be used with the CD8 polypeptides described herein include, but are not limited to, those listed in Table 3 and those TAA peptides described in U.S. Patent Application Publication No. 2016/0187351; U.S. Patent Application Publication No. 2017/0165335; U.S. Patent Application Publication No. 2017/0035807; U.S. Patent Application Publication No. 2016/0280759; U.S. Patent Application Publication No. 2016/0287687; U.S. Patent Application Publication No. 2016/0346371; U.S. Patent Application Publication No. 2016/0368965; U.S. Patent Application Publication No. 2017/0022251; U.S. Patent Application Publication No. 2017/0002055; U.S. Patent Application Publication No. 2017/0029486; U.S. Patent Application Publication No. 2017/0037089; U.S. Patent Application Publication No. 2017/0136108; U.S. Patent Application Publication No. 2017/0101473; U.S. Patent Application Publication No. 2017/0096461; U.S. Patent Application Publication No. 2017/0165337; U.S. Patent Application Publication No. 2017/0189505; U.S. Patent Application Publication No. 2017/0173132; U.S. Patent Application Publication No. 2017/0296640; U.S. Patent Application Publication No. 2017/0253633; U.S. Patent Application Publication No. 2017/0260249; U.S. Patent Application Publication No. 2018/0051080; U.S. Patent Application Publication No. 2018/0164315; U.S. Patent Application Publication No. 2018/0291082; U.S. Patent Application Publication No. 2018/0291083; U.S. Patent Application Publication No. 2019/0255110; U.S. Pat. Nos. 9,717,774; 9,895,415; U.S. Patent Application Publication No. 2019/0247433; U.S. Patent Application Publication No. 2019/0292520; U.S. Patent Application Publication No. 2020/0085930; U.S. Pat. Nos. 10,336,809; 10,131,703; 10,081,664; 10,081,664; 10,093,715; 10,583,573; and U.S. Patent Application Publication No. 2020/00085930; the contents of each of these publications, sequences, and sequence listings described therein are herein incorporated by reference in their entireties. The Tumor Associated Antigen (TAA) peptides described herein may be bound to an HLA (MHC) molecule. The Tumor Associated Antigen (TAA) peptides bound to an HLA may be recognized by a TCR described herein, optionally co-expressed with CD8 polypeptides described herein.

T cells may be engineered to express a chimeric antigen receptor (CAR) comprising a ligand binding domain derived from NKG2D, NKG2A, NKG2C, NKG2F, LLT1, AICL, CD26, NKRP1, NKp30, NKp44, NKp46, CD244 (2B4), DNAM-1, and NKp80, or an anti-tumor antibody such as anti-Her2neu or anti-EGFR and a signaling domain obtained from CD3-t, Dap 10, CD28, 4-IBB, and CD40L. In some examples, the chimeric receptor binds MICA, MICB, Her2neu, EGFR, mesothelin, CD38, CD20, CD 19, PSA, RON, CD30, CD22, CD37, CD38, CD56, CD33, CD30, CD138, CD123, CD79b, CD70, CD75, CA6, GD2, alpha-fetoprotein (AFP), carcinoembryonic antigen (CEA), CEACAM5, CA-125, MUC-16, 5T4, NaPi2b, ROR1, ROR2, 5T4, PLIF, Her2/Neu, EGFRvIII, GPMNB, LIV-1, glycolipidF77, fibroblast activating protein, PSMA, STEAP-1, STEAP-2, c-met, CSPG4, Nectin-4, VEGFR2, PSCA, folate binding protein/receptor, SLC44A4, Cripto, CTAG1B, AXL, IL-13R, IL-3R, SLTRK6, gp100, MART1, Tyrosinase, SSX2, SSX4, NYESO-1, epithelial tumor antigen (ETA), MAGEA family genes (such as MAGE3A. MAGE4A), KKLC1, mutated ras, βraf, p53, MHC class I chain-related molecule A (MICA), or MHC class I chain-related molecule B (MICB), HPV, or CMV. The T-cell may be a αβ T cell, γδ T cell, or a natural killer T cell.

Culturing T-Cells

Methods for the activation, transduction, and/or expansion of T cells, e.g., tumor-infiltrating lymphocytes, CD8+ T cells, CD4+ T cells, and T cells, that may be used for transgene expression are described herein. T cells may be activated, transduced, and expanded, while depleting α- and/or β-TCR positive cells. The T-cell may be a αβ T cell, γδ T cell, or a natural killer T cell.

Methods for the ex vivo expansion of a population of engineered γδ T-cells for adoptive transfer therapy are described herein. Engineered γδ T cells of the disclosure may be expanded ex vivo. Engineered T cells described herein can be expanded in vitro without activation by APCs, or without co-culture with APCs, and aminophosphates. Methods for transducing T cells are described in U.S. Patent Application No. 2019/0175650, published on Jun. 13, 2019, the contents of which are incorporated by reference in their entirety. Other methods for transduction and culturing of T-cells may be used.

T cells, including γδ T cells, may be isolated from a complex sample that is cultured in vitro. Whole PBMC population, without prior depletion of specific cell populations, such as monocytes, αβ T-cells, B-cells, and NK cells, can be activated and expanded. Enriched T cell populations can be generated prior to their specific activation and expansion. Activation and expansion of γδ T cells may be performed with or without the presence of native or engineered antigen presenting cells (APCs). Isolation and expansion of T cells from tumor specimens can be performed using immobilized T cell mitogens, including antibodies specific to γδ TCR, and other γδ TCR activating agents, including lectins. Isolation and expansion of γδ T cells from tumor specimens can be performed in the absence of γδ T cell mitogens, including antibodies specific to γδ TCR, and other γδ TCR activating agents, including lectins.

T cells, including γδ T cells, may be isolated from leukapheresis of a subject, for example, a human subject. In some embodiments, γδ T cells are not isolated from peripheral blood mononuclear cells (PBMC). The T cells may be isolated using anti-CD3 and anti-CD28 antibodies, optionally with recombinant human Interleukin-2 (rhIL-2), e.g., between about 50 and 150 U/mL rhIL-2.

The isolated T cells can rapidly expand in response to contact with one or more antigens. Some γδ T cells, such as Vγ9V62+ T cells, can rapidly expand in vitro in response to contact with some antigens, like prenyl-pyrophosphates, alkyl amines, and metabolites or microbial extracts during tissue culture. Stimulated T-cells can exhibit numerous antigen-presentation, co-stimulation, and adhesion molecules that can facilitate the isolation of T-cells from a complex sample. T cells within a complex sample can be stimulated in vitro with at least one antigen for 1 day, 2 days, 3 days, 4 days, 5 days, 6 days, 7 days, or another suitable period of time. Stimulation of T cells with a suitable antigen can expand a T cell population in vitro.

Activation and expansion of γδ T cells can be performed using activation and co-stimulatory agents described herein to trigger specific γδ T cell proliferation and persistence populations. Activation and expansion of γδ T-cells from different cultures can achieve distinct clonal or mixed polyclonal population subsets. Different agonist agents can be used to identify agents that provide specific γδ activating signals. Agents that provide specific γδ activating signals can be different monoclonal antibodies (MAbs) directed against the γδ TCRs. Companion co-stimulatory agents to assist in triggering specific γδ T cell proliferation without induction of cell energy and apoptosis can be used. These co-stimulatory agents can include ligands binding to receptors expressed on γδ cells, such as NKG2D, CD161, CD70, JAML, DNAX accessory molecule-1 (DNAM-1), ICOS, CD27, CD137, CD30, HVEM, SLAM, CD122, DAP, and CD28. Co-stimulatory agents can be antibodies specific to unique epitopes on CD2 and CD3 molecules. CD2 and CD3 can have different conformation structures when expressed on γδ or γδ T-cells. Specific antibodies to CD3 and CD2 can lead to distinct activation of γδ T cells.

Non-limiting examples of antigens that may be used to stimulate the expansion of T cells, including γδ T cells, from a complex sample in vitro may comprise, prenyl-pyrophosphates, such as isopentenyl pyrophosphate (IPP), alkyl-amines, metabolites of human microbial pathogens, metabolites of commensal bacteria, methyl-3-butenyl-1-pyrophosphate (2M3B1PP), (E)-4-hydroxy-3-methyl-but-2-enyl pyrophosphate (HMB-PP), ethyl pyrophosphate (EPP), farnesyl pyrophosphate (FPP), dimethylallyl phosphate (DMAP), dimethylallyl pyrophosphate (DMAPP), ethyl-adenosine triphosphate (EPPPA), geranyl pyrophosphate (GPP), geranylgeranyl pyrophosphate (GGPP), isopentenyl-adenosine triphosphate (IPPPA), monoethyl phosphate (MEP), monoethyl pyrophosphate (MEPP), 3-formyl-1-butyl-pyrophosphate (TUBAg 1), X-pyrophosphate (TUBAg 2), 3-formyl-1-butyl-uridine triphosphate (TUBAg 3), 3-formyl-1-butyl-deoxythymidine triphosphate (TUBAg 4), monoethyl alkylamines, allyl pyrophosphate, crotoyl pyrophosphate, dimethylallyl-γ-uridine triphosphate, crotoyl-γ-uridine triphosphate, allyl-γ-uridine triphosphate, ethylamine, isobutylamine, sec-butylamine, iso-amylamine and nitrogen containing bisphosphonates.

A population of T-cells, including γδ T cells, may be expanded ex vivo prior to engineering of the T-cells. Non-limiting example of reagents that can be used to facilitate the expansion of a T-cell population in vitro may comprise anti-CD3 or anti-CD2, anti-CD27, anti-CD30, anti-CD70, anti-OX40 antibodies, IL-2, IL-15, IL-12, IL-9, IL-33, IL-18, or IL-21, CD70 (CD27 ligand), phytohaemagglutinin (PHA), concavalin A (ConA), pokeweed (PWM), protein peanut agglutinin (PNA), soybean agglutinin (SBA), Les Culinaris Agglutinin (LCA), Pisum Sativum Agglutinin (PSA), Helix pomatia agglutinin (HPA), Vicia graminea Lectin (VGA), or another suitable mitogen capable of stimulating T-cell proliferation. Further, the T-cells may be expanded using MCSF, IL-6, eotaxin, IFN-alpha, IL-7, gamma-induced protein 10, IFN-gamma, IL-1RA, IL-12, MIP-1alpha, IL-2, IL-13, MIP-1beta, IL-2R, IL-15, and combinations thereof.

The ability of γδ T cells to recognize a broad spectrum of antigens can be enhanced by genetic engineering of the γδ T cells. The γδ T cells can be engineered to provide a universal allogeneic therapy that recognizes an antigen of choice in vivo. Genetic engineering of the γδ T-cells may comprise stably integrating a construct expressing a tumor recognition moiety, such as αβ TCR, γδ TCR, chimeric antigen receptor (CAR), which combines both antigen-binding and T-cell activating functions into a single receptor, an antigen binding fragment thereof, or a lymphocyte activation domain into the genome of the isolated γδ T-cell(s), a cytokine (for example, IL-15, IL-12, IL-2. IL-7. IL-21, IL-18, IL-19, IL-33, IL-4, IL-9, IL-23, or IL1β) to enhance T-cell proliferation, survival, and function ex vivo and in vivo. Genetic engineering of the isolated γδ T-cell may also include deleting or disrupting gene expression from one or more endogenous genes in the genome of the isolated γδ T-cells, such as the MHC locus (loci).

Engineered (or transduced) T cells, including γδ T cells, can be expanded ex vivo without stimulation by an antigen presenting cell or aminobisphosphonate. Antigen reactive engineered T cells of the present disclosure may be expanded ex vivo and in vivo. An active population of engineered T cells may be expanded ex vivo without antigen stimulation by an antigen presenting cell, an antigenic peptide, a non-peptide molecule, or a small molecule compound, such as an aminobisphosphonate but using certain antibodies, cytokines, mitogens, or fusion proteins, such as IL-17 Fc fusion, MICA Fc fusion, and CD70 Fc fusion. Examples of antibodies that can be used in the expansion of a γδ T-cell population include anti-CD3, anti-CD27, anti-CD30, anti-CD70, anti-OX40, anti-NKG2D, or anti-CD2 antibodies, examples of cytokines may comprise IL-2, IL-15, IL-12, IL-21, IL-18, IL-9, IL-7, and/or IL-33, and examples of mitogens may comprise CD70 the ligand for human CD27, phytohaemagglutinin (PHA), concavalin A (ConA), pokeweed mitogen (PWM), protein peanut agglutinin (PNA), soybean agglutinin (SBA), les culinaris agglutinin (LCA), Pisum sativum agglutinin (PSA), Helix pomatia agglutinin (HPA), Vicia graminea Lectin (VGA) or another suitable mitogen capable of stimulating T-cell proliferation.

A population of engineered T cells, including γδ T cells, can be expanded in less than 60 days, less than 48 days, less than 36 days, less than 24 days, less than 12 days, or less than 6 days. A population of engineered T cells can be expanded from about 7 days to about 49 days, about 7 days to about 42 days, from about 7 days to about 35 days, from about 7 days to about 28 days, from about 7 days to about 21 days, or from about 7 days to about 14 days. The T-cells may be expanded for between about 1 and 21 days. For example, the T-cells may be expanded for about at least 1, 2, 3, 4, 5, 6, 7, 8, 9, 10, 11, 12, 13, 14, 15, 16, 17, 18, 19, or 20 days.

The same methodology may be used to isolate, activate, and expand αβ T cells.

The same methodology may be used to isolate, activate, and expand γδ T cells.

In an aspect, activation described herein may be carried out within a period of no more than about 1 hour, no more than about 2, hours, no more than about 3 hours, no more than about 4 hours, no more than about 5 hours, no more than about 6 hours, no more than about 7 hours, no more than about 8 hours, no more than about 9 hours, no more than about 10 hours, no more than about 11 hours, no more than about 12 hours, no more than about 14 hours, no more than about 16 hours, no more than about 18 hours, no more than about 20 hours, no more than about 22 hours, no more than about 24 hours, no more than about 26 hours, no more than about 28 hours, no more than about 30 hours, no more than about 36 hours, no more than about 48 hours, no more than about 60 hours, no more than about 72 hours, no more than about 84 hours, no more than about 96 hours, no more than about 108 hours, or no more than about 120 hours.

In another aspect, activation described herein may be carried out within a period of from about 1 hour to about 120 hours, about 1 hour to about 108 hours, about 1 hour to about 96 hours, about 1 hour to about 84 hours, about 1 hour to about 72 hours, about 1 hour to about 60 hours, about 1 hour to about 48 hours, about 1 hour to about 36 hours, about 1 hour to about 24 hours, about 2 hours to about 24 hours, about 4 hours to about 24 hours, about 6 hours to about 24 hours, about 8 hours to about 24 hours, about 10 hours to about 24 hours, about 12 hours to about 24 hours, about 12 hours to about 72 hours, about 24 hours to about 72 hours, about 6 hours to about 48 hours, about 24 hours to about 48 hours, about 6 hours to about 72 hours, or about 1 hours to about 12 hours.

In an aspect, transduction described herein may be carried out within a period of no more than about 1 hour, no more than about 2 hours, no more than about 3 hours, no more than about 4 hours, no more than about 5 hours, no more than about 6 hours, no more than about 7 hours, no more than about 8 hours, no more than about 9 hours, no more than about 10 hours, no more than about 11 hours, no more than about 12 hours, no more than about 14 hours, no more than about 16 hours, no more than about 18 hours, no more than about 20 hours, no more than about 22 hours, no more than about 24 hours, no more than about 26 hours, no more than about 28 hours, no more than about 30 hours, no more than about 36 hours, no more than about 42 hours, no more than about 48 hours, no more than about 54 hours, no more than about 60 hours, no more than about 66 hours, no more than about 72 hours, no more than about 84 hours, no more than about 96 hours, no more than about 108 hours, or no more than about 120 hours.

In another aspect, transduction described herein may be carried out within a period of from about 1 hour to 120 hours, about 1 hour to 108 hours, about 1 hour to 96 hours, about 1 hour to 72 hours, about 1 hour to 48 hours, about 1 hour to 36 hours, about 1 hour to 24 hours, about 2 hour to 24 hours, about 4 hour to 24 hours, about 6 hour to 24 hours, about 8 hour to 24 hours, about 10 hour to 24 hours, about 12 hour to 24 hours, about 14 hour to 24 hours, about 16 hour to 24 hours, about 18 hour to 24 hours, about 20 hour to 24 hours, or about 22 hour to 24 hours.

In an aspect, expansion described herein may be carried out within a period of no more than about 1 day, no more than about 2 days, no more than about 3 days, no more than about 4 days, no more than about 5 days, no more than about 6 days, no more than about 7 days, no more than about 8 days, no more than about 9 days, no more than about 10 days, no more than about 15 days, no more than about 20 days, no more than about 25 days, or no more than about 30 days.

In another aspect, expansion described herein may be carried out within a period of from about 1 day to about 30 days, about 1 day to about 25 days, about 1 day to about 20 days, about 1 day to about 15 days, about 1 day to about 10 days, about 2 days to about 10 days, about 3 days to about 10 days, about 4 days to about 10 days, about 5 days to about 10 days, about 6 days to about 10 days, about 7 days to about 10 days, about 8 days to about 10 days, or about 9 days to about 10 days.

### Vectors

Engineered T-cells may be generated using various methods, including those recognized in the literature. For example, a polynucleotide encoding an expression cassette that comprises a tumor recognition, or another type of recognition moiety, can be stably introduced into the T-cell by a transposon/transposase system or a viral-based gene transfer system, such as a lentiviral or a retroviral system, or another suitable method, such as transfection, electroporation, transduction, lipofection, calcium phosphate (CaPO₄), nanoengineered substances, such as Ormosil, viral delivery methods, including adenoviruses, retroviruses, lentiviruses, adeno-associated viruses, or another suitable method. A number of viral methods have been used for human gene therapy, such as the methods described in WO 1993/020221, the content of which is incorporated herein in its entirety. Non-limiting examples of viral methods that can be used to engineer T cells may comprise y-retroviral, adenoviral, lentiviral, herpes simplex virus, vaccinia virus, pox virus, or adeno-virus associated viral methods. The T cells may be αβ T cells or γδ T cells.

Viruses used for transfection of T-cells include naturally occurring viruses as well as artificial viruses. Viruses may be either an enveloped or non-enveloped virus. Parvoviruses (such as AAVs) are examples of non-enveloped viruses. The viruses may be enveloped viruses. The viruses used for transfection of T-cells may be retroviruses and in particular lentiviruses. Viral envelope proteins that can promote viral infection of eukaryotic cells may comprise HIV-1 derived lentiviral vectors (LVs) pseudotyped with envelope glycoproteins (GPs) from the vesicular stomatitis virus (VSV-G), the modified feline endogenous retrovirus (RD114TR) (SEQ ID NO: 97), and the modified gibbon ape leukemia virus (GALVTR). These envelope proteins can efficiently promote entry of other viruses, such as parvoviruses, including adeno-associated viruses (AAV), thereby demonstrating their broad efficiency. For example, other viral envelop proteins may be used including Moloney murine leukemia virus (MLV) 4070 env (such as described in Merten et al., J. Virol. 79:834-840, 2005; the content of which is incorporated herein by reference), RD114 env, chimeric envelope protein RD114pro or RDpro (which is an RD114-HIV chimera that was constructed by replacing the R peptide cleavage sequence of RD114 with the HIV-1 matrix/capsid (MA/CA) cleavage sequence, such as described in Bell et al. Experimental Biology and Medicine 2010; 235: 1269-1276; the content of which is incorporated herein by reference), baculovirus GP64 env (such as described in Wang et al. J. Virol. 81:10869-10878, 2007; the content of which is incorporated herein by reference), or GALV env (such as described in Merten et al., J. Virol. 79:834-840, 2005; the content of which is incorporated herein by reference), or derivatives thereof.

A single lentiviral cassette can be used to create a single lentiviral vector, expressing at least four individual monomer proteins of two distinct dimers from a single multi-cistronic mRNA so as to co-express the dimers on the cell surface. For example, the integration of a single copy of the lentiviral vector was sufficient to transform T cells to co-express TCRαβ and CD8αβ, optionally αβ T cells or γδ T cells.

Vectors may comprise a multi-cistronic cassette within a single vector capable of expressing more than one, more than two, more than three, more than four genes, more than five genes, or more than six genes, in which the polypeptides encoded by these genes may interact with one another or may form dimers. The dimers may be homodimers, e.g., two identical proteins forming a dimer, or heterodimers, e.g., two structurally different proteins forming a dimer.

Additionally, multiple vectors may be used to transfect cells with the constructs and sequences described herein. For example, the TCR transgene may be on one vector and the CD8 transgene encoding a polypeptide described herein may be on a second that are transfected either simultaneously or sequentially using recognized methods. A T-cell line may be stably transfected with a CD8 transgene encoding a CD8 polypeptide described herein and then sequentially transfected with a TCR transgene or visa verse.

The transgene may further include one or more multicistronic element(s) and the multicistronic element(s) may be positioned, for example, between the nucleic acid sequence encoding the TCRα or a portion thereof and the nucleic acid sequence encoding the TCRβ or a portion thereof; between the nucleic acid sequence encoding the CD8a or a portion thereof and the nucleic acid sequence encoding the CD8β or a portion thereof, or between any two nucleic acid sequences encoding of TCRα, TCRβ, CD8α, and CD8β. In embodiments, the multicistronic element(s) may include a sequence encoding a ribosome skip element selected from among a T2A, a P2A, a E2A or a F2A or an internal ribosome entry site (IRES). As used herein, the term “self-cleaving 2A peptide” refers to relatively short peptides (of the order of 20 amino acids long, depending on the virus of origin) acting co-translationally, by preventing the formation of a normal peptide bond between the glycine and last proline, resulting in the ribosome skipping to the next codon, and the nascent peptide cleaving between the Gly and Pro. After cleavage, the short 2A peptide remains fused to the C-terminus of the ‘upstream’ protein, while the proline is added to the N-terminus of the ‘downstream’ protein. Self-cleaving 2A peptide may be selected from porcine teschovirus-1 (P2A), equine rhinitis A virus (E2A), Thosea asigna virus (T2A), foot-and-mouth disease virus (F2A), or any combination thereof (see, e.g., Kim et al., PLOS One 6:e18556, 2011, the content of which including 2A nucleic acid and amino acid sequences are incorporated herein by reference in their entireties). By adding the linker sequences (GSG or SGSG (SEQ ID NO: 266)) before the self-cleaving 2A sequence, this may enable efficient synthesis of biologically active proteins, e.g., TCRs.

As used herein, the term “internal ribosome entry site (IRES)” refers to a nucleotide sequence located in a messenger RNA (mRNA) sequence, which can initiate translation without relying on the 5′ cap structure. IRES is usually located in the 5′ untranslated region (5′UTR) but may also be located in other positions of the mRNA. IRES may be selected from IRES from viruses, IRES from cellular mRNAs, in particular IRES from picornavirus, such as polio, EMCV and FMDV, flavivirus, such as hepatitis C virus (HCV), pestivirus, such as classical swine fever virus (CSFV), retrovirus, such as murine leukaemia virus (MLV), lentivirus, such as simian immunodeficiency virus (SIV), and insect RNA virus, such as cricket paralysis virus (CRPV), and IRES from cellular mRNAs, e.g. translation initiation factors, such as eIF4G, and DAPS, transcription factors, such as c-Myc, and NF-κB-repressing factor (NRF), growth factors, such as vascular endothelial growth factor (VEGF), fibroblast growth factor 2 (FGF-2), platelet-derived growth factor B (PDGF-B), homeotic genes, such as antennapedia, survival proteins, such as X-linked inhibitor of apoptosis (XIAP), and Apaf-1, and other cellular mRNA, such as BiP.

Constructs and vectors described herein are used with the methodology described in U.S. Patent Application Publication No. 2019/0175650, published on Jun. 13, 2019, the contents of which are incorporated by reference in their entirety.

Non-viral vectors may also be used with the sequences, constructs, and cells described herein.

The cells may be transfected by other means known in the art including lipofection (liposome-based transfection), electroporation, calcium phosphate transfection, biolistic particle delivery (e.g., gene guns), microinjection, or combinations thereof. Various methods of transfecting cells are known in the art. See, e.g., Sambrook & Russell (Eds.) Molecular Cloning: A Laboratory Manual (3rd Ed.) Volumes 1-3 (2001) Cold Spring Harbor Laboratory Press; Ramamoorth & Narvekar “Non Viral Vectors in Gene Therapy—An Overview.” J Clin Diagn Res. (2015) 9(1): GE01-GE06.

### Compositions

Compositions may comprise the modified CD8 polypeptides described herein. Further, compositions described herein may comprise a T-cell expressing CD8 polypeptides described herein. The compositions described herein may comprise a T-cell expressing CD8 polypeptides described herein and a T-cell receptor (TCR), optionally a TCR that specifically binds one of the TAA described herein complexed with an antigen presenting protein, e.g., MHC, referred to as HLA in humans, for human leukocyte antigen.

To facilitate administration, the T cells described herein can be made into a pharmaceutical composition or made into an implant appropriate for administration in vivo, with pharmaceutically acceptable carriers or diluents. The means of making such a composition or an implant are described in the art. See, e.g., Remington's Pharmaceutical Sciences, 16th Ed., Mack, ed. (1980).

The T cells described herein can be formulated into a preparation in semisolid or liquid form, such as a capsule, solution, infusion, or injection. Means known in the art can be utilized to prevent or minimize release and absorption of the composition until it reaches the target tissue or organ, or to ensure timed-release of the composition. Desirably, however, a pharmaceutically acceptable form is employed that does not hinder the cells from expressing the CARs or TCRs. Thus, desirably the T cells described herein can be made into a pharmaceutical composition comprising a carrier. The T cells described herein can be formulated with a physiologically acceptable carrier or excipient to prepare a pharmaceutical composition. The carrier and composition can be sterile. Exemplary carriers include, for example, a balanced salt solution, such as Hanks' balanced salt solution, or normal saline. The formulation should suit the mode of administration. Suitable pharmaceutically acceptable carriers include but are not limited to water, salt solutions (e.g., NaCl), saline, buffered saline, as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, that do not deleteriously react with the T-cells. The T-cells may be αβ T cells or γδ T cells that express CD8 polypeptides described herein, optionally a TCR described herein.

A composition of the present invention can be provided in unit dosage form wherein each dosage unit, e.g., an injection, contains a predetermined amount of the composition, alone or in appropriate combination with other active agents.

The compositions described herein may be a pharmaceutical composition. Pharmaceutical composition described herein may further comprise an adjuvant selected from the group consisting of colony-stimulating factors, including but not limited to Granulocyte Macrophage Colony Stimulating Factor (GM-CSF, sargramostim), cyclophosphamide, imiquimod, resiquimod, interferon-alpha, or a combination thereof.

Pharmaceutical composition described herein may comprise an adjuvant selected from the group consisting of colony-stimulating factors, e.g., Granulocyte Macrophage Colony Stimulating Factor (GM-CSF, sargramostim), cyclophosphamide, imiquimod and resiquimod.

Exemplary adjuvants include but are not limited to cyclophosphamide, imiquimod or resiquimod. Other exemplary adjuvants include Montanide IMS 1312, Montanide ISA 206, Montanide ISA 50V, Montanide ISA-51, poly-ICLC (Hiltonol®) and anti-CD40 mAB, or combinations thereof.

Other examples for useful adjuvants include, but are not limited to chemically modified CpGs (e.g. CpR, Idera), dsRNA analogues such as Poly(I:C) and derivates thereof (e.g. AmpliGen®, Hiltonol®, poly-(ICLC), poly(IC-R), poly(LC12U), non-CpG bacterial DNA or RNA as well as immunoactive small molecules and antibodies such as cyclophosphamide, sunitinib, immune checkpoint inhibitors including ipilimumab, nivolumab, pembrolizumab, atezolizumab, avelumab, durvalumab, and cemiplimab, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present invention can readily be determined by the skilled artisan without undue experimentation.

Other adjuvants include but are not limited to anti-CD40, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, and particulate formulations with poly(lactide co-glycolide) (PLG), Polyinosinic-polycytidylic acid-poly-1-lysine carboxymethylcellulose (poly-ICLC), virosomes, and/or interleukin-1 (IL-1), IL-2, IL-4, IL-7, IL-12, IL-13, IL-15, IL-18, IL-21, and IL-23. See, e.g., Narayanan et al. J. Med. Chem. (2003) 46(23): 5031-5044; Pohar et al. Scientific Reports 7 14598 (2017); Grajkowski et al. Nucleic Acids Research (2005) 33(11): 3550-3560; Martins et al. Expert Rev Vaccines (2015) 14(3): 447-59.

The composition described herein may also include one or more adjuvants. Adjuvants are substances that non-specifically enhance or potentiate the immune response (e g, immune responses mediated by CD8-positive T cells and helper-T (TH) cells to an antigen and would thus be considered useful in the medicament of the present invention. Suitable adjuvants include, but are not limited to, 1018 ISS, aluminium salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, JuvImmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactide co-glycolide) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. The adjuvants may be Freund's or GM-CSF. Several immunological adjuvants (e.g., MF59) specific for dendritic cells and their preparation have been described previously. Also, cytokines may be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (U.S. Pat. No. 5,849,589, incorporated herein by reference in its entirety) and acting as immunoadjuvants (e.g., IL-12, IL-15, IL-23, IL-7, IFN-alpha. IFN-beta).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, 2006). U.S. Pat. No. 6,406,705 B1 describes the combined use of CpG oligonucleotides, non-nucleic acid adjuvants and an antigen to induce an antigen-specific immune response. An exemplary CpG TLR9 antagonist is dSLIM (double Stem Loop Immunomodulator) by Mologen (Berlin, Germany) dSLIM may be a component of the pharmaceutical composition described herein. Other TLR binding molecules such as RNA binding TLR 7, TLR 8 and/or TLR 9 may also be used.

### Methods of Treatment and Preparing

Engineered T cells may express modified CD8 polypeptides described herein. Further, the Engineered T cells may express a TCR described herein. The TCR expressed by the engineered T cells may recognize a TAA bound to an HLA as described herein. Engineered T cells of the present disclosure can be used to treat a subject in need of treatment for a condition, for example, a cancer described herein. The T cells may be αβ T cells or γδ T cells that express a modified CD8 polypeptide, optionally a TCR described herein.

A method of treating a condition (e.g., ailment) in a subject with T cells described herein may comprise administering to the subject a therapeutically effective amount of engineered T cells described herein, optionally γδ T cells. T cells described herein may be administered at various regimens (e.g., timing, concentration, dosage, spacing between treatment, and/or formulation). A subject can also be preconditioned with, for example, chemotherapy, radiation, or a combination of both, prior to receiving engineered T cells of the present disclosure. A population of engineered T cells may also be frozen or cryopreserved prior to being administered to a subject. A population of engineered T cells can include two or more cells that express identical, different, or a combination of identical and different tumor recognition moieties. For instance, a population of engineered T-cells can include several distinct engineered T cells that are designed to recognize different antigens, or different epitopes of the same antigen. The T cells may be αβ T cells or γδ T cells that express a CD8 polypeptide described herein, optionally a TCR described herein.

T cells described herein, including αβ T-cells and γδ T cells, may be used to treat various conditions. The T cells may be αβ T cells or γδ T cells that express a CD8 polypeptide, optionally a TCR described herein. T cells described herein may be used to treat a cancer, including solid tumors and hematologic malignancies. Non-limiting examples of cancers include: non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

The T cells described herein may be used to treat an infectious disease. The T cells described herein may be used to treat an infectious disease, an infectious disease may be caused a virus. The T cells described herein may be used to treat an immune disease, such as an autoimmune disease. The T cells may be αβ T cells or γδ T cells that express a CD8 polypeptide, optionally a TCR described herein.

Treatment with T cells described herein, optionally γδ T cells, may be provided to the subject before, during, and after the clinical onset of the condition. Treatment may be provided to the subject after 1 day, 1 week, 6 months, 12 months, or 2 years after clinical onset of the disease. Treatment may be provided to the subject for more than 1 day, 1 week, 1 month, 6 months, 12 months, 2 years, 3 years, 4 years, 5 years, 6 years, 7 years, 8 years, 9 years, 10 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can include administering to a subject a pharmaceutical composition comprising engineered T cells described herein. The T cells may be αβ T cells or γδ T cells that express a CD8 polypeptide, optionally a TCR described herein.

Administration of engineered T cells of the present disclosure to a subject may modulate the activity of endogenous lymphocytes in a subject's body. Administration of engineered T cells to a subject may provide an antigen to an endogenous T-cell and may boost an immune response. The memory T cell may be a CD4+ T-cell. The memory T cell may be a CD8+ T-cell. Administration of engineered T cells of the present disclosure to a subject may activate the cytotoxicity of another immune cell. The other immune cell may be a CD8+ T-cell. The other immune cell may be a Natural Killer T-cell. Administration of engineered γδ T-cells of the present disclosure to a subject may suppress a regulatory T-cell. The regulatory T-cell may be a FOX3+ Treg cell. The regulatory T-cell may be a FOX3− Treg cell. Non-limiting examples of cells whose activity can be modulated by engineered T cells of the disclosure may comprise: hematopioietic stem cells; B cells; CD4; CD8; red blood cells; white blood cells; dendritic cells, including dendritic antigen presenting cells; leukocytes; macrophages; memory B cells; memory T-cells; monocytes; natural killer cells; neutrophil granulocytes; T-helper cells; and T-killer cells. The T cells may be αβ T cells or γδ T cells that express a CD8 polypeptide, optionally a TCR described herein.

During most bone marrow transplants, a combination of cyclophosphamide with total body irradiation may be conventionally employed to prevent rejection of the hematopietic stem cells (HSC) in the transplant by the subject's immune system. Incubation of donor bone marrow with interleukin-2 (IL-2) ex vivo may be performed to enhance the generation of killer lymphocytes in the donor marrow. Interleukin-2 (IL-2) is a cytokine that may be necessary for the growth, proliferation, and differentiation of wild-type lymphocytes. Current studies of the adoptive transfer of γδ T-cells into humans may require the co-administration of γδ T-cells and interleukin-2. However, both low- and high-dosages of IL-2 can have highly toxic side effects. IL-2 toxicity can manifest in multiple organs/systems, most significantly the heart, lungs, kidneys, and central nervous system. The disclosure provides a method for administrating engineered T cells to a subject without the co-administration of a native cytokine or modified versions thereof, such as IL-2, IL-15, IL-12, IL-21. Engineered T cells can be administered to a subject without co-administration with IL-2. Engineered T cells may be administered to a subject during a procedure, such as a bone marrow transplant without the co-administration of IL-2.

The methods may further comprise administering a chemotherapy agent. The dosage of the chemotherapy agent may be sufficient to deplete the patient's T-cell population. The chemotherapy may be administered about 5-7 days prior to T-cell administration. The chemotherapy agent may be cyclophosphamide, fludarabine, or a combination thereof. The chemotherapy agent may comprise dosing at about 400-600 mg/m²/day of cyclophosphamide. The chemotherapy agent may comprise dosing at about 10-30 mg/m²/day of fludarabine.

The methods may further comprise pre-treatment of the patient with low-dose radiation prior to administration of the composition comprising T-cells. The low dose radiation may comprise about 1.4 Gy for 1-6 days, such as about 5 days, prior to administration of the composition comprising T-cells.

The patient may be HLA-A*02.

The patient may be HLA-A*06.

The methods may further comprise administering an anti-PD1 antibody. The anti-PD1 antibody may be a humanized antibody. The anti-PD1 antibody may be pembrolizumab. The dosage of the anti-PD1 antibody may be about 200 mg. The anti-PD1 antibody may be administered every 3 weeks following T-cell administration.

The dosage of T-cells may be between about 0.8-1.2×10⁹ T cells. The dosage of the T cells may be about 0.5×10⁸ to about 10×10⁹ T cells. The dosage of T-cells may be about 1.2-3×10⁹ T cells, about 3-6×10⁹ T cells, about 10×10⁹ T cells, about 5×10⁹ T cells, about 0.1×10⁹ T cells, about 1×10⁸ T cells, about 5×10⁸ T cells, about 1.2-6×10⁹ T cells, about 1-6×10⁹ T cells, or about 1-8×10⁹ T cells.

The T cells may be administered in 3 doses. The T-cell doses may escalate with each dose. The T-cells may be administered by intravenous infusion.

The CD8 sequences described herein and associated products and compositions may be used autologous or allogenic methods of adoptive cellular therapy CD8 sequences, T cells thereof, and compositions may be used in, for example, methods described in U.S. Patent Application Publication 2019/0175650; U.S. Patent Application Publication 2019/0216852; U.S. Patent Application Publication 2019/024743; and U.S. Provisional Patent Application 62/980,844, each of which are incorporated by reference in their entireties.

The disclosure also provides for a population of modified T cells that present an exogenous CD8 polypeptide described herein and a T cell receptor wherein the population of modified T cells is activated and expanded with a combination of IL-2 and IL-15. The population of modified T cells may be expanded and/or activated with a combination of IL-2, IL-15, and zoledronate. The population of modified T cells may be activated with a combination of IL-2, IL-15, and zoledronate while expanded with a combination of IL-2, IL-15, and without zoledronate. The disclosure further provides for use of other interleukins during activation and/or expansion, such as IL-12, IL-18, IL-21, and combinations thereof.

In some aspects, IL-21, a histone deacetylase inhibitor (HDACi), or combinations thereof may be utilized in the field of cancer treatment, with methods described herein, and/or with ACT processes described herein. The present disclosure provides methods for reprogramming effector T cells to a central memory phenotype comprising culturing the effector T cells with at least one HDACi together with IL-21. Representative HDACi include, for example, trichostatin A, trapoxin B, phenylbutyrate, valproic acid, vorinostat (suberanilohydroxamic acid), belinostat, panobinostat, dacinostat, entinostat, tacedinaline, and mocetinostat.

Compositions comprising engineered T cells described herein may be administered for prophylactic and/or therapeutic treatments. The compositions described herein may be pharmaceutical compositions. Pharmaceutical compositions may comprise a therapeutically effective amount of engineered T cells as described herein, and optionally a pharmaceutically acceptable excipient and/or adjuvant. In therapeutic applications, pharmaceutical compositions can be administered to a subject already suffering from a disease or condition in an amount sufficient to cure or at least partially arrest the symptoms of the disease or condition. An engineered T-cell can also be administered to lessen a likelihood of developing, contracting, or worsening a condition. Effective amounts of a population of engineered T-cells for therapeutic use can vary based on the severity and course of the disease or condition, previous therapy, the subject's health status, weight, and/or response to the drugs, and/or the judgment of the treating physician. The T cells may be αβ T cells or γδ T cells engineered to express modified CD8 polypeptides described herein and optionally a TCR described herein. T-cell therapy has been successful in treating various cancers. Li et al. Signal Transduction and Targeted Therapy 4(35): (2019), the content of which is incorporated by reference in its entirety.

### Methods of Administration

In the methods or uses described herein, one or multiple engineered T cell populations or pharmaceutical compositions described herein may be administered to a subject in any order or simultaneously. If simultaneously, the multiple engineered T cells or pharmaceutical compositions can be provided in a single, unified form, such as an intravenous injection, or in multiple forms, for example, as multiple intravenous infusions, subcutaneous injections or pills. Engineered T-cells or pharmaceutical compositions can be packed together or separately, in a single package or in a plurality of packages. One or all of the engineered T cells or pharmaceutical compositions can be given in multiple doses. If not simultaneous, the timing between the multiple doses may vary to as much as about a week, a month, two months, three months, four months, five months, six months, or about a year. Engineered T cells can expand within a subject's body, in vivo, after administration of said engineered T cells or pharmaceutical compositions to a subject. Engineered T cells or pharmaceutical compositions can be frozen to provide cells for multiple treatments with the same cell preparation. Engineered T cells of the present disclosure, and pharmaceutical compositions comprising the same, can be packaged as a kit. A kit may comprise instructions (e.g., written instructions) on the use of engineered T cells and compositions comprising the same.

A use or method of treating a cancer described herein may comprise administering to a subject a therapeutically-effective amount of engineered T cells or a pharmaceutical composition as described herein, in which the administration treats the cancer. The therapeutically-effective amount of engineered γδ T cells may be administered for at least about 10 seconds, 30 seconds, 1 minute, 10 minutes, 30 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 1 month, 2 months, 3 months, 4 months, 5 months, 6 months, or 1 year. The therapeutically-effective amount of the engineered T cells or pharmaceutical compositions may be administered for at least one week. The therapeutically-effective amount of engineered T cells may be administered for at least two weeks.

Engineered T-cells or pharmaceutical compositions described herein, optionally γδ T cells, can be administered before, during, or after the occurrence of a disease or condition, and the timing of administering a pharmaceutical composition comprising an engineered T-cell can vary. For example, engineered T cells or pharmaceutical compositions can be used as a prophylactic and can be administered continuously to subjects with a propensity to conditions or diseases in order to lessen the likelihood of occurrence of the disease or condition. Engineered T-cells or pharmaceutical compositions can be administered to a subject during or as soon as possible after the onset of the symptoms. The administration of engineered T cells or pharmaceutical compositions can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical, such as by any route described herein using any formulation described herein. The administration of engineered T cells or pharmaceutical compositions of the present disclosure may be an intravenous administration. One or multiple dosages of engineered T cells or pharmaceutical compositions can be administered as soon as is practicable after the onset of a cancer, an infectious disease, an immune disease, sepsis, or with a bone marrow transplant, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of engineered T cells or pharmaceutical compositions can be administered years after onset of the cancer and before or after other treatments. Engineered γδ T cells or pharmaceutical compositions described herein can be administered for at least about 10 minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject. The T cells may be αβ T cells or γδ T cells that express a CD8 polypeptide described herein, optionally a TCR described herein.

Engineered T-cell expressing a CD8 polypeptides described herein, optionally αβ T cells or γδ T cells, may be present in a composition or pharmaceutical composition in an amount of at least 1×10³ cells/ml, at least 2×10³ cells/ml, at least 3×10³ cells/ml, at least 4×10³ cells/ml, at least 5×10³ cells/ml, at least 6×10³ cells/ml, at least 7×10³ cells/ml, at least 8×10³ cells/ml, at least 9×10³ cells/ml, at least 1×10⁴ cells/ml, at least 2×10⁴ cells/ml, at least 3×10⁴ cells/ml, at least 4×10⁴ cells/ml, at least 5×10⁴ cells/ml, at least 6×10⁴ cells/ml, at least 7×10⁴ cells/ml, at least 8×10⁴ cells/ml, at least 9×10⁴ cells/ml, at least 1×10⁵ cells/ml, at least 2×10⁵ cells/ml, at least 3×10⁵ cells/ml, at least 4×10⁵ cells/ml, at least 5×10⁵ cells/ml, at least 6×10⁵ cells/ml, at least 7×10⁵ cells/ml, at least 8×10⁵ cells/ml, at least 9×10⁵ cells/ml, at least 1×10⁶ cells/ml, at least 2×10⁶ cells/ml, at least 3×10⁶ cells/ml, at least 4×10⁶ cells/ml, at least 5×10⁶ cells/ml, at least 6×10⁶ cells/ml, at least 7×10⁶ cells/ml, at least 8×10⁶ cells/ml, at least 9×10⁶ cells/ml, at least 1×10⁷ cells/ml, at least 2×10⁷ cells/ml, at least 3×10⁷ cells/ml, at least 4×10⁷ cells/ml, at least 5×10⁷ cells/ml, at least 6×10⁷ cells/ml, at least 7×10⁷ cells/ml, at least 8×10⁷ cells/ml, at least 9×10⁷ cells/ml, at least 1×10⁸ cells/ml, at least 2×10⁸ cells/ml, at least 3×10⁸ cells/ml, at least 4×10⁸ cells/ml, at least 5×10⁸ cells/ml, at least 6×10⁸ cells/ml, at least 7×10⁸ cells/ml, at least 8×10⁸ cells/ml, at least 9×10⁸ cells/ml, at least 1×10⁹ cells/ml, or more, from about 1×10³ cells/ml to about at least 1×10⁸ cells/ml, from about 1×10⁵ cells/ml to about at least 1×10⁸ cells/ml, or from about 1×10⁶ cells/ml to about at least 1×10⁸ cells/ml.

### Uses

T cells, T cell populations or pharmaceutical compositions described herein may be used in therapy, in particular in a method of treating cancer. The present disclosure therefore also provides the use of the T cells, T cell populations or pharmaceutical compositions described herein in therapy, in particular in a method of treating cancer. Further, the present disclosure also provides the use of the T cell or population of T cells or the composition described herein in the manufacture of a medicament, in particular a medicament for the treatment of cancer. The cancer may be selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer. The features and aspects described in connection with the methods of treating, preparing and administering above are also applicable to the uses described herein, mutatis mutandis.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

“Activation” as used herein refers broadly to the state of a T cell that has been sufficiently stimulated to induce detectable cellular proliferation. Activation can also be associated with induced cytokine production, and detectable effector functions. The term “activated T cells” refers to, among other things, T cells that are proliferating.

“Antibodies” as used herein refer broadly to antibodies or immunoglobulins of any isotype, fragments of antibodies, which retain specific binding to antigen, including, but not limited to, Fab, Fab′, Fab′-SH, (Fab′)₂ Fv, scFv, divalent scFv, and Fd fragments, chimeric antibodies, humanized antibodies, single-chain antibodies, and fusion proteins including an antigen-specific targeting region of an antibody and a non-antibody protein. Antibodies are organized into five classes—IgG, IgE, IgA, IgD, and IgM.

“Antigen” or “Antigenic,” as used herein, refers broadly to a peptide or a portion of a peptide capable of being bound by an antibody which is additionally capable of inducing an animal to produce an antibody capable of binding to an epitope of that antigen. An antigen may have one epitope or have more than one epitope. The specific reaction referred to herein indicates that the antigen will react, in a highly selective manner, with its corresponding antibody and not with the multitude of other antibodies which may be evoked by other antigens.

“Chimeric antigen receptor” or “CAR” or “CARs” as used herein refers broadly to genetically modified receptors, which graft an antigen specificity onto cells, for example T cells, NK cells, macrophages, and stem cells. CARs can include at least one antigen-specific targeting region (ASTR), a hinge or stalk domain, a transmembrane domain (TM), one or more co-stimulatory domains (CSDs), and an intracellular activating domain (IAD). The CSD may be optional. The CAR may be a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

“Cytotoxic T lymphocyte” (CTL) as used herein refers broadly to a T lymphocyte that expresses CD8 on the surface thereof (e.g., a CD8+ T cell). Such cells may be “memory” T cells (TM cells) that are antigen-experienced.

“Effective amount”, “therapeutically effective amount”, or “efficacious amount” as used herein refers broadly to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

“Genetically modified” as used herein refers broadly to methods to introduce exogenous nucleic acids into a cell, whether or not the exogenous nucleic acids are integrated into the genome of the cell. “Genetically modified cell” as used herein refers broadly to cells that contain exogenous nucleic acids whether or not the exogenous nucleic acids are integrated into the genome of the cell.

“Immune cells” as used herein refers broadly to white blood cells (leukocytes) derived from hematopoietic stem cells (HSC) produced in the bone marrow “Immune cells” include, without limitation, lymphocytes (T cells, B cells, natural killer (NK) (CD3−CD56+) cells) and myeloid-derived cells (neutrophil, eosinophil, basophil, monocyte, macrophage, dendritic cells). “T cells” include all types of immune cells expressing CD3 including T-helper cells (CD4+ cells), cytotoxic T-cells (CD8+ cells), T-regulatory cells (Treg) and gamma-delta T cells, and NK T cells (CD3+ and CD56+). A skilled artisan will understand T cells and/or NK cells, as used throughout the disclosure, can include only T cells, only NK cells, or both T cells and NK cells. T cells may be activated and transduced. Furthermore, T cells are provided in certain illustrative composition embodiments and aspects provided herein. A “cytotoxic cell” includes CD8+ T cells, natural-killer (NK) cells, NK-T cells, γδ T cells, and neutrophils, which are cells capable of mediating cytotoxicity responses.

“Individual,” “subject,” “host,” and “patient,” as used interchangeably herein, refer broadly to a mammal, including, but not limited to, humans, murines (e.g., rats, mice), lagomorphs (e.g., rabbits), non-human primates, canines, felines, and ungulates (e.g., equines, bovines, ovines, porcines, caprines).

“Peripheral blood mononuclear cells” or “PBMCs” as used herein refers broadly to any peripheral blood cell having a round nucleus. PBMCs include lymphocytes, such as T cells, B cells, and NK cells, and monocytes.

“Polynucleotide” and “nucleic acid”, as used interchangeably herein, refer broadly to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases.

“T cell” or “T lymphocyte,” as used herein, refer broadly to thymocytes, naïve T lymphocytes, immature T lymphocytes, mature T lymphocytes, resting T lymphocytes, or activated T lymphocytes. Illustrative populations of T cells suitable for use in particular aspects, methods, uses or treatments include, but are not limited to, helper T cells (HTL; CD4+ T cell), a cytotoxic T cell (CTL; CD8+ T cell), CD4+CD8+ T cell, CD4−CD8− T cell, natural killer T cell, T cells expressing αβ TCR (αβ T cells), T cells expressing γδ TCR (γδ T cells), or any other subset of T cells. Other illustrative populations of T cells suitable for use in particular aspects, methods, uses or treatments include, but are not limited to, T cells expressing one or more of the following markers: CD3, CD4, CD8, CD27, CD28, CD45RA, CD45RO, CD62L, CD127, CD197, and HLA-DR and if desired, can be further isolated by positive or negative selection techniques.

“Treatment,” “treating,” and the like, as used herein refer broadly to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, e.g., arresting its development; and (c) relieving the disease, e.g., causing regression of the disease.

The ability of dendritic cells (DC) to activate and expand antigen-specific CD8+ T cells may depend on the DC maturation stage and that DCs may need to receive a “licensing” signal, associated with IL-12 production, in order to elicit cytolytic immune response. In particular, the provision of signals through CD40 Ligand (CD40L)-CD40 interactions on CD4+ T cells and DCs, respectively, may be considered important for the DC licensing and induction of cytotoxic CD8+ T cells. DC licensing may result in the upregulation of co-stimulatory molecules, increased survival and better cross-presenting capabilities of DCs. This process may be mediated via CD40/CD40L interaction [S. R. Bennet et al., “Help for cytotoxic T-cell responses is mediated by CD40 signalling,” Nature 393(6684):478-480 (1998); S. P. Schoenberger et al., “T-cell help for cytotoxic T-cell help is mediated by CD40-CD40L interactions,” Nature 393(6684):480-483 (1998)], but CD40/CD40L-independent mechanisms also exist (CD70, LTβR). In addition, a direct interaction between CD40L expressed on DCs and CD40 on expressed on CD8+ T-cells has also been suggested, providing a possible explanation for the generation of helper-independent CTL responses [S. Johnson et al., “Selected Toll-like receptor ligands and viruses promote helper-independent cytotoxic T-cell priming by upregulating CD40L on dendritic cells,” Immunity 30(2):218-227 (2009)].

### Example 1

### Exemplary Nucleic Acid and Amino Acid Sequences

TABLE 2
Representative Protein and DNA sequences

| SEQ ID | | |
|---|---|---|
| NO: | Description | Sequence |
| 1 | CD8α Ig-like | SQFRVSPLDRTWNLGETVELKCQVLLSNPTSGCSWLF |
| | domain-1 | QPRGAAASPTFLLYLSQNKPKAAEGLDTQRFSGKRLG |
| | | DTFVLTLSDFRRENEGYYFCSALSNSIMYFSHFVPVFL |
| | | PA |
| 2 | CD8ß region | SVVDFLPTTAQPTKKSTLKKRVCRLPRPETQKGPLCSP |
| 3 | CD8α | IYIWAPLAGTCGVLLLSLVIT |
| | transmembrane | |
| | domain | |
| 4 | CD8α | LYCNHRNRRRVCKCPRPVVKSGDKPSLSARYV |
| | cytoplasmic tail | |
| 5 | m1CD8α (signal- | SQFRVSPLDRTWNLGETVELKCQVLLSNPTSGCSWLF |
| | less) | QPRGAAASPTFLLYLSQNKPKAAEGLDTQRFSGKRLG |
| | | DTFVLTLSDFRRENEGYYFCSALSNSIMYFSHFVPVFL |
| | | PASVVDFLPTTAQPTKKSTLKKRVCRLPRPETQKGPLC |
| | | SPIYIWAPLAGTCGVLLLSLVITLYCNHRNRRRVCKCP |
| | | RPVVKSGDKPSLSARYV |
| 6 | CD8α Signal | MALPVTALLLPLALLLHAARP |
| | peptide | |
| 7 | m1CD8a | MALPVTALLLPLALLLHAARPSQFRVSPLDRTWNLGE |
| | | TVELKCQVLLSNPTSGCSWLFQPRGAAASPTFLLYLS |
| | | QNKPKAAEGLDTQRFSGKRLGDTFVLTLSDFRRENEG |
| | | YYFCSALSNSIMYFSHFVPVFLPASVVDFLPTTAQPTK |
| | | KSTLKKRVCRLPRPETQKGPLCSPIYIWAPLAGTCGVL |
| | | LLSLVITLYCNHRNRRRVCKCPRPVVKSGDKPSLSAR |
| | | YV |
| 8 | CD8β1 | MRPRLWLLLAAQLTVLHGNSVLQQTPAYIKVQTNKM |
| | | VMLSCEAKISLSNMRIYWLRQRQAPSSDSHHEFLALW |
| | | DSAKGTIHGEEVEQEKIAVFRDASRFILNLTSVKPEDS |
| | | GIYFCMIVGSPELTFGKGTQLSVVDFLPTTAQPTKKST |
| | | LKKRVCRLPRPETQKGPLCSPITLGLLVAGVLVLLVSL |
| | | GVAIHLCCRRRRARLRFMKQPQGEGISGTFVPQCLHG |
| | | YYSNTTTSQKLLNPWILKT |
| 9 | CD8β2 | MRPRLWLLLAAQLTVLHGNSVLQQTPAYIKVQTNKM |
| | | VMLSCEAKISLSNMRIYWLRQRQAPSSDSHHEFLALW |
| | | DSAKGTIHGEEVEQEKIAVFRDASRFILNLTSVKPEDS |
| | | GIYFCMIVGSPELTFGKGTQLSVVDFLPTTAQPTKKST |
| | | LKKRVCRLPRPETQKGLKGKVYQEPLSPNACMDTTAI |
| | | LQPHRSCLTHGS |
| 10 | CD8β3 | LQQTPAYIKVQTNKMVMLSCEAKISLSNMRIYWLRQ |
| | | RQAPSSDSHHEFLALWDSAKGTIHGEEVEQEKIAVFR |
| | | DASRFILNLTSVKPEDSGIYFCMIVGSPELTFGKGTQLS |
| | | VVDFLPTTAQPTKKSTLKKRVCRLPRPETQKGPLCSPI |
| | | TLGLLVAGVLVLLVSLGVAIHLCCRRRRARLRFMKQF |
| | | YK |
| 11 | CD8ß4 | LQQTPAYIKVQTNKMVMLSCEAKISLSNMRIYWLRQ |
| | | RQAPSSDSHHEFLALWDSAKGTIHGEEVEQEKIAVFR |
| | | DASRFILNLTSVKPEDSGIYFCMIVGSPELTFGKGTQLS |
| | | VVDFLPTTAQPTKKSTLKKRVCRLPRPETQKGPLCSPI |
| | | TLGLLVAGVLVLLVSLGVAIHLCCRRRRARLRFMKQL |
| | | RLHPLEKCSRMDY |
| 12 | CD8β5 | LQQTPAYIKVQTNKMVMLSCEAKISLSNMRIYWLRQ |
| | | RQAPSSDSHHEFLALWDSAKGTIHGEEVEQEKIAVFR |
| | | DASRFILNLTSVKPEDSGIYFCMIVGSPELTFGKGTQLS |
| | | VVDFLPTTAQPTKKSTLKKRVCRLPRPETQKGPLCSPI |
| | | TLGLLVAGVLVLLVSLGVAIHLCCRRRRARLRFMKQ |
| | | KFNIVCLKISGFTTCCCFQILQISREYGFGVLLQKDIGQ |
| 13 | CD8β6 | LQQTPAYIKVQTNKMVMLSCEAKISLSNMRIYWLRQ |
| | | RQAPSSDSHHEFLALWDSAKGTIHGEEVEQEKIAVFR |
| | | DASRFILNLTSVKPEDSGIYFCMIVGSPELTFGKGTQLS |
| | | VVDFLPTTAQPTKKSTLKKRVCRLPRPETQKGPLCSPI |
| | | TLGLLVAGVLVLLVSLGVAIHLCCRRRRARLRFMKQ |
| | | KFNIVCLKISGFTTCCCFQILQISREYGFGVLLQKDIGQ |
| 14 | CD8β7 | LQQTPAYIKVQTNKMVMLSCEAKISLSNMRIYWLRQ |
| | | RQAPSSDSHHEFLALWDSAKGTIHGEEVEQEKIAVFR |
| | | DASRFILNLTSVKPEDSGIYFCMIVGSPELTFGKGTQLS |
| | | VVDFLPTTAQPTKKSTLKKRVCRLPRPETQKGPLCSPI |
| | | TLGLLVAGVLVLLVSLGVAIHLCCRRRRARLRFMKQP |
| | | QGEGISGTFVPQCLHGYYSNTTTSQKLLNPWILKT |
| 15 | R11KEA alpha | MEKNPLAAPLLILWFHLDCVSSILNVEQSPQSLHVQEG |
| | chain | DSTNFTCSFPSSNFYALHWYRKETAKSPEALFVMTLN |
| | | GDEKKKGRISATLNTKEGYSYLYIKGSQPEDSATYLC |
| | | ALYNNNDMRFGAGTRLTVKPNIQNPDPAVYQLRDSK |
| | | SSDKSVCLFTDFDSQTNVSQSKDSDVYITDKTVLDMR |
| | | SMDFKSNSAVAWSNKSDFACANAFNNSIIPEDTFFPSP |
| | | ESSCDVKLVEKSFETDTNLNFQNLSVIGFRILLLKVAG |
| | | FNLLMTLRLWSS |
| 16 | R11KE beta chain | MDSWTFCCVSLCILVAKHTDAGVIQSPRHEVTEMGQE |
| | | VTLRCKPISGHNSLFWYRETMMRGLELLIYFNNNVPI |
| | | DDSGMPEDRFSAKMPNASFSTLKIQPSEPRDSAVYFC |
| | | ASSPGSTDTQYFGPGTRLTVLEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 17 | R20P1H7 alpha | MEKMLECAFIVLWLQLGWLSGEDQVTQSPEALRLQE |
| | chain | GESSSLNCSYTVSGLRGLFWYRQDPGKGPEFLFTLYS |
| | | AGEEKEKERLKATLTKKESFLHITAPKPEDSATYLCAV |
| | | QGENSGYSTLTFGKGTMLLVSPDIQNPDPAVYQLRDS |
| | | KSSDKSVCLFTDFDSQTNVSQSKDSDVYITDKTVLDM |
| | | RSMDFKSNSAVAWSNKSDFACANAFNNSIIPEDTFFPS |
| | | PESSCDVKLVEKSFETDTNLNFQNLSVIGFRILLLKVA |
| | | GFNLLMTLRLWSS |
| 18 | R20P1H7 beta | MGPQLLGYVVLCLLGAGPLEAQVTQNPRYLITVTGK |
| | chain | KLTVTCSQNMNHEYMSWYRQDPGLGLRQIYYSMNV |
| | | EVTDKGDVPEGYKVSRKEKRNFPLILESPSPNQTSLYF |
| | | CASSLGPGLAAYNEQFFGPGTRLTVLEDLKNVFPPEV |
| | | AVFEPSEAEISHTQKATLVCLATGFYPDHVELSWWVN |
| | | GKEVHSGVSTDPQPLKEQPALNDSRYCLSSRLRVSAT |
| | | FWQNPRNHFRCQVQFYGLSENDEWTQDRAKPVTQIV |
| | | SAEAWGRADCGFTSESYQQGVLSATILYEILLGKATL |
| | | YAVLVSALVLMAMVKRKDSRG |
| 19 | R7P1D5 alpha | MKTFAGFSFLFLWLQLDCMSRGEDVEQSLFLSVREGD |
| | chain | SSVINCTYTDSSSTYLYWYKQEPGAGLQLLTYIFSNM |
| | | DMKQDQRLTVLLNKKDKHLSLRIADTQTGDSAIYFCA |
| | | EYSSASKIIFGSGTRLSIRPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 20 | R7P1D5 beta | MGSWTLCCVSLCILVAKHTDAGVIQSPRHEVTEMGQ |
| | chain | EVTLRCKPISGHDYLFWYRQTMMRGLELLIYFNNNVP |
| | | IDDSGMPEDRFSAKMPNASFSTLKIQPSEPRDSAVYFC |
| | | ASRANTGELFFGEGSRLTVLEDLKNVFPPEVAVFEPSE |
| | | AEISHTQKATLVCLATGFYPDHVELSWWVNGKEVHS |
| | | GVSTDPQPLKEQPALNDSRYCLSSRLRVSATFWQNPR |
| | | NHFRCQVQFYGLSENDEWTQDRAKPVTQIVSAEAWG |
| | | RADCGFTSESYQQGVLSATILYEILLGKATLYAVLVSA |
| | | LVLMAMVKRKDSRG |
| 21 | R10P2G12 alpha | MLTASLLRAVIASICVVSSMAQKVTQAQTEISVVEKE |
| | chain | DVTLDCVYETRDTTYYLFWYKQPPSGELVFLIRRNSF |
| | | DEQNEISGRYSWNFQKSTSSFNFTITASQVVDSAVYFC |
| | | ALSEGNSGNTPLVFGKGTRLSVIANIQNPDPAVYQLR |
| | | DSKSSDKSVCLFTDFDSQTNVSQSKDSDVYITDKTVL |
| | | DMRSMDFKSNSAVAWSNKSDFACANAFNNSIIPEDTF |
| | | FPSPESSCDVKLVEKSFETDTNLNFQNLSVIGFRILLLK |
| | | VAGFNLLMTLRLWSS |
| 22 | R10P2G12 beta | MGIRLLCRVAFCFLAVGLVDVKVTQSSRYLVKRTGE |
| | chain | KVFLECVQDMDHENMFWYRQDPGLGLRLIYFSYDVK |
| | | MKEKGDIPEGYSVSREKKERFSLILESASTNQTSMYLC |
| | | ASSLSSGSHQETQYFGPGTRLLVLEDLKNVFPPEVAVF |
| | | EPSEAEISHTQKATLVCLATGFYPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAE |
| | | AWGRADCGFTSESYQQGVLSATILYEILLGKATLYAV |
| | | LVSALVLMAMVKRKDSRG |
| 23 | R10P1A7 alpha | MKTFAGFSFLFLWLQLDCMSRGEDVEQSLFLSVREGD |
| | chain | SSVINCTYTDSSSTYLYWYKQEPGAGLQLLTYIFSNM |
| | | DMKQDQRLTVLLNKKDKHLSLRIADTQTGDSAIYFCA |
| | | ESKETRLMFGDGTQLVVKPNIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 24 | R10P1A7 beta | MLLLLLLLGPGISLLLPGSLAGSGLGAWSQHPSVWICK |
| | chain | SGTSVKIECRSLDFQATTMFWYRQFPKQSLMLMATSN |
| | | EGSKATYEQGVEKDKFLINHASLTLSTLTVTSAHPEDS |
| | | SFYICSARAGGHEQFFGPGTRLTVLEDLKNVFPPEVAV |
| | | FEPSEAEISHTQKATLVCLATGFYPDHVELSWVWNGK |
| | | EVHSGVSTDPQPLKEQPALNDSRYCLSSRLRVSATFW |
| | | QNPRNHFRCQVQFYGLSENDEWTQDRAKPVTQIVSA |
| | | EAWGRADCGFTSESYQQGVLSATILYEILLGKATLYA |
| | | VLVSALVLMAMVKRKDSRG |
| 25 | R4P1D10 alpha | MKSLRVLLVILWLQLSWVWSQQKEVEQNSGPLSVPE |
| | chain | GAIASLNCTYSDRGSQSFFWYRQYSGKSPELIMFIYSN |
| | | GDKEDGRFTAQLNKASQYVSLLIRDSQPSDSATYLCA |
| | | VNFHDKIIFGKGTRLHILPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 26 | R4P1D10 beta | MGFRLLCCVAFCLLGAGPVDSGVTQTPKHLITATGQR |
| | chain | VTLRCSPRSGDLSVYWYQQSLDQGLQFLIHYYNGEER |
| | | AKGNILERFSAQQFPDLHSELNLSSLELGDSALYFCAS |
| | | SVASAYGYTFGSGTRLTVVEDLNKVFPPEVAVFEPSE |
| | | AEISHTQKATLVCLATGFFPDHVELSWWVNGKEVHS |
| | | GVSTDPQPLKEQPALNDSRYCLSSRLRVSATFWQNPR |
| | | NHFRCQVQFYGLSENDEWTQDRAKPVTQIVSAEAWG |
| | | RADCGFTSVSYQQGVLSATILYEILLGKATLYAVLVS |
| | | ALVLMAMVKRKDF |
| 27 | R4P3F9 alpha | MKSLRVLLVILWLQLSWVWSQQKEVEQNSGPLSVPE |
| | chain | GAIASLNCTYSDRGSQSFFWYRQYSGKSPELIMFIYSN |
| | | GDKEDGRFTAQLNKASQYVSLLIRDSQPSDSATYLCA |
| | | AYSGAGSYQLTFGKGTKLSVIPNIQNPDPAVYQLRDS |
| | | KSSDKSVCLFTDFDSQTNVSQSKDSDVYITDKTVLDM |
| | | RSMDFKSNSAVAWSNKSDFACANAFNNSIIPEDTFFPS |
| | | PESSCDVKLVEKSFETDTNLNFQNLSVIGFRILLLKVA |
| | | GFNLLMTLRLWSS |
| 28 | R4P3F9 beta | MGFRLLCCVAFCLLGAGPVDSGVTQTPKHLITATGQR |
| | chain | VTLRCSPRSGDLSVYWYQQSLDQGLQFLIQYYNGEER |
| | | AKGNILERFSAQQFPDLHSELNLSSLELGDSALYFCAS |
| | | SVESSYGYTFGSGTRLTVVEDLNKVFPPEVAVFEPSEA |
| | | EISHTQKATLVCLATGFFPDHVELSWWVNGKEVHSG |
| | | VSTDPQPLKEQPALNDSRYCLSSRLRVSATFWQNPRN |
| | | HFRCQVQFYGLSENDEWTQDRAKPVTQIVSAEAWGR |
| | | ADCGFTSVSYQQGVLSATILYEILLGKATLYAVLVSAL |
| | | VLMAMVKRKDF |
| 29 | R4P3H3 alpha | MKSLRVLLVILWLQLSWVWSQQKEVEQNSGPLSVPE |
| | chain | GAIASLNCTYSDRGSQSFFWYRQYSGKSPELIMFIYSN |
| | | GDKEDGRFTAQLNKASQYVSLLIRDSQPSDSATYLCA |
| | | VKAGNQFYFGTGTSLTVIPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 30 | R4P3H3 beta | MGTRLLCWVVLGFLGTDHTGAGVSQSPRYKVAKRG |
| | chain | QDVALRCDPISGHVSLFWYQQALGQGPEFLTYFQNEA |
| | | QLDKSGLPSDRFFAERPEGSVSTLKIQRTQQEDSAVYL |
| | | CASSLLTSGGDNEQFFGPGTRLTVLEDLKNVFPPEVA |
| | | VFEPSEAEISHTQKATLVCLATGFYPDHVELSWWVNG |
| | | KEVHSGVSTDPQPLKEQPALNDSRYCLSSRLRVSATF |
| | | WQNPRNHFRCQVQFYGLSENDEWTQDRAKPVTQIVS |
| | | AEAWGRADCGFTSESYQQGVLSATILYEILLGKATLY |
| | | AVLVSALVLMAMVKRKDSRG |
| 31 | R36P3F9 alpha | METLLGVSLVILWLQLARVNSQQGEEDPQALSIQEGE |
| | chain | NATMNCSYKTSINNLQWYRQNSGRGLVHLILIRSNER |
| | | EKHSGRLRVTLDTSKKSSSLLITASRAADTASYFCATV |
| | | SNYQLIWGAGTKLIIKPDIQNPDPAVYQLRDSKSSDKS |
| | | VCLFTDFDSQTNVSQSKDSDVYITDKTVLDMRSMDFK |
| | | SNSAVAWSNKSDFACANAFNNSIIPEDTFFPSPESSCD |
| | | VKLVEKSFETDTNLNFQNLSVIGFRILLLKVAGENLLM |
| | | TLRLWSS |
| 32 | R36P3F9 beta | MGPQLLGYVVLCLLGAGPLEAQVTQNPRYLITVTGK |
| | chain | KLTVTCSQNMNHEYMSWYRQDPGLGLRQIYYSMNV |
| | | EVTDKGDVPEGYKVSRKEKRNFPLILESPSPNQTSLYF |
| | | CASSSTSGGLSGETQYFGPGTRLLVLEDLKNVFPPEVA |
| | | VFEPSEAEISHTQKATLVCLATGFYPDHVELSWWVNG |
| | | KEVHSGVSTDPQPLKEQPALNDSRYCLSSRLRVSATF |
| | | WQNPRNHFRCQVQFYGLSENDEWTQDRAKPVTQIVS |
| | | AEAWGRADCGFTSESYQQGVLSATILYEILLGKATLY |
| | | AVLVSALVLMAMVKRKDSRG |
| 33 | R52P2G11 alpha | MKKHLTTFLVILWLYFYRGNGKNQVEQSPQSLIILEG |
| | chain | KNCTLQCNYTVSPFSNLRWYKQDTGRGPVSLTIMTFS |
| | | ENTKSNGRYTATLDADTKQSSLHITASQLSDSASYICV |
| | | VSAYGKLQFGAGTQVVVTPDIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 34 | R52P2G11 beta | MDSWTFCCVSLCILVAKHTDAGVIQSPRHEVTEMGQE |
| | chain | VTLRCKPISGHNSLFWYRQTMMRGLELLIYFNNNVPI |
| | | DDSGMPEDRFSAKMPNASFSTLKIQPSEPRDSAVYFC |
| | | ASSLGSPDGNQPQHFGDGTRLSILEDLNKVFPPEVAVF |
| | | EPSEAEISHTQKATLVCLATGFFPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAE |
| | | AWGRADCGFTSVSYQQGVLSATILYEILLGKATLYAV |
| | | LVSALVLMAMVKRKDF |
| 35 | R53P2A9 alpha | MACPGFLWALVISTCLEFSMAQTVTQSQPEMSVQEAE |
| | chain | TVTLSCTYDTSESDYYLFWYKQPPSRQMILVIRQEAY |
| | | KQQNATENRFSVNFQKAAKSFSLKISDSQLGDAAMYF |
| | | CAYNSYAGGTSYGKLTFGQGTILTVHPNIQNPDPAVY |
| | | QLRDSKSSDKSVCLFTDFDSQTNVSQSKDSDVYITDK |
| | | TVLDMRSMDFKSNSAVAWSNKSDFACANAFNNSIIPE |
| | | DTFFPSPESSCDVKLVEKSFETDTNLNFQNLSVIGFRIL |
| | | LLKVAGFNLLMTLRLWSS |
| 36 | R53P2A9 beta | MGPGLLCWVLLCLLGAGPVDAGVTQSPTHLIKTRGQ |
| | chain | QVTLRCSPISGHKSVSWYQQVLGQGPQFIFQYYEKEE |
| | | RGRGNFPDRFSARQFPNYSSELNVNALLLGDSALYLC |
| | | ASSLDGTSEQYFGPGTRLTVTEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 37 | R26P1A9 alpha | METLLGVSLVILWLQLARVNSQQGEEDPQALSIQEGE |
| | chain | NATMNCSYKTSINNLQWYRQNSGRGLVHLILIRSNER |
| | | EKHSGRLRVTLDTSKKSSSLLITASRAADTASYFCLIG |
| | | ASGSRLTFGEGTQLTVNPDIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 38 | R26P1A9 beta | MGSWTLCCVSLCILVAKHTDAGVIQSPRHEVTEMGQ |
| | chain | EVTLRCKPISGHDYLFWYRQTMMRGLELLIYFNNNVP |
| | | IDDSGMPEDRFSAKMPNASFSTLKIQPSEPRDSAVYFC |
| | | ASSYFGWNEKLFFGSGTQLSVLEDLNKVFPPEVAVFE |
| | | PSEAEISHTQKATLVCLATGFFPDHVELSWWVNGKEV |
| | | HSGVSTDPQPLKEQPALNDSRYCLSSRLRVSATFWQN |
| | | PRNHFRCQVQFYGLSENDEWTQDRAKPVTQIVSAEA |
| | | WGRADCGFTSVSYQQGVLSATILYEILLGKATLYAVL |
| | | VSALVLMAMVKRKDF |
| 39 | R26P2A6 alpha | MMKSLRVLLVILWLQLSWVWSQQKEVEQDPGPLSVP |
| | chain | EGAIVSLNCTYSNSAFQYFMWYRQYSRKGPELLMYT |
| | | YSSGNKEDGRFTAQVDKSSKYISLFIRDSQPSDSATYL |
| | | CAMSDVSGGYNKLIFGAGTRLAVHPYIQNPDPAVYQL |
| | | RDSKSSDKSVCLFTDFDSQTNVSQSKDSDVYITDKTV |
| | | LDMRSMDFKSNSAVAWSNKSDFACANAFNNSIIPEDT |
| | | FFPSPESSCDVKLVEKSFETDTNLNFQNLSVIGFRILLL |
| | | KVAGFNLLMTLRLWSS |
| 40 | R26P2A6 beta | MGPQLLGYVVLCLLGAGPLEAQVTQNPRYLITVTGK |
| | chain | KLTVTCSQNMNHEYMSWYRQDPGLGLRQIYYSMNV |
| | | EVTDKGDVPEGYKVSRKEKRNFPLILESPSPNQTSLYF |
| | | CASTTPDGTDEQFFGPGTRLTVLEDLKNVFPPEVAVFE |
| | | PSEAEISHTQKATLVCLATGFYPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAE |
| | | AWGRADCGFTSESYQQGVLSATILYEILLGKATLYAV |
| | | LVSALVLMAMVKRKDSRG |
| 41 | R26P3H1 alpha | MASAPISMLAMLFTLSGLRAQSVAQPEDQVNVAEGN |
| | chain | PLTVKCTYSVSGNPYLFWYVQYPNRGLQFLLKYITGD |
| | | NLVKGSYGFEAEFNKSQTSFHLKKPSALVSDSALYFC |
| | | AVRDMNRDDKIIFGKGTRLHILPNIQNPDPAVYQLRDS |
| | | KSSDKSVCLFTDFDSQTNVSQSKDSDVYITDKTVLDM |
| | | RSMDFKSNSAVAWSNKSDFACANAFNNSIIPEDTFFPS |
| | | PESSCDVKLVEKSFETDTNLNFQNLSVIGFRILLLKVA |
| | | GFNLLMTLRLWSS |
| 42 | R26P3H1 beta | MSNQVLCCVVLCFLGANTVDGGITQSPKYLFRKEGQ |
| | chain | NVTLSCEQNLNHDAMYWYRQDPGQGLRLIYYSQIVN |
| | | DFQKGDIAEGYSVSREKKESFPLTVTSAQKNPTAFYLC |
| | | ASSRAEGGEQYFGPGTRLTVTEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 43 | R35P3A4 alpha | MTSIRAVFIFLWLQLDLVNGENVEQHPSTLSVQEGDS |
| | chain | AVIKCTYSDSASNYFPWYKQELGKRPQLIIDIRSNVGE |
| | | KKDQRIAVTLNKTAKHFSLHITETQPEDSAVYFCAASP |
| | | TGGYNKLIFGAGTRLAVHPYIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 44 | R35P3A4 beta | MSIGLLCCAALSLLWAGPVNAGVTQTPKFQVLKTGQ |
| | chain | SMTLQCAQDMNHEYMSWYRQDPGMGLRLIHYSVGA |
| | | GITDQGEVPNGYNVSRSTTEDFPLRLLSAAPSQTSVYF |
| | | CASSLGGASQEQYFGPGTRLTVTEDLKNVFPPEVAVF |
| | | EPSEAEISHTQKATLVCLATGFYPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAE |
| | | AWGRADCGFTSESYQQGVLSATILYEILLGKATLYAV |
| | | LVSALVLMAMVKRKDSRG |
| 45 | R37P1C9 alpha | MKLVTSITVLLSLGIMGDAKTTQPNSMESNEEEPVHLP |
| | chain | CNHSTISGTDYIHWYRQLPSQGPEYVIHGLTSNVNNR |
| | | MASLAIAEDRKSSTLILHRATLRDAAVYYCILFNFNKF |
| | | YFGSGTKLNVKPNIQNPDPAVYQLRDSKSSDKSVCLF |
| | | TDFDSQTNVSQSKDSDVYITDKTVLDMRSMDFKSNSA |
| | | VAWSNKSDFACANAFNNSIIPEDTFFPSPESSCDVKLV |
| | | EKSFETDTNLNFQNLSVIGFRILLLKVAGFNLLMTLRL |
| | | WSS |
| 46 | R37P1C9 beta | MGPGLLHWMALCLLGTGHGDAMVIQNPRYQVTQFG |
| | chain | KPVTLSCSQTLNHNVMYWYQQKSSQAPKLLFHYYDK |
| | | DENNEADTPDNFQSRRPNTSFCFLDIRSPGLGDAAMY |
| | | LCATSSGETNEKLFFGSGTQLSVLEDLNKVFPPEVAVF |
| | | EPSEAEISHTQKATLVCLATGFFPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAE |
| | | AWGRADCGFTSVSYQQGVLSATILYEILLGKATLYAV |
| | | LVSALVLMAMVKRKDF |
| 47 | R37P1H1 alpha | MTRVSLLWAVVVSTCLESGMAQTVTQSQPEMSVQEA |
| | chain | ETVTLSCTYDTSESNYYLFWYKQPPSRQMILVIRQEA |
| | | YKQQNATENRFSVNFQKAAKSFSLKISDSQLGDTAMY |
| | | FCAFGYSGGGADGLTFGKGTHLIIQPYIQNPDPAVYQL |
| | | RDSKSSDKSVCLFTDFDSQTNVSQSKDSDVYITDKTV |
| | | LDMRSMDFKSNSAVAWSNKSDFACANAFNNSIIPEDT |
| | | FFPSPESSCDVKLVEKSFETDTNLNFQNLSVIGFRILLL |
| | | KVAGFNLLMTLRLWSS |
| 48 | R37P1H1 beta | MGPGLLCWALLCLLGAGLVDAGVTQSPTHLIKTRGQ |
| | chain | QVTLRCSPKSGHDTVSWYQQALGQGPQFIFQYYEEEE |
| | | RQRGNFPDRFSGHQFPNYSSELNVNALLLGDSALYLC |
| | | ASSNEGQGWEAEAFFGQGTRLTVVEDLNKVFPPEVA |
| | | VFEPSEAEISHTQKATLVCLATGFFPDHVELSWWVNG |
| | | KEVHSGVSTDPQPLKEQPALNDSRYCLSSRLRVSATF |
| | | WQNPRNHFRCQVQFYGLSENDEWTQDRAKPVTQIVS |
| | | AEAWGRADCGFTSVSYQQGVLSATILYEILLGKATLY |
| | | AVLVSALVLMAMVKRKDF |
| 49 | R42P3A9 alpha | MKRILGALLGLLSAQVCCVRGIQVEQSPPDLILQEGAN |
| | chain | STLRCNFSDSVNNLQWFHQNPWGQLINLFYIPSGTKQ |
| | | NGRLSATTVATERYSLLYISSSQTTDSGVYFCAVHNFN |
| | | KFYFGSGTKLNVKPNIQNPDPAVYQLRDSKSSDKSVC |
| | | LFTDFDSQTNVSQSKDSDVYITDKTVLDMRSMDFKSN |
| | | SAVAWSNKSDFACANAFNNSIIPEDTFFPSPESSCDVK |
| | | LVEKSFETDTNLNFQNLSVIGFRILLLKVAGENLLMTL |
| | | RLWSS |
| 50 | R42P3A9 beta | MLSPDLPDSAWNTRLLCHVMLCLLGAVSVAAGVIQS |
| | chain | PRHLIKEKRETATLKCYPIPRHDTVYWYQQGPGQDPQ |
| | | FLISFYEKMQSDKGSIPDRFSAQQFSDYHSELNMSSLE |
| | | LGDSALYFCASSLLGQGYNEQFFGPGTRLTVLEDLKN |
| | | VFPPEVAVFEPSEAEISHTQKATLVCLATGFYPDHVEL |
| | | SWWVNGKEVHSGVSTDPQPLKEQPALNDSRYCLSSR |
| | | LRVSATFWQNPRNHFRCQVQFYGLSENDEWTQDRAK |
| | | PVTQIVSAEAWGRADCGFTSESYQQGVLSATILYEILL |
| | | GKATLYAVLVSALVLMAMVKRKDSRG |
| 51 | R43P3F2 alpha | MLTASLLRAVIASICVVSSMAQKVTQAQTEISVVEKE |
| | chain | DVTLDCVYETRDTTYYLFWYKQPPSGELVFLIRRNSF |
| | | DEQNEISGRYSWNFQKSTSSFNFTITASQVVDSAVYFC |
| | | ALSNNNAGNMLTFGGGTRLMVKPHIQNPDPAVYQLR |
| | | DSKSSDKSVCLFTDFDSQTNVSQSKDSDVYITDKTVL |
| | | DMRSMDFKSNSAVAWSNKSDFACANAFNNSIIPEDTF |
| | | FPSPESSCDVKLVEKSFETDTNLNFQNLSVIGFRILLLK |
| | | VAGFNLLMTLRLWSS |
| 52 | R43P3F2 beta | MLSPDLPDSAWNTRLLCHVMLCLLGAVSVAAGVIQS |
| | chain | PRHLIKEKRETATLKCYPIPRHDTVYWYQQGPGQDPQ |
| | | FLISFYEKMQSDKGSIPDRFSAQQFSDYHSELNMSSLE |
| | | LGDSALYFCASSPTGTSGYNEQFFGPGTRLTVLEDLK |
| | | NVFPPEVAVFEPSEAEISHTQKATLVCLATGFYPDHVE |
| | | LSWWVNGKEVHSGVSTDPQPLKEQPALNDSRYCLSS |
| | | RLRVSATFWQNPRNHFRCQVQFYGLSENDEWTQDRA |
| | | KPVTQIVSAEAWGRADCGFTSESYQQGVLSATILYEIL |
| | | LGKATLYAVLVSALVLMAMVKRKDSRG |
| 53 | R43P3G5 alpha | MEKNPLAAPLLILWFHLDCVSSILNVEQSPQSLHVQEG |
| | chain | DSTNFTCSFPSSNFYALHWYRWETAKSPEALFVMTLN |
| | | GDEKKKGRISATLNTKEGYSYLYIKGSQPEDSATYLC |
| | | ALNRDDKIIFGKGTRLHILPNIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 54 | R43P3G5 beta | MGIRLLCRVAFCFLAVGLVDVKVTQSSRYLVKRTGE |
| | chain | KVFLECVQDMDHENMFWYRQDPGLGLRLIYFSYDVK |
| | | MKEKGDIPEGYSVSREKKERFSLILESASTNQTSMYLC |
| | | ASRLPSRTYEQYFGPGTRLTVTEDLKNVFPPEVAVFEP |
| | | SEAEISHTQKATLVCLATGFYPDHVELSWWVNGKEV |
| | | HSGVSTDPQPLKEQPALNDSRYCLSSRLRVSATFWQN |
| | | PRNHFRCQVQFYGLSENDEWTQDRAKPVTQIVSAEA |
| | | WGRADCGFTSESYQQGVLSATILYEILLGKATLYAVL |
| | | VSALVLMAMVKRKDSRG |
| 55 | R59P2E7 alpha | METLLGLLILWLQLQWVSSKQEVTQIPAALSVPEGEN |
| | chain | LVLNCSFTDSAIYNLQWFRQDPGKGLTSLLLIQSSQRE |
| | | QTSGRLNASLDKSSGRSTLYIAASQPGDSATYLCAVN |
| | | SDYKLSFGAGTTVTVRANIQNPDPAVYQLRDSKSSDK |
| | | SVCLFTDFDSQTNVSQSKDSDVYITDKTVLDMRSMDF |
| | | KSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 56 | R59P2E7 beta | MLSPDLPDSAWNTRLLCHVMLCLLGAVSVAAGVIQS |
| | chain | PRHLIKEKRETATLKCYPIPRHDTVYWYQQGPGQDPQ |
| | | FLISFYEKMQSDKGSIPDRFSAQQFSDYHSELNMSSLE |
| | | LGDSALYFCASSLGLGTGDYGYTFGSGTRLTVVEDLN |
| | | KVFPPEVAVFEPSEAEISHTQKATLVCLATGFFPDHVE |
| | | LSWWVNGKEVHSGVSTDPQPLKEQPALNDSRYCLSS |
| | | RLRVSATFWQNPRNHFRCQVQFYGLSENDEWTQDRA |
| | | KPVTQIVSAEAWGRADCGFTSVSYQQGVLSATILYEIL |
| | | LGKATLYAVLVSALVLMAMVKRKDF |
| 57 | R11P3D3 alpha | MEKNPLAAPLLILWFHLDCVSSILNVEQSPQSLHVQEG |
| | chain | DSTNFTCSFPSSNFYALHWYRWETAKSPEALFVMTLN |
| | | GDEKKKGRISATLNTKEGYSYLYIKGSQPEDSATYLC |
| | | ALYNNNDMRFGAGTRLTVKPNIQNPDPAVYQLRDSK |
| | | SSDKSVCLFTDFDSQTNVSQSKDSDVYITDKTVLDMR |
| | | SMDFKSNSAVAWSNKSDFACANAFNNSIIPEDTFFPSP |
| | | ESSCDVKLVEKSFETDTNLNFQNLSVIGFRILLLKVAG |
| | | FNLLMTLRLWSS |
| 58 | R11P3D3 beta | MDSWTFCCVSLCILVAKHTDAGVIQSPRHEVTEMGQE |
| | chain | VTLRCKPISGHNSLFWYRQTMMRGLELLIYFNNNVPI |
| | | DDSGMPEDRFSAKMPNASFSTLKIQPSEPRDSAVYFC |
| | | ASSPGSTDTQYFGPGTRLTVLEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 59 | R16P1C10 alpha | MKSLRVLLVILWLQLSWVWSQQKEVEQNSGPLSVPE |
| | chain | GAIASLNCTYSDRGSQSFFWYRQYSGKSPELIMFIYSN |
| | | GDKEDGRFTAQLNKASQYVSLLIRDSQPSDSATYLCA |
| | | AVISNFGNEKLTFGTGTRLTIIPNIQNPDPAVYQLRDSK |
| | | SSDKSVCLFTDFDSQTNVSQSKDSDVYITDKTVLDMR |
| | | SMDFKSNSAVAWSNKSDFACANAFNNSIIPEDTFFPSP |
| | | ESSCDVKLVEKSFETDTNLNFQNLSVIGFRILLLKVAG |
| | | FNLLMTLRLWSS |
| 60 | R16P1C10 beta | MGSRLLCWVLLCLLGAGPVKAGVTQTPRYLIKTRGQ |
| | chain | QVTLSCSPISGHRSVSWYQQTPGQGLQFLFEYFSETQR |
| | | NKGNFPGRFSGRQFSNSRSEMNVSTLELGDSALYLCA |
| | | SSPWDSPNEQYFGPGTRLTVTEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 61 | R16P1E8 alpha | MMKSLRVLLVILWLQLSWVWSQQKEVEQDPGPLSVP |
| | chain | EGAIVSLNCTYSNSAFQYFMWYRQYSRKGPELLMYT |
| | | YSSGNKEDGRFTAQVDKSSKYISLFIRDSQPSDSATYL |
| | | CAMSEAAGNKLTFGGGTRVLVKPNIQNPDPAVYQLR |
| | | DSKSSDKSVCLFTDFDSQTNVSQSKDSDVYITDKTVL |
| | | DMRSMDFKSNSAVAWSNKSDFACANAFNNSIIPEDTF |
| | | FPSPESSCDVKLVEKSFETDTNLNFQNLSVIGFRILLLK |
| | | VAGFNLLMTLRLWSS |
| 62 | R16P1E8 beta | MGTRLLCWAALCLLGAELTEAGVAQSPRYKIIEKRQS |
| | chain | VAFWCNPISGHATLYWYQQILGQGPKLLIQFQNNGVV |
| | | DDSQLPKDRFSAERLKGVDSTLKIQPAKLEDSAVYLC |
| | | ASSYTNQGEAFFGQGTRLTVVEDLNKVFPPEVAVFEP |
| | | SEAEISHTQKATLVCLATGFFPDHVELSWWVNGKEV |
| | | HSGVSTDPQPLKEQPALNDSRYCLSSRLRVSATFWQN |
| | | PRNHFRCQVQFYGLSENDEWTQDRAKPVTQIVSAEA |
| | | WGRADCGFTSVSYQQGVLSATILYEILLGKATLYAVL |
| | | VSALVLMAMVKRKDF |
| 63 | R17P1A9 alpha | MKSLRVLLVILWLQLSWVWSQQKEVEQNSGPLSVPE |
| | chain | GAIASLNCTYSDRGSQSFFWYRQYSGKSPELIMSIYSN |
| | | GDKEDGRFTAQLNKASQYVSLLIRDSQPSDSATYLCA |
| | | VLNQAGTALIFGKGTTLSVSSNIQNPDPAVYQLRDSKS |
| | | SDKSVCLFTDFDSQTNVSQSKDSDVYITDKTVLDMRS |
| | | MDFKSNSAVAWSNKSDFACANAFNNSIIPEDTFFPSPE |
| | | SSCDVKLVEKSFETDTNLNFQNLSVIGFRILLLKVAGF |
| | | NLLMTLRLWSS |
| 64 | R17P1A9 beta | MGFRLLCCVAFCLLGAGPVDSGVTQTPKHLITATGQR |
| | chain | VTLRCSPRSGDLSVYWYQQSLDQGLQFLIQYYNGEER |
| | | AKGNILERFSAQQFPDLHSELNLSSLELGDSALYFCAS |
| | | SAETGPWLGNEQFFGPGTRLTVLEDLKNVFPPEVAVF |
| | | EPSEAEISHTQKATLVCLATGFYPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAE |
| | | AWGRADCGFTSESYQQGVLSATILYEILLGKATLYAV |
| | | LVSALVLMAMVKRKDSRG |
| 65 | R17P1D7 alpha | MACPGFLWALVISTCLEFSMAQTVTQSQPEMSVQEAE |
| | chain | TVTLSCTYDTSESDYYLFWYKQPPSRQMILVIRQEAY |
| | | KQQNATENRFSVNFQKAAKSFSLKISDSQLGDAAMYF |
| | | CAYRWAQGGSEKLVFGKGTKLTVNPYIQKPDPAVYQ |
| | | LRDSKSSDKSVCLFTDFDSQTNVSQSKDSDVYITDKT |
| | | VLDMRSMDFKSNSAVAWSNKSDFACANAFNNSIIPED |
| | | TFFPSPESSCDVKLVEKSFETDTNLNFQNLSVIGFRILL |
| | | LKVAGFNLLMTLRLWSS |
| 66 | R17P1D7 beta | MTIRLLCYMGFYFLGAGLMEADIYQTPRYLVIGTGKK |
| | chain | ITLECSQTMGHDKMYWYQQDPGMELHLIHYSYGVNS |
| | | TEKGDLSSESTVSRIRTEHFPLTLESARPSHTSQYLCAT |
| | | ELWSSGGTGELFFGEGSRLTVLEDLKNVFPPEVAVFEP |
| | | SEAEISHTQKATLVCLATGFYPDHVELSWWVNGKEV |
| | | HSGVSTDPQPLKEQPALNDSRYCLSSRLRVSATFWQN |
| | | PRNHFRCQVQFYGLSENDEWTQDRAKPVTQIVSAEA |
| | | WGRADCGFTSESYQQGVLSATILYEILLGKATLYAVL |
| | | VSALVLMAMVKRKDSRG |
| 67 | R17P1G3 alpha | MKSLRVLLVILWLQLSWVWSQQKEVEQNSGPLSVPE |
| | chain | GAIASLNCTYSDRGSQSFFWYRQYSGKSPELIMSIYSN |
| | | GDKEDGRFTAQLNKASQYVSLLIRDSQPSDSATYLCA |
| | | VGPSGTYKYIFGTGTRLKVLANIQNPDPAVYQLRDSK |
| | | SSDKSVCLFTDFDSQTNVSQSKDSDVYITDKTVLDMR |
| | | SMDFKSNSAVAWSNKSDFACANAFNNSIIPEDTFFPSP |
| | | ESSCDVKLVEKSFETDTNLNFQNLSVIGFRILLLKVAG |
| | | FNLLMTLRLWSS |
| 68 | R17P1G3 beta | MGPQLLGYVVLCLLGAGPLEAQVTQNPRYLITVTGK |
| | chain | KLTVTCSQNMNHEYMSWYRQDPGLGLRQIYYSMNV |
| | | EVTDKGDVPEGYKVSRKEKRNFPLILESPSPNQTSLYF |
| | | CASSPGGSGNEQFFGPGTRLTVLEDLKNVFPPEVAVFE |
| | | PSEAEISHTQKATLVCLATGFYPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAE |
| | | AWGRADCGFTSESYQQGVLSATILYEILLGKATLYAV |
| | | LVSALVLMAMVKRKDSRG |
| 69 | R17P2B6 alpha | MKSLRVLLVILWLQLSWVWSQQKEVEQNSGPLSVPE |
| | chain | GAIASLNCTYSDRGSQSFFWYRQYSGKSPELIMFIYSN |
| | | GDKEDGRFTAQLNKASQYVSLLIRDSQPSDSATYLCA |
| | | VVSGGGADGLTFGKGTHLIIQPYIQKPDPAVYQLRDS |
| | | KSSDKSVCLFTDFDSQTNVSQSKDSDVYITDKTVLDM |
| | | RSMDFKSNSAVAWSNKSDFACANAFNNSIIPEDTFFPS |
| | | PESSCDVKLVEKSFETDTNLNFQNLSVIGFRILLLKVA |
| | | GFNLLMTLRLWSS |
| 70 | R17P2B6 beta | MLSPDLPDSAWNTRLLCHVMLCLLGAVSVAAGVIQS |
| | chain | PRHLIKEKRETATLKCYPIPRHDTVYWYQQGPGQDPQ |
| | | FLISFYEKMQSDKGSIPDRFSAQQFSDYHSELNMSSLE |
| | | LGDSALYFCASSLGRGGQPQHFGDGTRLSILEDLNKV |
| | | FPPEVAVFEPSEAEISHTQKATLVCLATGFFPDHVELS |
| | | WWVNGKEVHSGVSTDPQPLKEQPALNDSRYCLSSRL |
| | | RVSATFWQNPRNHFRCQVQFYGLSENDEWTQDRAKP |
| | | VTQIVSAEAWGRADCGFTSVSYQQGVLSATILYEILLG |
| | | KATLYAVLVSALVLMAMVKRKDF |
| 71 | R11P3D3KE | MEKNPLAAPLLILWFHLDCVSSILNVEQSPQSLHVQEG |
| | alpha chain | DSTNFTCSFPSSNFYALHWYRKETAKSPEALFVMTLN |
| | | GDEKKKGRISATLNTKEGYSYLYIKGSQPEDSATYLC |
| | | ALYNNNDMRFGAGTRLTVKPNIQNPDPAVYQLRDSK |
| | | SSDKSVCLFTDFDSQTNVSQSKDSDVYITDKTVLDMR |
| | | SMDFKSNSAVAWSNKSDFACANAFNNSIIPEDTFFPSP |
| | | ESSCDVKLVEKSFETDTNLNFQNLSVIGFRILLLKVAG |
| | | FNLLMTLRLWSS |
| 72 | R11P3D3KE beta | MDSWTFCCVSLCILVAKHTDAGVIQSPRHEVTEMGQE |
| | chain | VTLRCKPISGHNSLFWYRETMMRGLELLIYFNNNVPI |
| | | DDSGMPEDRFSAKMPNASFSTLKIQPSEPRDSAVYFC |
| | | ASSPGSTDTQYFGPGTRLTVLEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 73 | R39P1C12 alpha | MKTFAGFSFLFLWLQLDCMSRGEDVEQSLFLSVREGD |
| | chain | SSVINCTYTDSSSTYLYWYKQEPGAGLQLLTYIFSNM |
| | | DMKQDQRLTVLLNKKDKHLSLRIADTQTGDSAIYFCA |
| | | EIDNQGGKLIFGQGTELSVKPNIQNPDPAVYQLRDSKS |
| | | SDKSVCLFTDFDSQTNVSQSKDSDVYITDKTVLDMRS |
| | | MDFKSNSAVAWSNKSDFACANAFNNSIIPEDTFFPSPE |
| | | SSCDVKLVEKSFETDTNLNFQNLSVIGFRILLLKVAGF |
| | | NLLMTLRLWSS |
| 74 | R39P1C12 beta | MGPGLLCWALLCLLGAGLVDAGVTQSPTHLIKTRGQ |
| | chain | QVTLRCSPKSGHDTVSWYQQALGQGPQFIFQYYEEEE |
| | | RQRGNFPDRFSGHQFPNYSSELNVNALLLGDSALYLC |
| | | ASSQLNTEAFFGQGTRLTVVEDLNKVFPPEVAVFEPSE |
| | | AEISHTQKATLVCLATGFFPDHVELSWWVNGKEVHS |
| | | GVSTDPQPLKEQPALNDSRYCLSSRLRVSATFWQNPR |
| | | NHFRCQVQFYGLSENDEWTQDRAKPVTQIVSAEAWG |
| | | RADCGFTSVSYQQGVLSATILYEILLGKATLYAVLVS |
| | | ALVLMAMVKRKDF |
| 75 | R39P1F5 alpha | MKSLRVLLVILWLQLSWVWSQQKEVEQNSGPLSVPE |
| | chain | GAIASLNCTYSDRGSQSFFWYRQYSGKSPELIMFIYSN |
| | | GDKEDGRFTAQLNKASQYVSLLIRDSQPSDSATYLCA |
| | | VNNARLMFGDGTQLVVKPNIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 76 | R39P1F5 beta | MDTWLVCWAIFSLLKAGLTEPEVTQTPSHQVTQMGQ |
| | chain | EVILRCVPISNHLYFYWYRQILGQKVEFLVSFYNNEIS |
| | | EKSEIFDDQFSVERPDGSNFTLKIRSTKLEDSAMYFCA |
| | | SSGQGANEQYFGPGTRLTVTEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 77 | R40P1C2 alpha | MACPGFLWALVISTCLEFSMAQTVTQSQPEMSVQEAE |
| | chain | TVTLSCTYDTSESDYYLFWYKQPPSRQMILVIRQEAY |
| | | KQQNATENRFSVNFQKAAKSFSLKISDSQLGDAAMYF |
| | | CAYLNYQLIWGAGTKLIIKPDIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 78 | R40P1C2 beta | MDTWLVCWAIFSLLKAGLTEPEVTQTPSHQVTQMGQ |
| | chain | EVILRCVPISNHLYFYWYRQILGQKVEFLVSFYNNEIS |
| | | EKSEIFDDQFSVERPDGSNFTLKIRSTKLEDSAMYFCA |
| | | SSEMTAVGQYFGPGTRLTVTEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 79 | R41P3E6 alpha | MKSLRVLLVILWLQLSWVWSQQKEVEQNSGPLSVPE |
| | chain | GAIASLNCTYSDRGSQSFFWYRQYSGKSPELIMFIYSN |
| | | GDKEDGRFTAQLNKASQYVSLLIRDSQPSDSATYLCA |
| | | AFSGYALNFGKGTSLLVTPHIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 80 | R41P3E6 beta | MDTWLVCWAIFSLLKAGLTEPEVTQTPSHQVTQMGQ |
| | chain | EVILRCVPISNHLYFYWYRQILGQKVEFLVSFYNNEIS |
| | | EKSEIFDDQFSVERPDGSNFTLKIRSTKLEDSAMYFCA |
| | | SSQYTGELFFGEGSRLTVLEDLKNVFPPEVAVFEPSEA |
| | | EISHTQKATLVCLATGFYPDHVELSWWVNGKEVHSG |
| | | VSTDPQPLKEQPALNDSRYCLSSRLRVSATFWQNPRN |
| | | HFRCQVQFYGLSENDEWTQDRAKPVTQIVSAEAWGR |
| | | ADCGFTSESYQQGVLSATILYEILLGKATLYAVLVSAL |
| | | VLMAMVKRKDSRG |
| 81 | R43P3G4 alpha | MKSLRVLLVILWLQLSWVWSQQKEVEQNSGPLSVPE |
| | chain | GAIASLNCTYSDRGSQSFFWYRQYSGKSPELIMFIYSN |
| | | GDKEDGRFTAQLNKASQYVSLLIRDSQPSDSATYLCA |
| | | VNGGDMRFGAGTRLTVKPNIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 82 | R43P3G4 beta | MDTWLVCWAIFSLLKAGLTEPEVTQTPSHQVTQMGQ |
| | chain | EVILRCVPISNHLYFYWYRQILGQKVEFLVSFYNNEIS |
| | | EKSEIFDDQFSVERPDGSNFTLKIRSTKLEDSAMYFCA |
| | | SSGQGALEQYFGPGTRLTVTEDLKNVFPPEVAVFEPSE |
| | | AEISHTQKATLVCLATGFYPDHVELSWWVNGKEVHS |
| | | GVSTDPQPLKEQPALNDSRYCLSSRLRVSATFWQNPR |
| | | NHFRCQVQFYGLSENDEWTQDRAKPVTQIVSAEAWG |
| | | RADCGFTSESYQQGVLSATILYEILLGKATLYAVLVSA |
| | | LVLMAMVKRKDSRG |
| 83 | R44P3B3 alpha | MAMLLGASVLILWLQPDWVNSQQKNDDQQVKQNSP |
| | chain | SLSVQEGRISILNCDYTNSMFDYFLWYKKYPAEGPTFL |
| | | ISISSIKDKNEDGRFTVFLNKSAKHLSLHIVPSQPGDSA |
| | | VYFCAASGLYNQGGKLIFGQGTELSVKPNIQNPDPAV |
| | | YQLRDSKSSDKSVCLFTDFDSQTNVSQSKDSDVYITD |
| | | KTVLDMRSMDFKSNSAVAWSNKSDFACANAFNNSIIP |
| | | EDTFFPSPESSCDVKLVEKSFETDTNLNFQNLSVIGFRI |
| | | LLLKVAGFNLLMTLRLWSS |
| 84 | R44P3B3 beta | MGCRLLCCVVFCLLQAGPLDTAVSQTPKYLVTQMGN |
| | chain | DKSIKCEQNLGHDTMYWYKQDSKKFLKIMFSYNNKE |
| | | LIINETVPNRFSPKSPDKAHLNLHINSLELGDSAVYFCA |
| | | SSLGDRGYEQYFGPGTRLTVTEDLKNVFPPEVAVFEPS |
| | | EAEISHTQKATLVCLATGFYPDHVELSWWVNGKEVH |
| | | SGVSTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 85 | R44P3E7 alpha | MKTFAGFSFLFLWLQLDCMSRGEDVEQSLFLSVREGD |
| | chain | SSVINCTYTDSSSTYLYWYKQEPGAGLQLLTYIFSNM |
| | | DMKQDQRLTVLLNKKDKHLSLRIADTQTGDSAIYFCA |
| | | EINNNARLMFGDGTQLVVKPNIQNPDPAVYQLRDSKS |
| | | SDKSVCLFTDFDSQTNVSQSKDSDVYITDKTVLDMRS |
| | | MDFKSNSAVAWSNKSDFACANAFNNSIIPEDTFFPSPE |
| | | SSCDVKLVEKSFETDTNLNFQNLSVIGFRILLLKVAGF |
| | | NLLMTLRLWSS |
| 86 | R44P3E7 beta | MLSPDLPDSAWNTRLLCHVMLCLLGAVSVAAGVIQS |
| | chain | PRHLIKEKRETATLKCYPIPRHDTVYWYQQGPGQDPQ |
| | | FLISFYEKMQSDKGSIPDRFSAQQFSDYHSELNMSSLE |
| | | LGDSALYFCASSPPDQNTQYFGPGTRLTVLEDLKNVF |
| | | PPEVAVFEPSEAEISHTQKATLVCLATGFYPDHVELSW |
| | | WVNGKEVHSGVSTDPQPLKEQPALNDSRYCLSSRLR |
| | | VSATFWQNPRNHFRCQVQFYGLSENDEWTQDRAKPV |
| | | TQIVSAEAWGRADCGFTSESYQQGVLSATILYEILLGK |
| | | ATLYAVLVSALVLMAMVKRKDSRG |
| 87 | R49P2B7 alpha | MLLLLVPVLEVIFTLGGTRAQSVTQLGSHVSVSEGAL |
| | chain | VLLRCNYSSSVPPYLFWYVQYPNQGLQLLLKYTTGAT |
| | | LVKGINGFEAEFKKSETSFHLTKPSAHMSDAAEYFCA |
| | | VRIFGNEKLTFGTGTRLTIIPNIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGFNL |
| | | LMTLRLWSS |
| 88 | R49P2B7 beta | MGIRLLCRVAFCFLAVGLVDVKVTQSSRYLVKRTGE |
| | chain | KVFLECVQDMDHENMFWYRQDPGLGLRLIYFSYDVK |
| | | MKEKGDIPEGYSVSREKKERFSLILESASTNQTSMYLC |
| | | ASSLMGELTGELFFGEGSRLTVLEDLKNVFPPEVAVFE |
| | | PSEAEISHTQKATLVCLATGFYPDHVELSWWVNGKE |
| | | VHSGVSTDPQPLKEQPALNDSRYCLSSRLRVSATFWQ |
| | | NPRNHFRCQVQFYGLSENDEWTQDRAKPVTQIVSAE |
| | | AWGRADCGFTSESYQQGVLSATILYEILLGKATLYAV |
| | | LVSALVLMAMVKRKDSRG |
| 89 | R55P1G7 alpha | MMKSLRVLLVILWLQLSWVWSQQKEVEQDPGPLSVP |
| | chain | EGAIVSLNCTYSNSAFQYFMWYRQYSRKGPELLMYT |
| | | YSSGNKEDGRFTAQVDKSSKYISLFIRDSQPSDSATYL |
| | | CAMMGDTGTASKLTFGTGTRLQVTLDIQNPDPAVYQ |
| | | LRDSKSSDKSVCLFTDFDSQTNVSQSKDSDVYITDKT |
| | | VLDMRSMDFKSNSAVAWSNKSDFACANAFNNSIIPED |
| | | TFFPSPESSCDVKLVEKSFETDTNLNFQNLSVIGFRILL |
| | | LKVAGFNLLMTLRLWSS |
| 90 | R55P1G7 beta | MGIRLLCRVAFCFLAVGLVDVKVTQSSRYLVKRTGE |
| | chain | KVFLECVQDMDHENMFWYRQDPGLGLRLIYFSYDVK |
| | | MKEKGDIPEGYSVSREKKERFSLILESASTNQTSMYLC |
| | | ASSFGGYEQYFGPGTRLTVTEDLKNVFPPEVAVFEPSE |
| | | AEISHTQKATLVCLATGFYPDHVELSWWVNGKEVHS |
| | | GVSTDPQPLKEQPALNDSRYCLSSRLRVSATFWQNPR |
| | | NHFRCQVQFYGLSENDEWTQDRAKPVTQIVSAEAWG |
| | | RADCGFTSESYQQGVLSATILYEILLGKATLYAVLVSA |
| | | LVLMAMVKRKDSRG |
| 91 | R59P2A7 alpha | MKSLRVLLVILWLQLSWVWSQQKEVEQNSGPLSVPE |
| | chain | GAIASLNCTYSDRGSQSFFWYRQYSGKSPELIMSIYSN |
| | | GDKEDGRFTAQLNKASQYVSLLIRDSQPSDSATYLCA |
| | | VQPHDMRFGAGTRLTVKPNIQNPDPAVYQLRDSKSSD |
| | | KSVCLFTDFDSQTNVSQSKDSDVYITDKTVLDMRSMD |
| | | FKSNSAVAWSNKSDFACANAFNNSIIPEDTFFPSPESSC |
| | | DVKLVEKSFETDTNLNFQNLSVIGFRILLLKVAGFNLL |
| | | MTLRLWSS |
| 92 | R59P2A7 beta | MLCSLLALLLGTFFGVRSQTIHQWPATLVQPVGSPLSL |
| | chain | ECTVEGTSNPNLYWYRQAAGRGLQLLFYSVGIGQISS |
| | | EVPQNLSASRPQDRQFILSSKKLLLSDSGFYLCAWSGL |
| | | VAEQFFGPGTRLTVLEDLKNVFPPEVAVFEPSEAEISH |
| | | TQKATLVCLATGFYPDHVELSWWVNGKEVHSGVSTD |
| | | PQPLKEQPALNDSRYCLSSRLRVSATFWQNPRNHFRC |
| | | QVQFYGLSENDEWTQDRAKPVTQIVSAEAWGRADCG |
| | | FTSESYQQGVLSATILYEILLGKATLYAVLVSALVLMA |
| | | MVKRKDSRG |
| 93 | P2A | ATNFSLLKQAGDVEENPGP |
| 94 | T2A | EGRGSLLTCGDVEENPGP |
| 95 | E2A | QCTNYALLKLAGDVESNPGP |
| 96 | F2A | VKQTLNFDLLKLAGDVESNPGP |
| 97 | RD114TR | MKLPTGMVILCSLIIVRAGFDDPRKAIALVQKQHGKP |
| | | CECSGGQVSEAPPNSIQQVTCPGKTAYLMTNQKWKC |
| | | RVTPKISPSGGELQNCPCNTFQDSMHSSCYTEYRQCR |
| | | RINKTYYTATLLKIRSGSLNEVQILQNPNQLLQSPCRG |
| | | SINQPVCWSATAPIHISDGGGPLDTKRVWTVQKRLEQI |
| | | HKAMTPELQYHPLALPKVRDDLSLDARTFDILNTTFR |
| | | LLQMSNFSLAQDCWLCLKLGTPTPLAIPTPSLTYSLAD |
| | | SLANASCQIIPPLLVQPMQFSNSSCLSSPFINDTEQIDLG |
| | | AVTFTNCTSVANVSSPLCALNGSVFLCGNNMAYTYLP |
| | | QNWTRLCVQASLLPDIDINPGDEPVPIPAIDHYIHRPKR |
| | | AVQFIPLLAGLGITAAFTTGATGLGVSVTQYTKLSHQL |
| | | ISDVQVLSGTIQDLQDQVDSLAEVVLQNRRGLDLLTA |
| | | EQGGICLALQEKCCFYANKSGIVRNKIRTLQEELQKRR |
| | | ESLASNPLWTGLQGFLPYLLPLLGPLLTLLLILTIGPCV |
| | | FNRLVQFVKDRISVVQALVLTQQYHQLKPL |
| 256 | WPREmut1 | cagtctgacgtacgcgtaatcaacctctggattacaaaatttgtgaaagattgactggta |
| | | ttcttaactatgttgctccttttacgctatgtggatacgctgctttaatgcctttgtatcatgc |
| | | tattgcttcccgtatggctttcattttctcctccttgtataaatcctggttgctgtctctttatg |
| | | aggagttgtggcccgttgtcaggcaacgtggcgtggtgtgcactgtgtttgctgacgc |
| | | aacccccactggttggggcattgccaccacctgtcagctcctttccgggactttcgcttt |
| | | ccccctccctattgccacggcggaactcatcgccgcctgccttgcccgctgctggac |
| | | aggggctcggctgttgggcactgacaattccgtggtgttgtcggggaaatcatcgtcc |
| | | tttccttggctgctcgcctgtgttgccacctggattctgcgcgggacgtccttctgctac |
| | | gtcccttcggccctcaatccagcggaccttccttcccgcggcctgctgccggctctgc |
| | | ggcctcttccgcgtcttcgccttcgccctcagacgagtcggatctccctttgggccgcc |
| | | tccccgcc |
| 257 | WPREmut2 | Gagcatcttaccgccatttatacccatatttgttctgtttttcttgatttgggtatacatttaa |
| | | atgttaataaaacaaaatggtggggcaatcatttacattttttgggatatgtaattactagt |
| | | tcaggtgtattgccacaagacaaacttgttaagaaactttcccgttatttacgctctgttcc |
| | | tgttaatcaacctctggattacaaaatttgtgaaagattgactgatattcttaactttgttgc |
| | | tccttttacgctgtgtggatttgctgctttattgcctctgtatcttgctattgcttcccgtacg |
| | | gctttcgttttctcctccttgtataaatcctggttgctgtctctttttgaggagttgtggccc |
| | | gttgtccgtcaacgtggcgtggtgtgctctgtgtttgctgacgcaacccccactggctg |
| | | gggcattgccaccacctgtcaactcctttctgggactttcgctttccccctcccgatcgc |
| | | cacggcagaactcatcgccgcctgccttgcccgctgctggacaggggctaggttgct |
| | | gggcactgataattccgtggtgttgtc |
| 258 | CD8α1 | MALPVTALLLPLALLLHAARPSQFRVSPLDRTWNLGE |
| | | TVELKCQVLLSNPTSGCSWLFQPRGAAASPTFLLYLS |
| | | QNKPKAAEGLDTQRFSGKRLGDTFVLTLSDFRRENEG |
| | | YYFCSALSNSIMYFSHFVPVFLPAKPTTTPAPRPPTPAP |
| | | TIASQPLSLRPEACRPAAGGAVHTRGLDFACDIYIWAP |
| | | LAGTCGVLLLSLVITLYCNHRNRRRVCKCPRPVVKSG |
| | | DKPSLSARYV |
| 259 | CD8α2 | MALPVTALLLPLALLLHAARPSQFRVSPLDRTWNLGE |
| | | TVELKCQVLLSNPTSGCSWLFQPRGAAASPTFLLYLS |
| | | QNKPKAAEGLDTQRFSGKRLGDTFVLTLSDFRRENEG |
| | | CYFCSALSNSIMYFSHFVPVFLPAKPTTTPAPRPPTPAP |
| | | TIASQPLSLRPEACRPAAGGAVHTRGLDFACDIYIWAP |
| | | LAGTCGVLLLSLVITLYCNHRNRRRVCKCPRPVVKSG |
| | | DKPSLSARYV |
| 260 | CD8α stalk | KPTTTPAPRPPTPAPTIASQPLSLRPEACRPAAGGAVHT |
| | | RGLDFACD |
| 261 | CD8α Ig-like | SQFRVSPLDRTWNLGETVELKCQVLLSNPTSGCSWLF |
| | domain-2 | QPRGAAASPTFLLYLSQNKPKAAEGLDTQRFSGKRLG |
| | | DTFVLTLSDFRRENEGCYFCS2ALSNSIMYFSHFVPVF |
| | | LPA |
| 262 | m2CD8α | MALPVTALLLPLALLLHAARPSQFRVSPLDRTWNLGE |
| | | TVELKCQVLLSNPTSGCSWLFQPRGAAASPTFLLYLS |
| | | QNKPKAAEGLDTQRFSGKRLGDTFVLTLSDFRRENEG |
| | | CYFCSALSNSIMYFSHFVPVFLPASVVDFLPTTAQPTK |
| | | KSTLKKRVCRLPRPETQKGPLCSPIYIWAPLAGTCGVL |
| | | LLSLVITLYCNHRNRRRVCKCPRPVVKSGDKPSLSAR |
| | | YV |
| 263 | MSCV promoter | Tgaaagaccccacctgtaggtttggcaagctagcttaagtaacgccattttgcaaggc |
| | | atggaaaatacataactgagaatagagaagttcagatcaaggttaggaacagagaga |
| | | cagcagaatatgggccaaacaggatatctgtggtaagcagttcctgccccggctcag |
| | | ggccaagaacagatggtccccagatgcggtcccgccctcagcagtttctagagaac |
| | | catcagatgtttccagggtgccccaaggacctgaaaatgaccctgtgccttatttgaac |
| | | taaccaatcagttcgcttctcgcttctgttcgcgcgcttctgctccccgagctcaataaa |
| | | agagcccacaacccctcact |
| 264 | WPRE | cagtctgacgtacgcgtaatcaacctctggattacaaaatttgtgaaagattgactggta |
| | | ttcttaactatgttgctccttttacgctatgtggatacgctgctttaatgcctttgtatcatgc |
| | | tattgcttcccgtatggctttcattttctcctccttgtataaatcctggttgctgtctctttatg |
| | | aggagttgtggcccgttgtcaggcaacgtggcgtggtgtgcactgtgtttgctgacgc |
| | | aacccccactggttggggcattgccaccacctgtcagctcctttccgggactttcgcttt |
| | | ccccctccctattgccacggcggaactcatcgccgcctgccttgcccgctgctggac |
| | | aggggctcggctgttgggcactgacaattccgtggtgttgtcggggaagctgacgtc |
| | | ctttccatggctgctcgcctgtgttgccacctggattctgcgcgggacgtccttctgcta |
| | | cgtcccttcggccctcaatccagcggaccttccttcccgcggcctgctgccggctctg |
| | | cggcctcttccgcgtcttcgccttcgccctcagacgagtcggatctccctttgggccgc |
| | | ctccccgcc |
| 265 | Furin consensus | RXXR |
| 266 | Linker | SGSG |
| 293 | CD8ß Signal | MRPRLWLLLAAQLTVLHGNSV |
| | peptide | |
| 294 | S19 Signal | MEFGLSWLFLVAILKGVQC |
| | peptide | |

### Tumor Associated Antigens (TAA)

In the MHC class I dependent immune reaction, peptides not only have to be able to bind to certain MHC class I molecules expressed by tumor cells, they subsequently also have to be recognized by T cells bearing specific T cell receptors (TCR).

For proteins to be recognized by T-lymphocytes as tumor-specific or -associated antigens, and to be used in a therapy, particular prerequisites must be fulfilled. The antigen should be expressed mainly by tumor cells and not, or in comparably small amounts, by normal healthy tissues. The peptide may be over-presented by tumor cells as compared to normal healthy tissues. It is furthermore desirable that the respective antigen is not only present in a type of tumor, but also in high concentrations (e.g., copy numbers of the respective peptide per cell). Tumor-specific and tumor-associated antigens are often derived from proteins directly involved in transformation of a normal cell to a tumor cell due to their function, e.g., in cell cycle control or suppression of apoptosis. Additionally, downstream targets of the proteins directly causative for a transformation may be up-regulated and thus may be indirectly tumor-associated. Such indirect tumor-associated antigens may also be targets of a vaccination approach. Singh-Jasuja et al. Cancer Immunol. Immunother. 53 (2004): 187-195. Epitopes are present in the amino acid sequence of the antigen, making the peptide an “immunogenic peptide”, and being derived from a tumor associated antigen, leads to a T-cell-response, both in vitro and in vivo.

Any peptide able to bind an MHC molecule may function as a T-cell epitope. For the induction of a T-cell-response, the TAA must be presented to a T cell having a corresponding TCR and the host must not have immunological tolerance for this particular epitope. Exemplary Tumor Associated Antigens (TAA) that may be used with the CD8 polypeptides described herein are disclosed herein.

TABLE 3
TAA Peptide sequences

| SEQ | Amino Acid |
|---|---|
| ID NO: | Sequence |
| 98 | YLYDSETKNA |
| 99 | HLMDQPLSV |
| 100 | GLLKKINSV |
| 101 | FLVDGSSAL |
| 102 | FLFDGSANLV |
| 103 | FLYKIIDEL |
| 104 | FILDSAETTTL |
| 105 | SVDVSPPKV |
| 106 | VADKIHSV |
| 107 | IVDDLTINL |
| 108 | GLLEELVTV |
| 109 | TLDGAAVNQV |
| 110 | SVLEKEIYSI |
| 111 | LLDPKTIFL |
| 112 | YTFSGDVQL |
| 113 | YLMDDFSSL |
| 114 | KVWSDVTPL |
| 115 | LLWGHPRVALA |
| 116 | KIWEELSVLEV |
| 117 | LLIPFTIFM |
| 118 | FLIENLLAA |
| 119 | LLWGHPRVALA |
| 120 | FLLEREQLL |
| 121 | SLAETIFIV |
| 122 | TLLEGISRA |
| 123 | KIQEILTQV |
| 124 | VIFEGEPMYL |
| 125 | SLFESLEYL |
| 126 | SLLNQPKAV |
| 127 | GLAEFQENV |
| 128 | KLLAVIHEL |
| 129 | TLHDQVHLL |
| 130 | TLYNPERTITV |
| 131 | KLQEKIQEL |
| 132 | SVLEKEIYSI |
| 133 | RVIDDSLVVGV |
| 134 | VLFGELPAL |
| 135 | GLVDIMVHL |
| 136 | FLNAIETAL |
| 137 | ALLQALMEL |
| 138 | ALSSSQAEV |
| 139 | SLITGQDLLSV |
| 140 | QLIEKNWLL |
| 141 | LLDPKTIFL |
| 142 | RLHDENILL |
| 143 | YTFSGDVQL |
| 144 | GLPSATTTV |
| 145 | GLLPSAESIKL |
| 146 | KTASINQNV |
| 147 | SLLQHLIGL |
| 148 | YLMDDFSSL |
| 149 | LMYPYIYHV |
| 150 | KVWSDVTPL |
| 151 | LLWGHPRVALA |
| 152 | VLDGKVAVV |
| 153 | GLLGKVTSV |
| 154 | KMISAIPTL |
| 155 | GLLETTGLLAT |
| 156 | TLNTLDINL |
| 157 | VIIKGLEEI |
| 158 | YLEDGFAYV |
| 159 | KIWEELSVLEV |
| 160 | LLIPFTIFM |
| 161 | ISLDEVAVSL |
| 162 | KISDFGLATV |
| 163 | KLIGNIHGNEV |
| 164 | ILLSVLHQL |
| 165 | LDSEALLTL |
| 166 | VLQENSSDYQSNL |
| 167 | HLLGEGAFAQV |
| 168 | SLVENIHVL |
| 169 | YTFSGDVQL |
| 170 | SLSEKSPEV |
| 171 | AMFPDTIPRV |
| 172 | FLIENLLAA |
| 173 | FTAEFLEKV |
| 174 | ALYGNVQQV |
| 175 | LFQSRIAGV |
| 176 | ILAEEPIYIRV |
| 177 | FLLEREQLL |
| 178 | LLLPLELSLA |
| 179 | SLAETIFIV |
| 180 | AILNVDEKNQV |
| 181 | RLFEEVLGV |
| 182 | YLDEVAFML |
| 183 | KLIDEDEPLFL |
| 184 | KLFEKSTGL |
| 185 | SLLEVNEASSV |
| 186 | GVYDGREHTV |
| 187 | GLYPVTLVGV |
| 188 | ALLSSVAEA |
| 189 | TLLEGISRA |
| 190 | SLIEESEEL |
| 191 | ALYVQAPTV |
| 192 | KLIYKDLVSV |
| 193 | ILQDGQFLV |
| 194 | SLLDYEVSI |
| 195 | LLGDSSFFL |
| 196 | VIFEGEPMYL |
| 197 | ALSYILPYL |
| 198 | FLFVDPELV |
| 199 | SEWGSPHAAVP |
| 200 | ALSELERVL |
| 201 | SLFESLEYL |
| 202 | KVLEYVIKV |
| 203 | VLLNEILEQV |
| 204 | SLLNQPKAV |
| 205 | KMSELQTYV |
| 206 | ALLEQTGDMSL |
| 207 | VIIKGLEEITV |
| 208 | KQFEGTVEI |
| 209 | KLQEEIPVL |
| 210 | GLAEFQENV |
| 211 | NVAEIVIHI |
| 212 | ALAGIVTNV |
| 213 | NLLIDDKGTIKL |
| 214 | VLMQDSRLYL |
| 215 | KVLEHVVRV |
| 216 | LLWGNLPEI |
| 217 | SLMEKNQSL |
| 218 | KLLAVIHEL |
| 219 | ALGDKFLLRV |
| 220 | FLMKNSDLYGA |
| 221 | KLIDHQGLYL |
| 222 | GPGIFPPPPPQP |
| 223 | ALNESLVEC |
| 224 | GLAALAVHL |
| 225 | LLLEAVWHL |
| 226 | SIIEYLPTL |
| 227 | TLHDQVHLL |
| 228 | SLLMWITQC |
| 229 | FLLDKPQDLSI |
| 230 | YLLDMPLWYL |
| 231 | GLLDCPIFL |
| 232 | VLIEYNFSI |
| 233 | TLYNPERTITV |
| 234 | AVPPPPSSV |
| 235 | KLQEELNKV |
| 236 | KLMDPGSLPPL |
| 237 | ALIVSLPYL |
| 238 | FLLDGSANV |
| 239 | ALDPSGNQLI |
| 240 | ILIKHLVKV |
| 241 | VLLDTILQL |
| 242 | HLIAEIHTA |
| 243 | SMNGGVFAV |
| 244 | MLAEKLLQA |
| 245 | YMLDIFHEV |
| 246 | ALWLPTDSATV |
| 247 | GLASRILDA |
| 248 | ALSVLRLAL |
| 249 | SYVKVLHHL |
| 250 | VYLPKIPSW |
| 251 | NYEDHFPLL |
| 252 | VYIAELEKI |
| 253 | VHFEDTGKTLLF |
| 254 | VLSPFILTL |
| 255 | HLLEGSVGV |

### Example 2

### Effect of Histone Deacetylase Inhibitors (HDACi) on T Cell Products

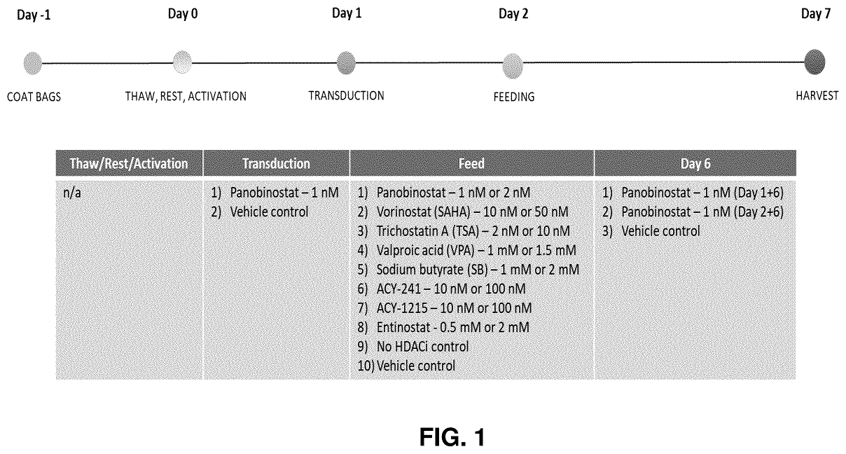

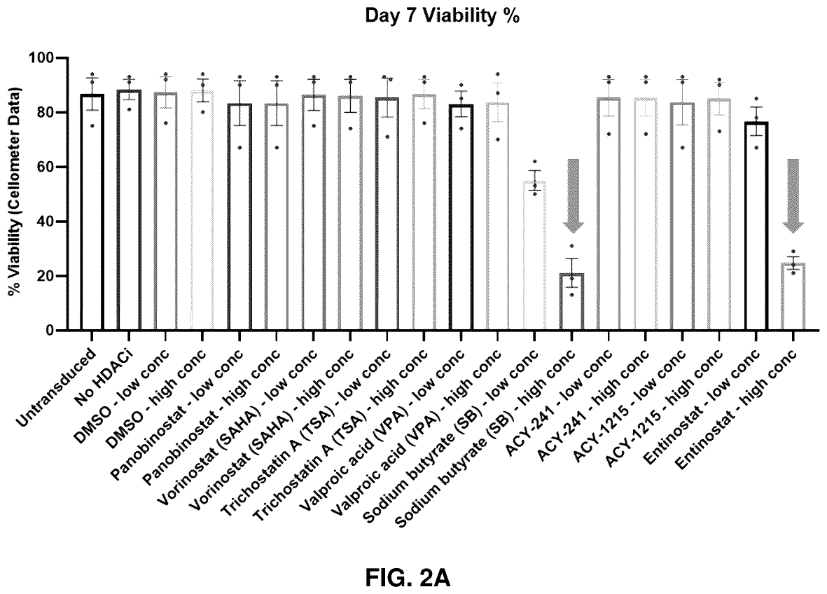

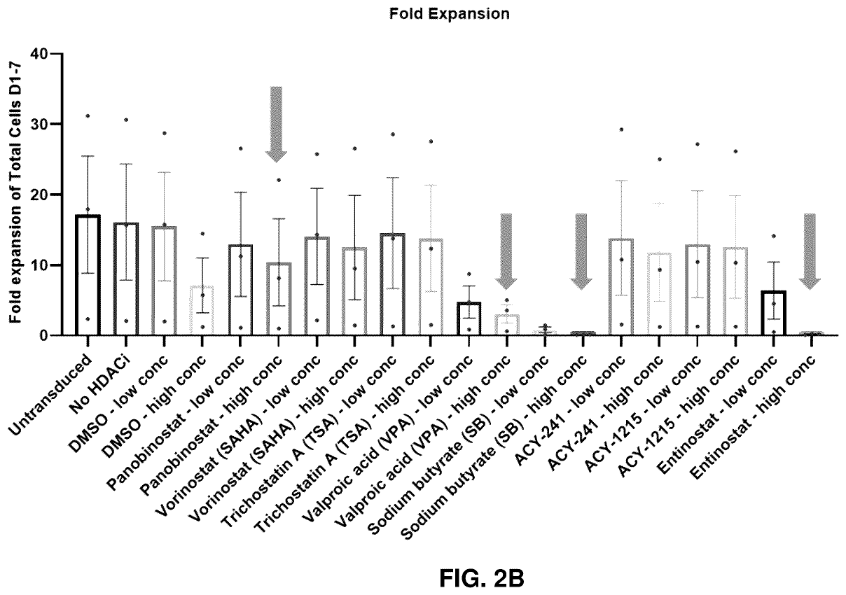

FIG. 1 shows an experimental design to test the effect of different HDACi on the T cell products. FIG. 2A shows viability of transduced T cells treated with different HDACi on Day 7. Sodium butyrate (SB) (2 mM) and Entinostat (2 mM), indicated by arrows, reduced T cell viability, as compared with the controls, e.g., no HDACi and DMSO. FIG. 2B shows Panobinostat (2 nM), valproic acid (VPA) (1.5 mM), sodium butyrate (2 mM), and Entinostat (2 mM) reduced fold expansion. (DMSO low concentration=low volume=0.00001%; vehicle control for Panobinostat at low concentration (1 nM); DMSO high concentration=high volume=1.5%; vehicle control for valproic acid at high concentration (1.5 mM); all except Na butyrate (water) were dissolved in DMSO)

### Effect of HDACi on Transduction Efficiency

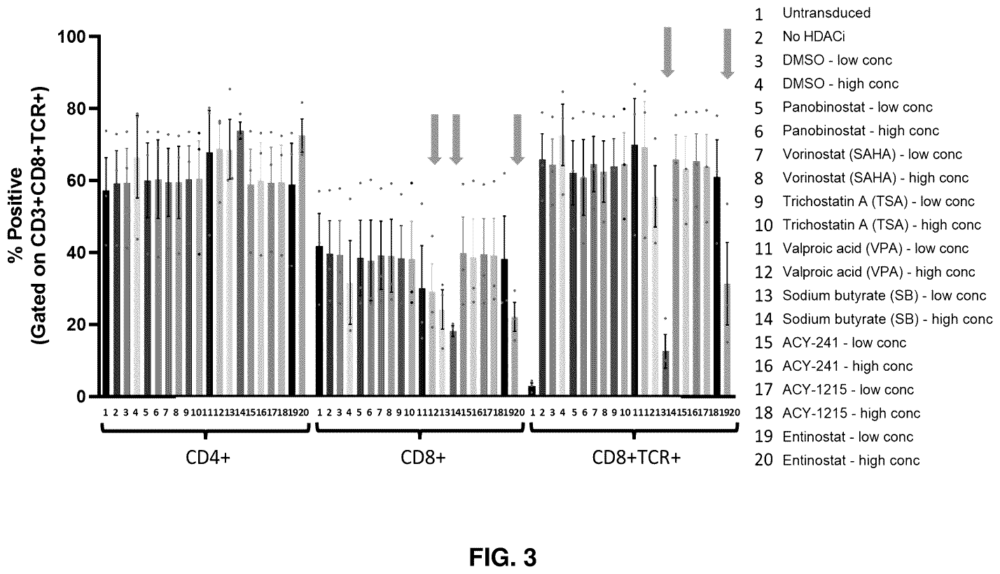

Transduction efficiency of transduced T cells treated with different HDACi were determined by flow cytometry gated on CD3+CD8+ TCR+ cells. FIG. 3 shows that valproic acid (1.5 mM), sodium butyrate (2 mM), and Entinostat (2 mM) reduced % CD8+ T cells. sodium butyrate (2 mM) and Entinostat (2 mM) reduced % CD8+ TCR+ cells, i.e., reduced transduction efficiency. HDACi may have little effect on % CD4+ T cells.

### Effect of HDACi on Memory T Cell Phenotype

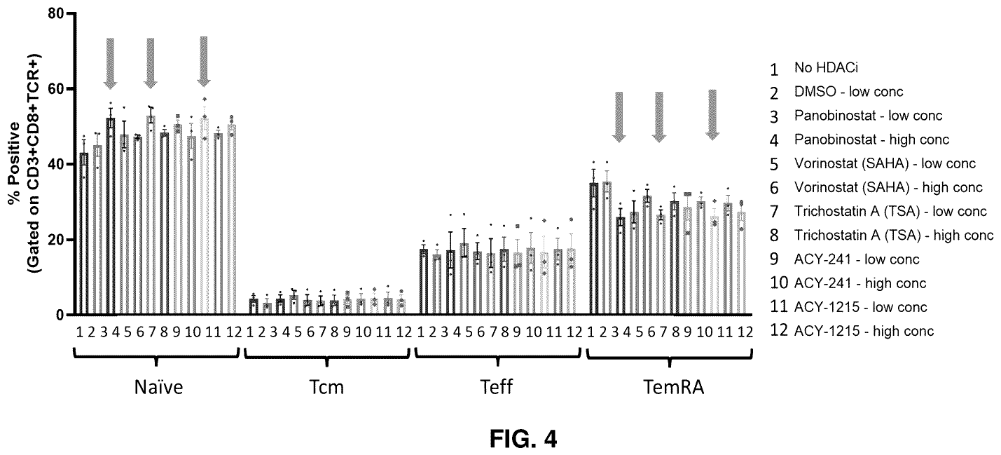

Phenotypes of transduced T cells treated with different HDACi were determined by flow cytometry gated on CD3+CD8+ TCR+ cells. FIG. 4 shows transduced T cells treated with Panobinostat (1 nM), SAHA (50 nM), or ACY-241 (100 nM), indicated by arrows, yielded most % Tnaïve cells and least TemRA cells, as compared with the other treatments and control (no HDACi). In addition, Panobinostat (1 nM), SAHA (50 nM), ACY-241 (100 nM) yielded more CD28+CD62L+ cells. No major difference in immune-checkpoint-inhibitor (ICI) marker expression was observed. These results suggest that Panobinostat (1 nM), SAHA (50 nM), or ACY-241 (100 nM) can promote Tnaive phenotype.

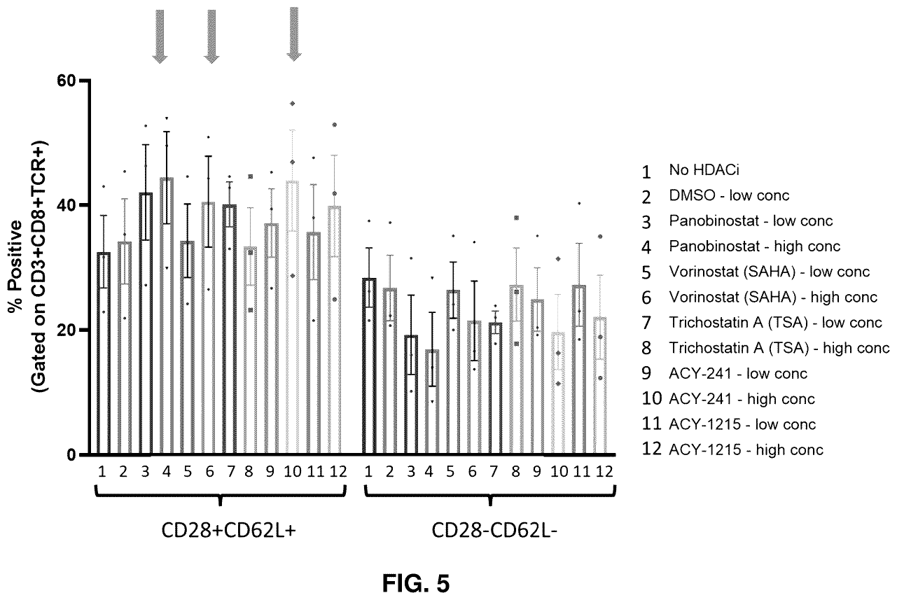

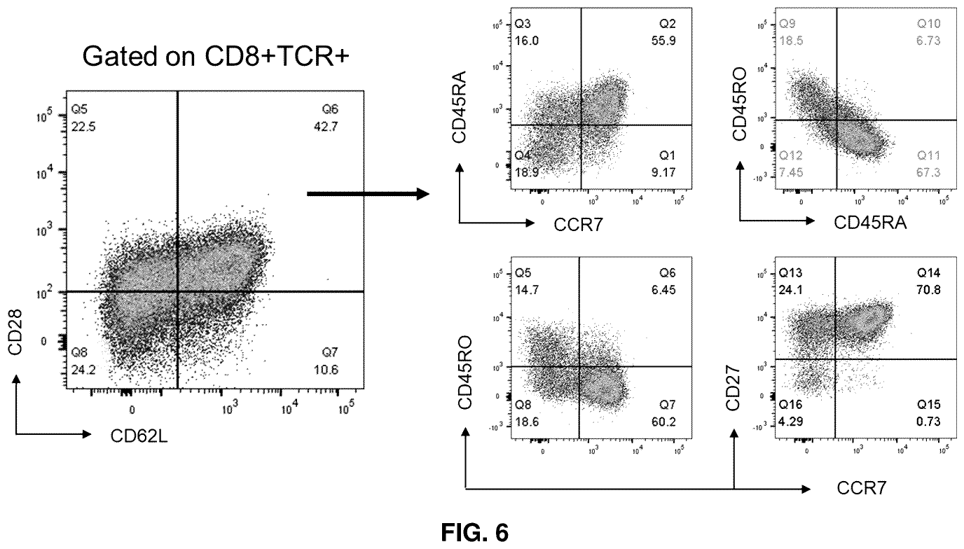

CD28+CD62L+ may be markers for Tcm-like cells. FIG. 5 shows that % CD28+CD62L+ cells (Tcm-like cells) increased in transduced T cells treated with Panobinostat (1 nM or 2 nM), SAHA (50 nM), or ACY-241 (100 nM), as indicated by arrows. FIG. 6 shows, however, CD28+CD62L+ cells exhibited 55.9% with Tnaive phenotypes, e.g., CD45RA+CCR7+, suggesting that CD28+CD62L+ may be not ideal markers for Tcm cells (CD45RA−CCR7+).

### Effect of HDACi Treatment at Different Times on T Cell Products

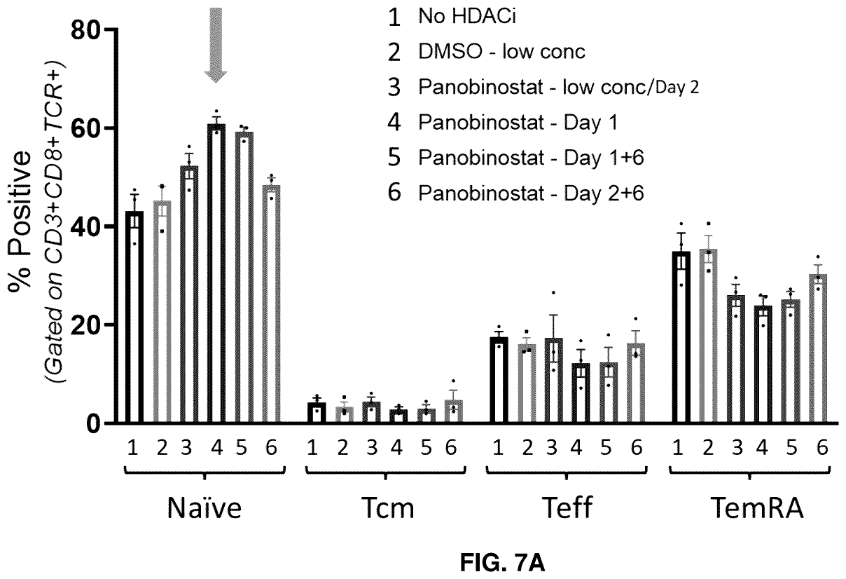

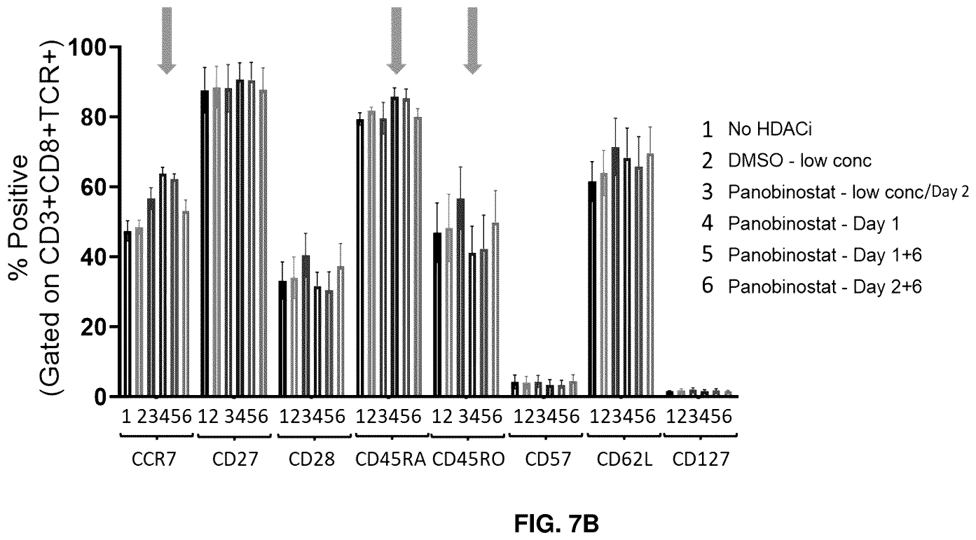

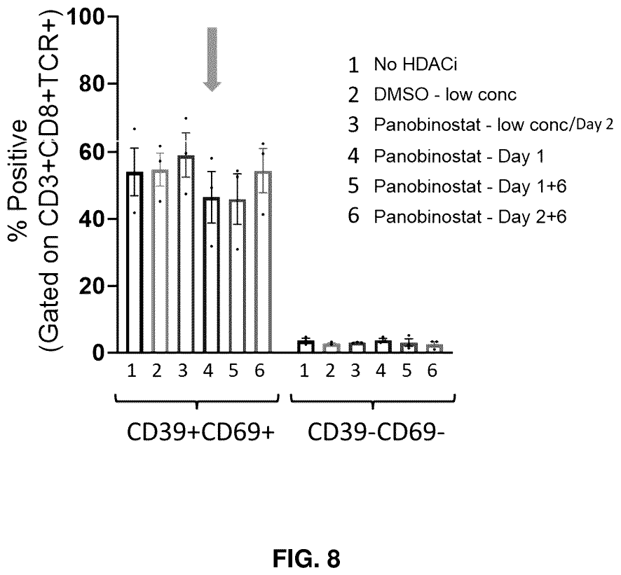

Using Panobinostat as an example, T cell products produced by treating T cells (i) on Day 1 (transduction) with Panobinostat (1 nM), (ii) on Day 2 with Panobinostat (1 nM), (iii) on Day 1 (transduction) with Panobinostat (1 nM) and Day 6 with Panobinostat (1 nM), and (iv) on Day 2 with Panobinostat (1 nM) and Day 6 with Panobinostat (1 nM) were compared. FIG. 7A shows the most % Tnaive and the least TemRA in T cell products prepared by Panobinostat (1 nM) treatment on Day 1 (transduction) as compared with the other treatments and control. Consistently, FIG. 7B shows T cell products prepared by Panobinostat (1 nM) treatment on Day 1 (transduction) exhibited higher % CCR7+ and CD45RA+ cells and lower % CD45RO+ cells as compared with controls (no HDACi and DMSO). FIG. 8 shows that T cell products prepared by Panobinostat (1 nM) treatment on Day 1 (transduction) exhibited lower % CD39+CD69+ cells, indicating fewer exhausted cells, as compared with controls (no HDACi and DMSO).

In sum, HDACi treatment can lead to a more favorable memory T cell phenotype. HDACi treatment increased naïve cells, e.g., −25% increase over control, with fewer TemRA cells and very few Tcm cells in engineered T cell manufacturing process. No major difference in phenotypes were observed with different HDACi treatments. In addition, sodium butyrate, entinostat, and valproic acid appear to be highly toxic. Panobinostat (1 nM), SAHA (50 nM), and ACY-241 (100 nM) performed best, e.g., resulting in most naïve cells with similar ICI expression as compared with the other treatments and control. Panobinostat (1 nM) appears less toxic than Panobinostat (2 nM), suggesting the potentially negative impact of HDACi on fold expansion may be avoided by lowering the dose of HDACi, e.g., Panobinostat. The time to treat with HDACi may be on Day 1 (transduction), Day 2, or Day 6. The time to treat with HDACi may be on Day 1 or on Day 2. The time to treat with HDACi may be on Day 1.

### Example 3

### Effect of HDACi+IL-21 Treatment at Different Times on T Cell Products

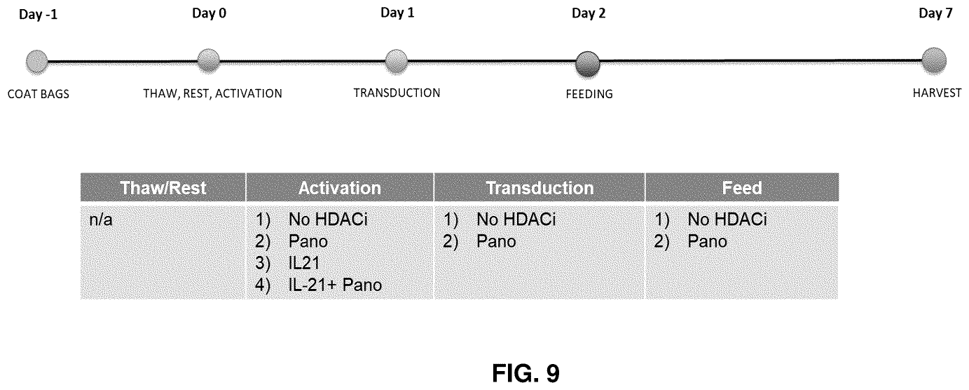

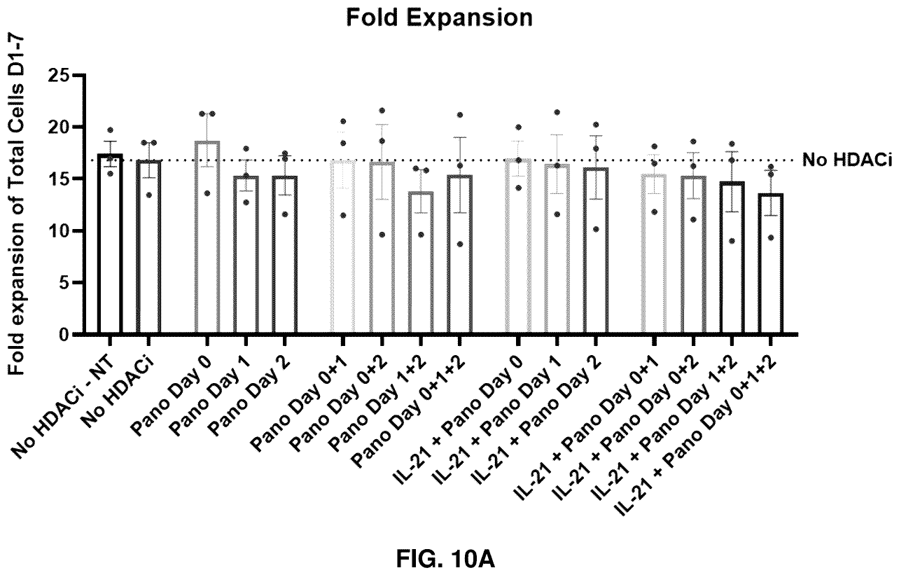

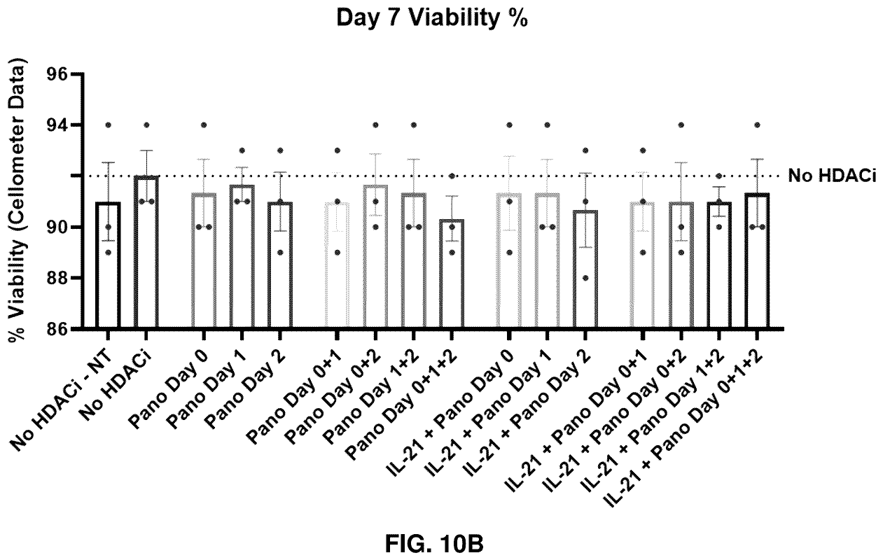

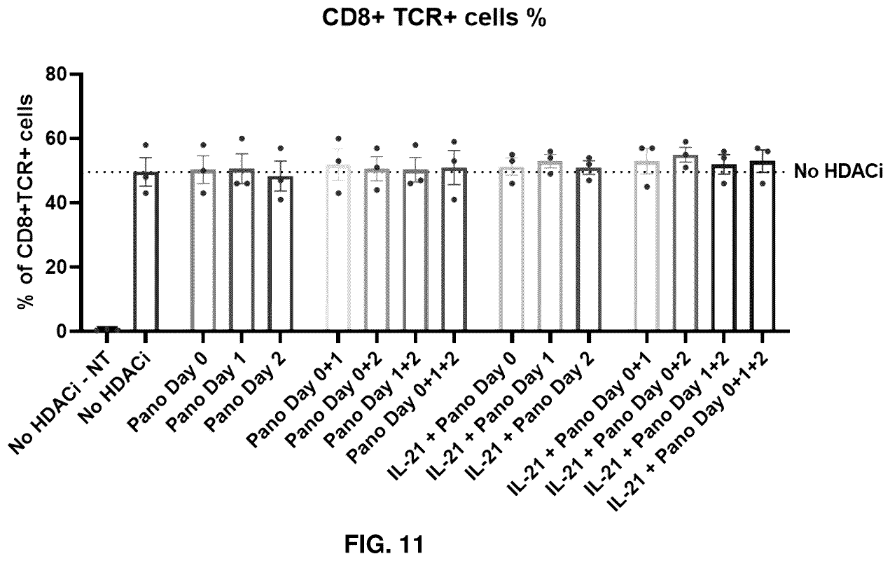

FIG. 9 shows an experimental design to test the effect of treatment of HDACi (Panobinostat (1 nM) or SAHA (10 nM))+IL-21 (30 ng) at activation (Day 0), transduction (Day 1), and/or feeding (Day 2) on the T cell products. FIG. 10A shows addition of Panobinostat (without IL-21) on Day 0 led to an increase in fold expansion. Addition of Panobinostat+IL-21 had little effect on fold expansion as compared with the control (no HDACi). FIG. 10B shows HDACi with or without IL-21 decreased viability as compared with the control (no HDACi). FIG. 11 shows HDACi with or without IL-21 had little effect on transduction efficiency as compared with the control (no HDACi).

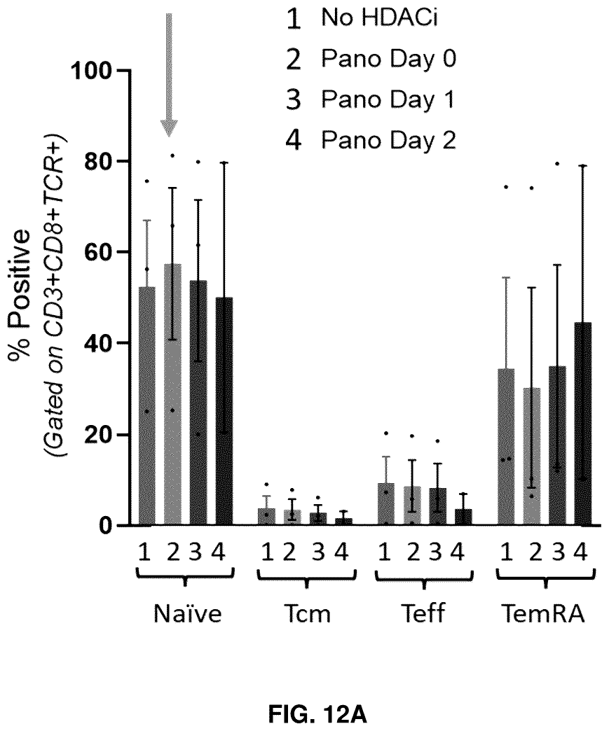

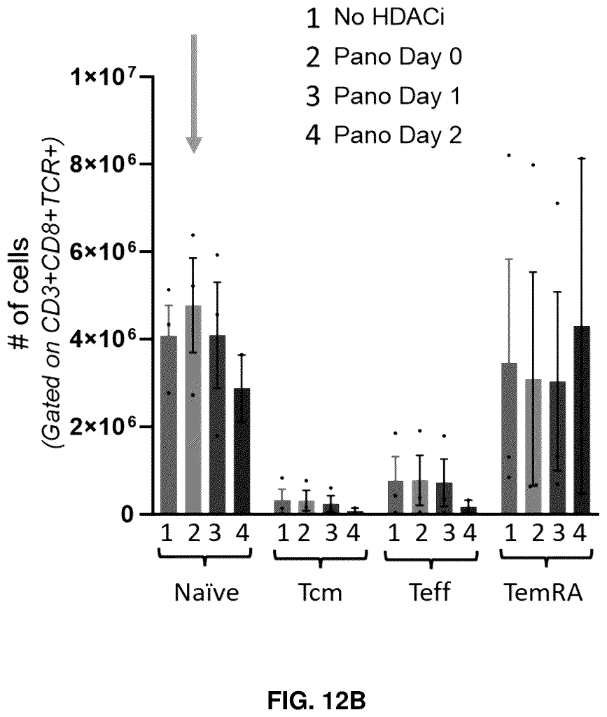

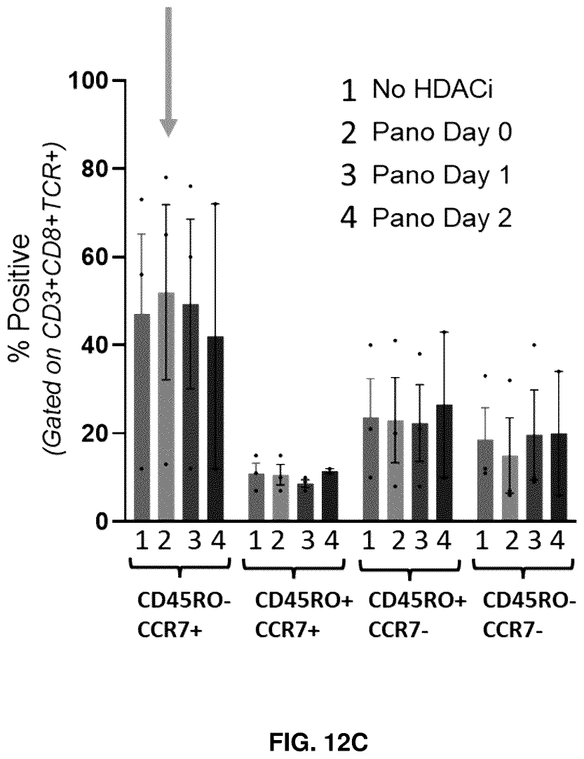

Using Panobinostat as example, phenotypes of T cell products prepared by Panobinostat treatment on Day 0 (activation), Day 1 (transduction), or Day 2 (feeding) were determined. FIG. 12A shows T cell products prepared by Panobinostat treatment on Day 0 (activation) yielded most % Tnaive cells and least TemRA cells as compared with the other treatments and control. FIG. 12B shows T cell products prepared by Panobinostat treatment on Day 0 (activation) yielded highest number of Tnaive cells as compared with the other treatments and control. Consistently, FIG. 12C shows T cell products prepared by Panobinostat treatment on Day 0 (activation) yielded highest % CD45RO−CCR7+ T cells as compared with the other treatments and control.

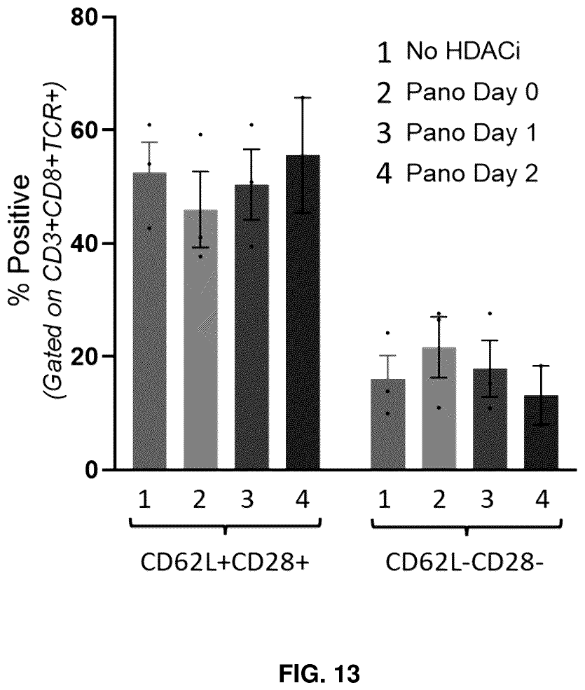

FIG. 13 shows that T cell products prepared by Panobinostat treatment on Day 0 (activation) yielded lower % CD62L+CD28+ T cells as compared with control (no HDACi).

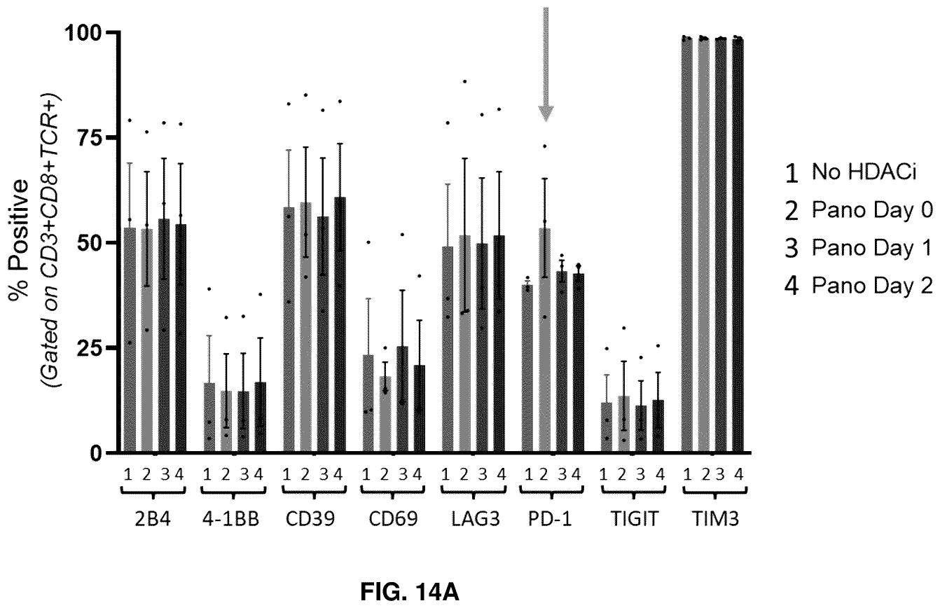

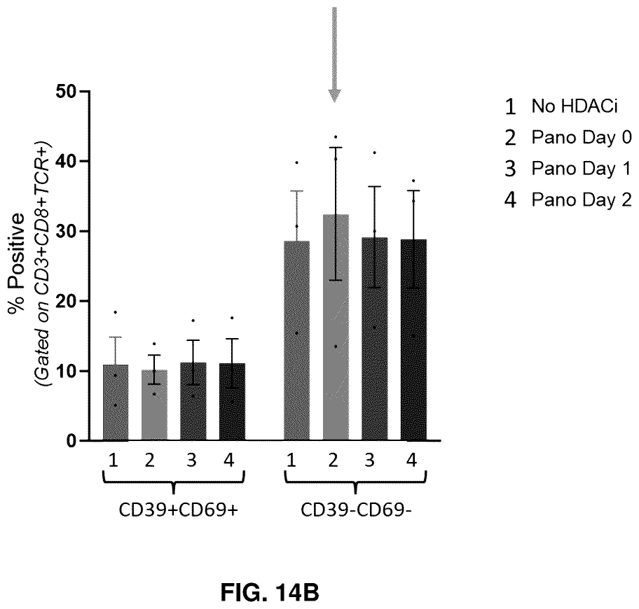

The sustained expression of multiple inhibitory receptors may be the hallmark of exhausted T cells. Common to both CD4+ and CD8+ exhausted T cells may be the surface expression of multiple inhibitory receptors, e.g., 2B4, 4-1BB, CD39, CD69, LAG3, PD-1, TIGIT, and TIM3. Expression of these inhibitory receptors in T cell products prepared by Panobinostat treatment at different time was determined. FIG. 14A shows ICI markers mostly unchanged across conditions except PD-1, indicated by an arrow. Panobinostat treatment on Day 0 increased % PD-1+ cells in T cell products as compared with other treatments and control. FIG. 14B shows Panobinostat treatment on Day 0 tends to increase % T cells with less exhausted phenotypes, e.g., CD39-CD69-, indicated by an arrow.

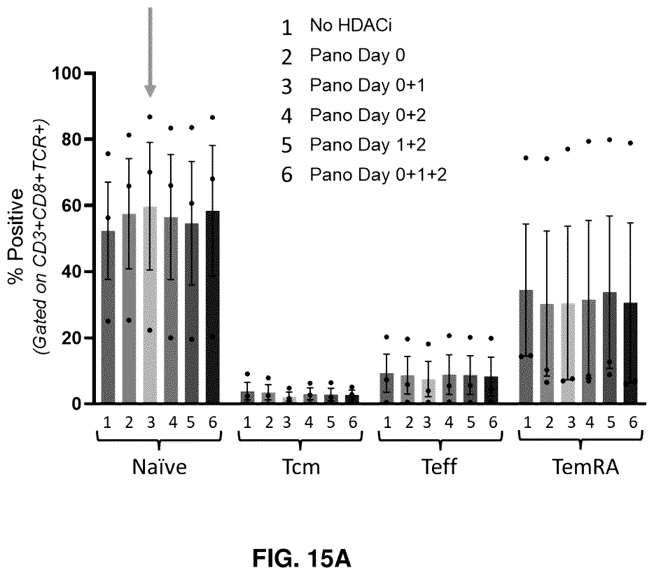

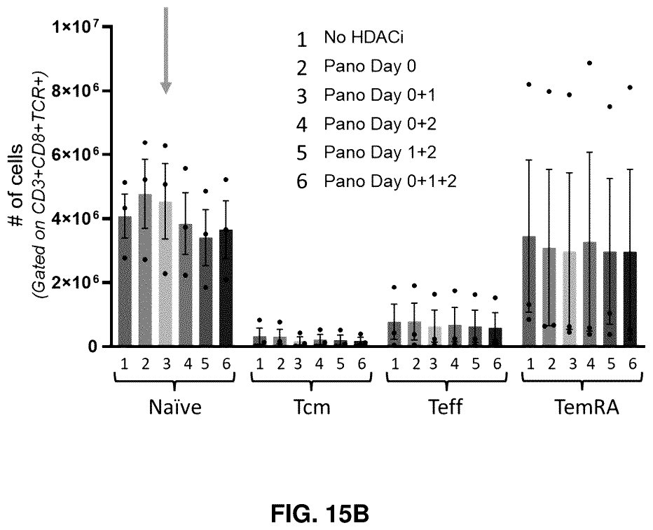

To test the effect of HDACi treatment frequency on T cell products, phenotypes of T cell products prepared by Panobinostat treatment at Day 0 (activation), Day 0 (activation)+Day 1 (transduction), Day 0 (activation)+Day 2 (feeding), Day 1 (transduction)+Day 2 (feeding), and Day 0 (activation)+Day 1 (transduction)+Day 2 (feeding), were determined. FIG. 15A shows Panobinostat treatment twice at Day 0 and Day 1 yielded most % T cell products with Tnaive phenotypes, indicated by an arrow, as compared with other treatments and control. FIG. 15B shows, due to slightly better fold expansion with Panobinostat treatment at day 0 as compared with Panobinostat treatment at day 0+1, more number of Tnaive cells were obtained from the former than that obtained from the latter.

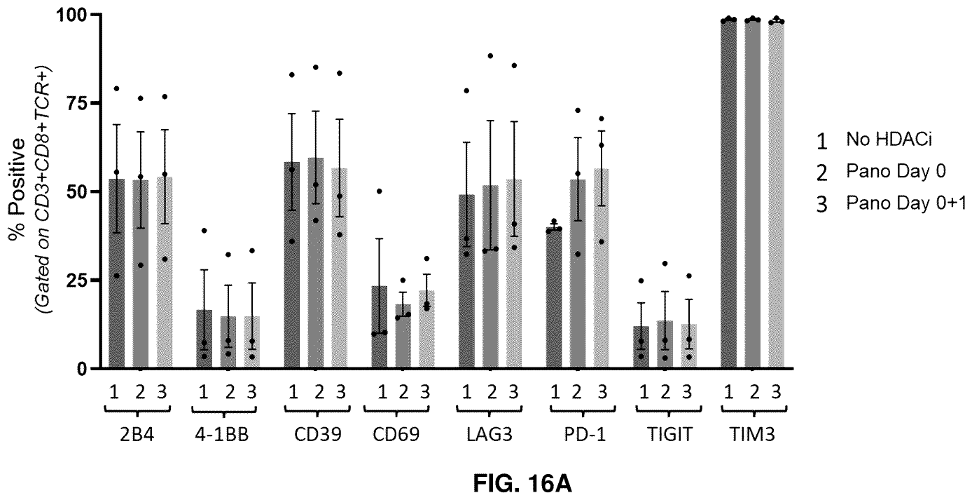

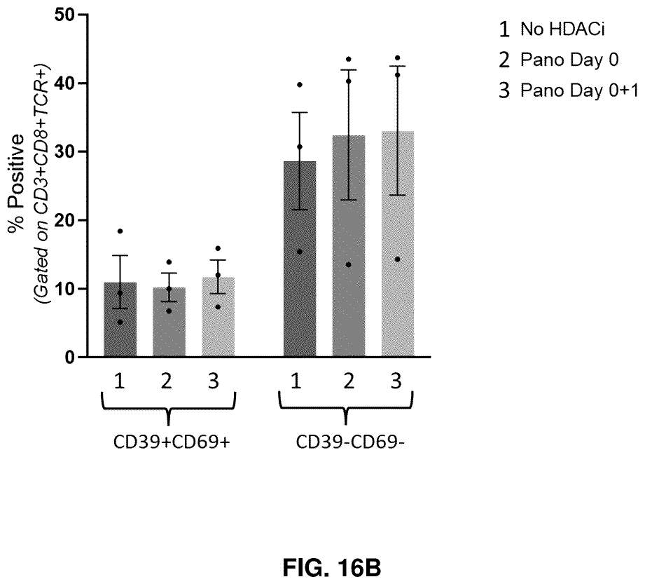

T cell products prepared by Panobinostat treatment at Day 0 or Day 0+Day 1 were further examined for the expression of inhibitory receptors. FIG. 16A shows no significant difference in inhibitory receptors in T cell products prepared by Panobinostat treatment at Day 0 and at Day 0+Day 1. FIG. 16B shows Panobinostat treatment at Day 0 and at Day 0+Day 1 had little effect on % T cells with exhausted phenotypes, e.g., CD39+CD69+, or with less exhausted phenotypes, e.g., CD39−CD69−.

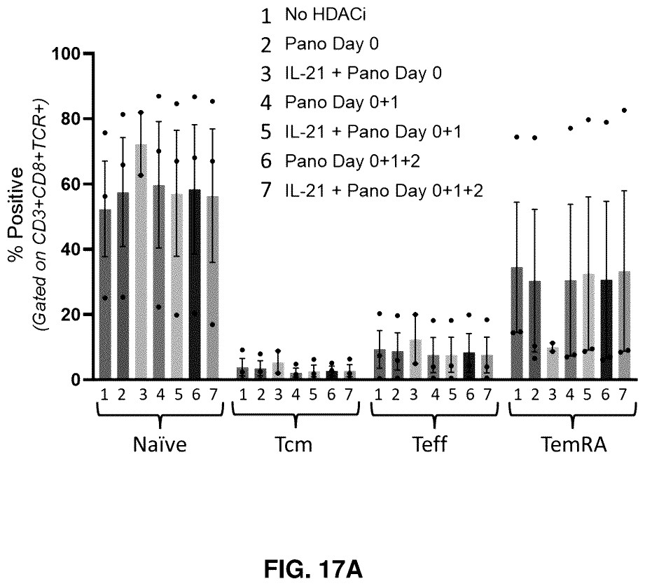

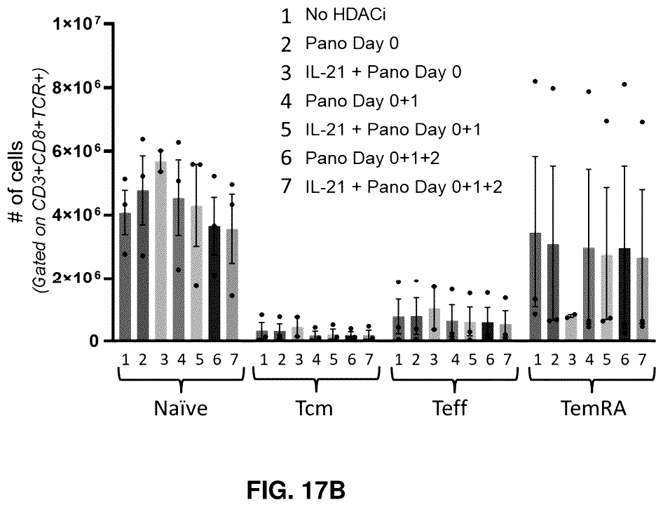

To test whether IL-21 can improve T cell phenotype, characteristics of T cell products prepared by Panobinostat treatment with or without IL-21 were determined. FIGS. 17A and 17B show addition of IL-21 may not have a significant effect on % and # of Tnaive cells as compared with other treatments and control.

### Example 4

TABLE 4

| | Panobinostat | SAHA | AKTi VIII |
|---|---|---|---|
| Type | HDACI | HDACi | AKTi |
| Concentration | 0.5 nM | 0.5 nM | 250 nM |
| Timepoint | Day 0 | Day 0 | Day 0 |
| Combination with IL-21 | No | Yes | No |

### Effect of HDACi and AKT Inhibitors (AKTi) on T Cell Products

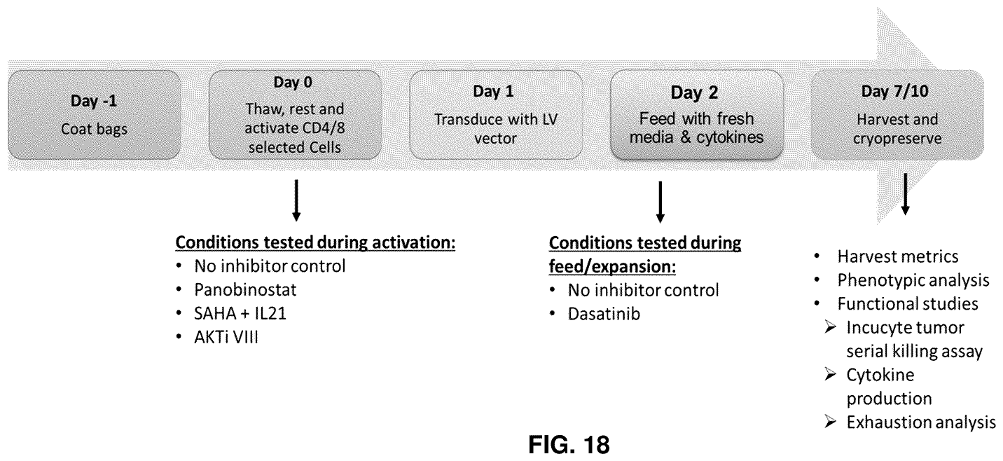

FIG. 18 and Table 4 show that, to test the effects of HDACi and AKTi on T cell products, on Day 0, panobinostat (Pano) (0.5 nM), or SAHA (0.5 nM)+IL-21 (30 ng/ml), or AKTi VIII (250 nM) was added during activation of CD4/CD8-selected cells in the presence of anti-CD3/anti-CD28 antibodies. On Day 1, the activated CD4/CD8-selected cells were transduced with Lentiviral vectors expressing a target-specific exogenous TCR. On Day 2, the culture media were removed and the transduced cells were fed with fresh media in the absence of Panobinostat, SAHA+IL-21, and AKTi VIII and in the presence of cytokines, for cell expansion. On Day 7-Day 10, cells were harvested and cryopreserved for harvest metrics, phenotypic analysis, and functional studies including Incucyte tumor serial killing assay, cytokine production, and exhaustion analysis. To test the effects of TKi, e.g., dasatinib, on T cell products, dasatinib was added on Day 2 (5 nM) and Day 5 (10 nM) during feeding and expansion.

AKTi-treated cells have relatively better manufacturing metrics and phenotype as compared with Panobinostat and SAHA+IL-21 treated cells.

T cell products prepared by activating T cells obtained from three patients (n=3) in the presence of Panobinostat, SAHA+IL-21, or AKTi VIII were compared with regards to manufacturing metrics and phenotype.

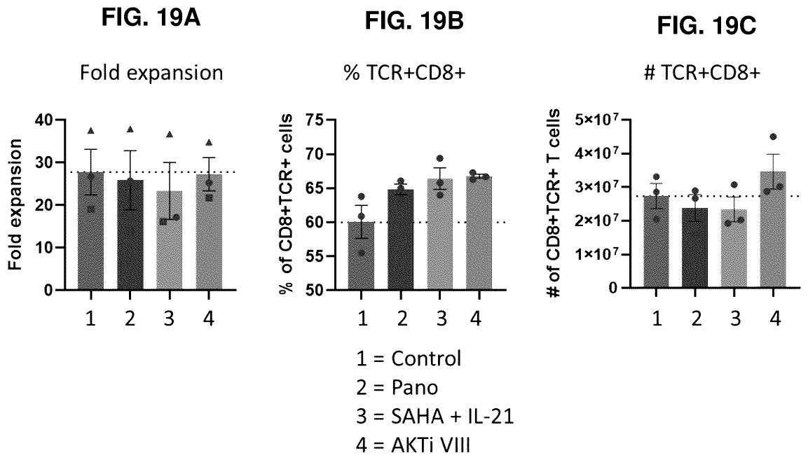

FIG. 19A shows activation in the presence of Panobinostat or AKTi VIII may have little effect on the fold expansion of T cell products as compared with that of the untreated control. Activation in the presence of SAHA+IL-21 slightly decreased the fold expansion of T cell products as compared with that of the untreated control. FIG. 19B shows that activation in the presence of Panobinostat, SAHA+IL-21, or AKTi VIII yielded T cell products with higher % CD8+ TCR+ T cells than that of the untreated control, suggesting that activation in the presence of Panobinostat, SAHA+IL-21, or AKTi VIII can improve transduction efficiency. FIG. 19C shows that activation in the presence of AKTi VIII yielded the best total number of T cell products with CD8+ TCR+ T cells as compared with that treated with Panobinostat, or SAHA+IL-21, or the untreated control.

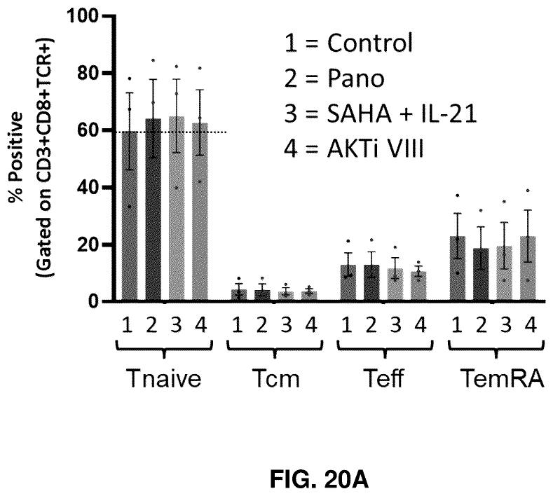

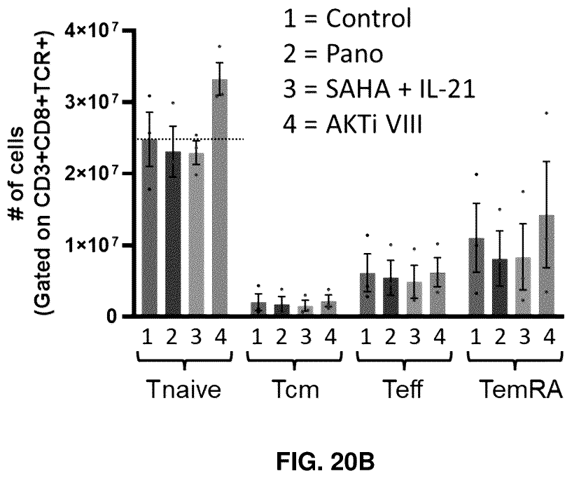

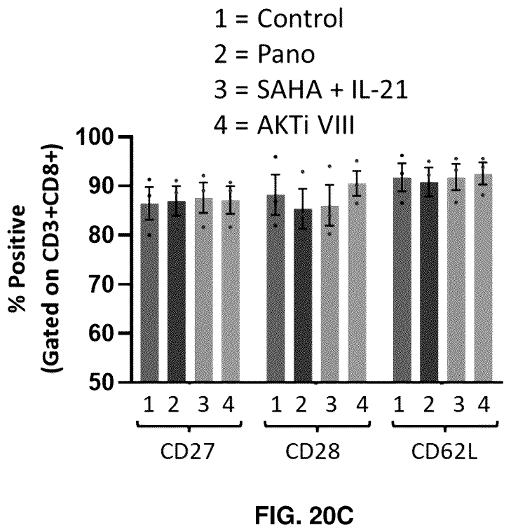

FIG. 20A shows activation in the presence of Panobinostat, SAHA+IL-21, or AKTi VIII increased % Tnaive (CD45RA+CCR7+) in T cell products as compared with that of the untreated control. It appears that Panobinostat, SAHA+IL-21, or AKTi VIII may have little effect on the % of Tcm (CD45RA−CCR7+), Teff (CD45−CCD7−), and TemRA (CD45RA+CCR7-) in the T cell products. FIG. 20B shows activation in the presence of AKTi VIII yielded the best total number of Tnaive (CD45RA+CCR7+) in T cell products as compared with that treated with Panobinostat or SAHA+IL-21 and the untreated control. FIG. 20C shows that activation in the presence of Panobinostat, SAHA+IL-21, or AKTi VIII may have little effect on the expression of Tnaive and Tcm markers, e.g., CD27, CD28, and CD62L, in T cell products as compared with that of the untreated control.

AKTi-treated cells exhibit better tumor-killing activity than untreated cells.

T cell products prepared by activating T cells obtained from three patients (n=3) in the presence of Panobinostat, SAHA+IL-21, or AKTi VIII were compared with regards to tumor killing activity when contacting target cells, e.g., UACC257, which express a report gene encoding red florescent protein (RFP), with E:T=3:1. TCR-positive cells were normalized.

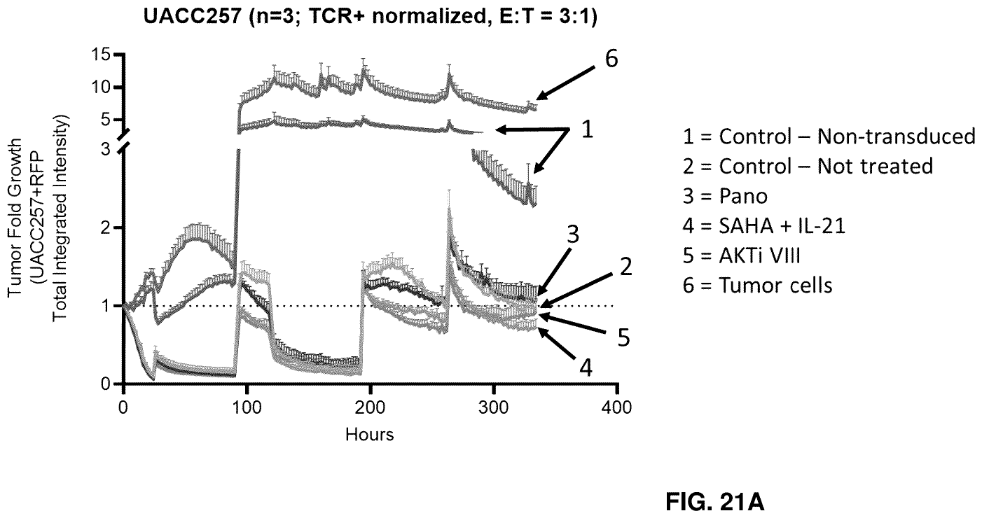

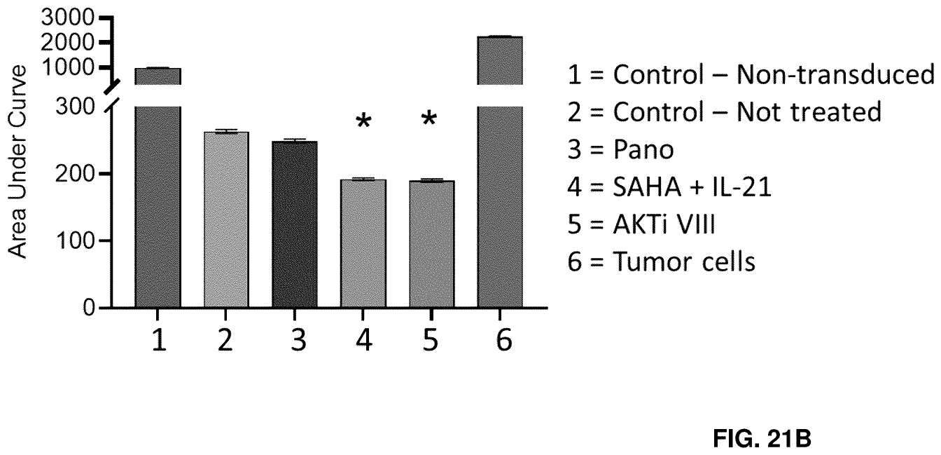

FIG. 21A shows that T cell products prepared by activating T cells in the presence of SAHA+IL-21 or AKTi VIII exhibited better tumor killing activity as compared with that treated with Panobinostat and the controls, e.g., non-transduced, non-treated, and tumor cells. FIG. 21B shows the quantitative results of that shown in FIG. 6A. *p<0.05, **p<0.01, ***p<0.001 by one-way ANOVA with Tukey's test for multiple comparisons; compared to Control condition. Similar results were observed using T cells obtained from healthy donors. (Data not shown).

### Example 5

### Effect of Tyrosine Kinase Inhibitors (TKi) on T Cell Products

Dasatinib-Treated Cells have a Better Phenotype than Untreated Cells

Dasatinib (CAS No. 302962-49-8) was purchased from AdipoGen Life Sciences. (adipogen.com/ag-cr1-3540-dasatinib.html/).

FIG. 18 shows that, to test the effects of TKi, e.g., dasatinib, on T cell products, dasatinib was added on Day 2 or on Day 5 during feeding and expansion. On Day 0, thawed and rested CD4/CD8-selected cells were activated in the presence of anti-CD3/anti-CD28 antibodies. On Day 1, the activated CD4/CD8-selected cells were transduced with Lentiviral vectors expressing a target-specific exogenous TCR and exogenous CD8 molecules. On Day 2, the culture media were removed and the transduced cells were fed with fresh media containing cytokines for cell expansion. Dasatinib was added on Day 2 or on Day 5. On Day 7-Day 10, cells were harvested and cryopreserved for harvest metrics, phenotypic analysis, and functional studies including Incucyte tumor serial killing assay, cytokine production, and exhaustion analysis.

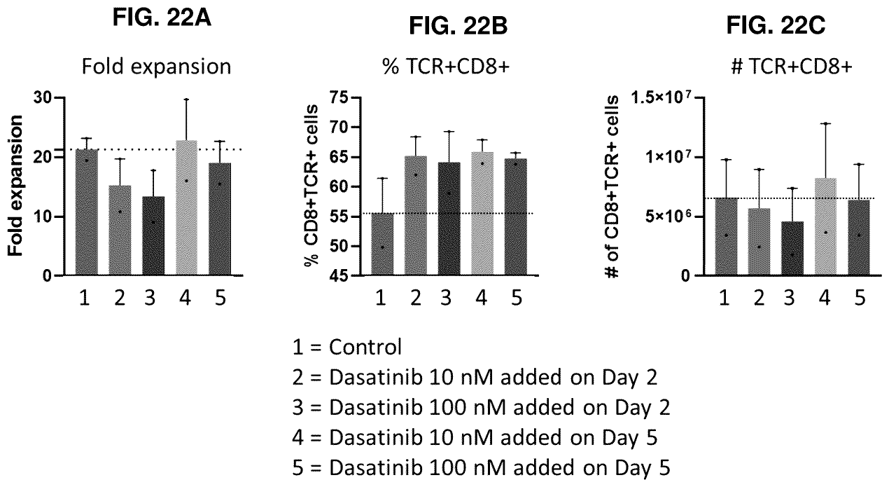

FIG. 22A shows expansion in the presence of dasatinib on Day 2 (10 nM or 100 nM) or Day 5 (10 nM or 100 nM) may have little effect on the fold expansion of T cell products as compared with that of the untreated control. FIG. 22B shows that expansion in the presence of dasatinib on Day 2 (10 nM or 100 nM) or Day 5 (10 nM or 100 nM) yielded T cell products with higher % CD8+ TCR+ T cells than that of the untreated control, suggesting that expansion in the presence of dasatinib can improve transduction efficiency. FIG. 22C shows that expansion in the presence of dasatinib may have little effect on total number of T cell products with CD8+ TCR+ T cells as compared with that of the untreated control.

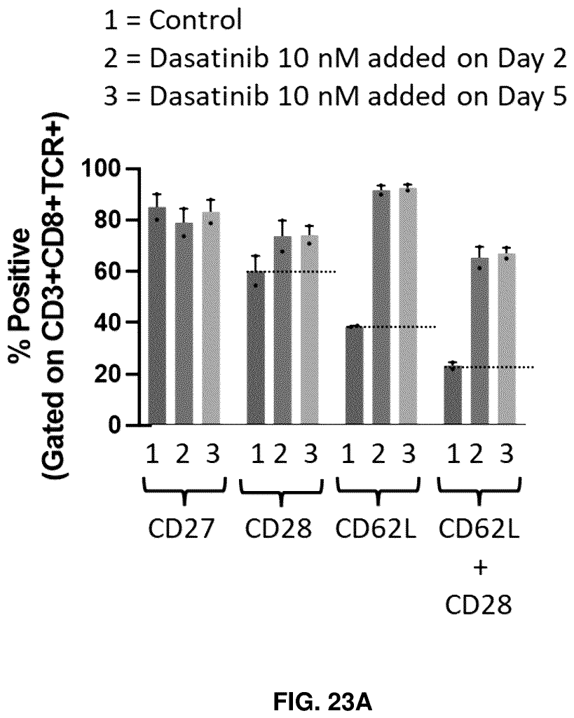

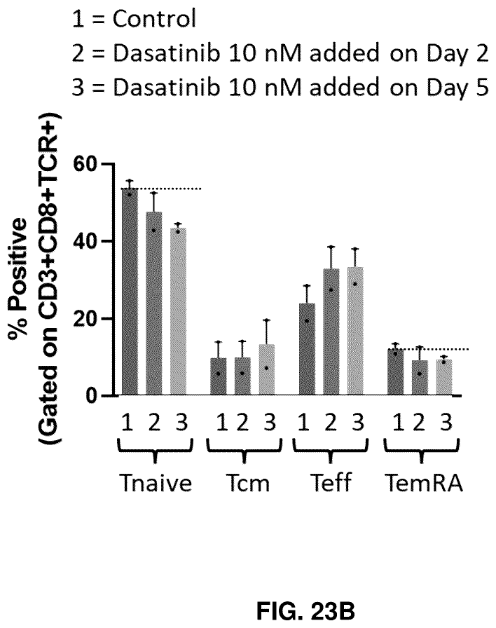

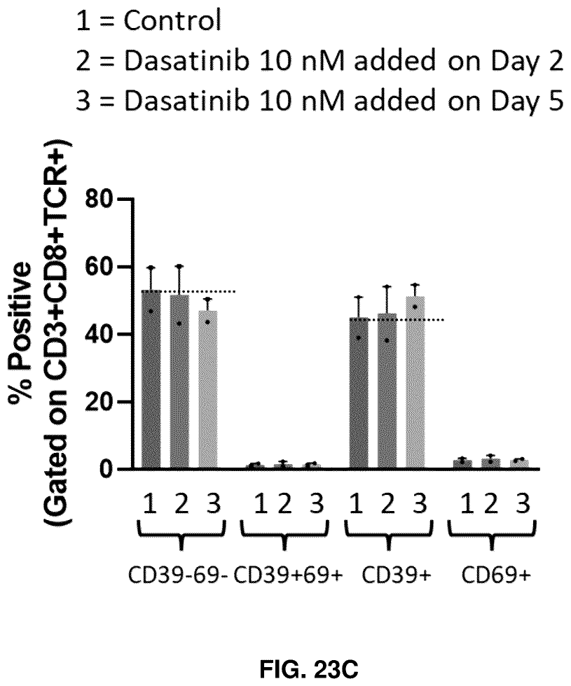

FIG. 23A shows expansion in the presence of dasatinib (10 nM) on Day 2 or Day 5 increased the expression of Tnaive and Tcm markers, e.g., CD28 and CD62L, in T cell products as compared with that of the untreated control. Little effect on the expression of CD27 was observed. FIG. 23B shows, while expansion in the presence of dasatinib (10 nM) on Day 2 or Day 5 yielded less % Tnaive in T cell products, less % of differentiated TemRA were obtained as well.

CD39 may be considered as a marker for exhausted T cells, e.g., CD8+ T cells, in cancer. CD69 is a membrane-bound, type II C-lectin receptor and may be considered as an early marker of lymphocyte activation due to its rapid appearance on the surface of the plasma membrane after stimulation. FIG. 23C shows that expansion in the presence of dasatinib (10 nM) on Day 2 or Day 5 yielded slightly less % of T cell products with memory-progenitor CD39− negative stem-like phenotype (CD39−CD69−) than that of the untreated control. On the other hand, expansion in the presence of dasatinib (10 nM) on Day 2 or Day 5 yielded slightly higher % of T cell products with differentiated CD39+ state (CD39+) than that of the untreated control. Dasatinib may have little effect on % of terminally differentiated CD39-positive state (CD39+CD69+) and % of CD69+ state in T cell products.

### Example 6

### Comparative Studies

Inhibitor-treated cells have mostly comparable to better fold expansion and yield of TCR+CD8+ T cells compared to untreated cells except SAHA+IL-21

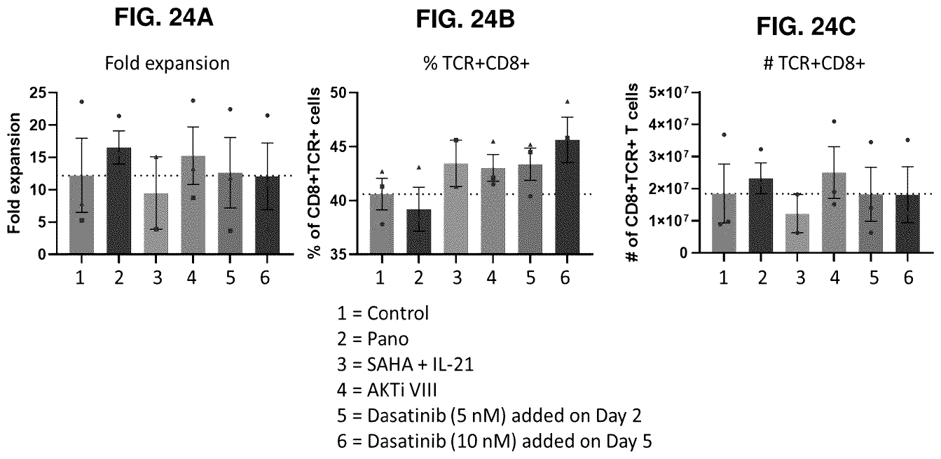

FIG. 24A shows activation in the presence of Panobinostat or AKTi VIII or expansion in the presence of dasatinib may have little effect on the fold expansion of T cell products as compared with that of the untreated control. Consistent with the fold expansion shown in FIG. 19A, activation in the presence of SAHA+IL-21 slightly decreased the fold expansion of T cell products as compared with that of the untreated control. FIG. 24B shows that activation in the presence of Panobinostat, SAHA+IL-21, or AKTi VIII or expansion in the presence of dasatinib yielded T cell products with higher % CD8+ TCR+ T cells than that of the untreated control, suggesting that activation in the presence of Panobinostat, SAHA+IL-21, or AKTi VIII or expansion in the presence of dasatinib can improve transduction efficiency. FIG. 24C shows that activation in the presence of Panobinostat, SAHA+IL-21, or AKTi VIII or expansion in the presence of dasatinib may have little effect on total number of T cell products with CD8+ TCR+ T cells as compared with that of the untreated control.

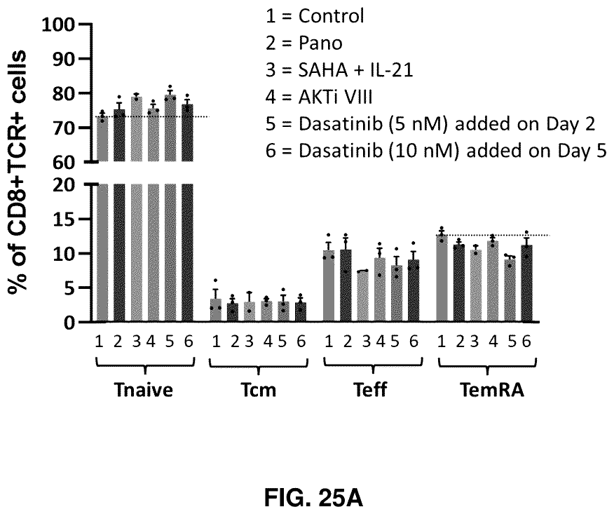

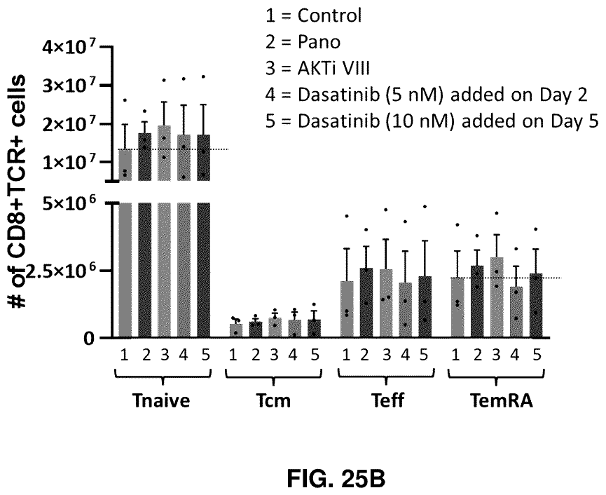

FIG. 25A shows activation in the presence of Panobinostat, SAHA+IL-21, or AKTi VIII or expansion in the presence of dasatinib increased % Tnaive in T cell products and decreased % TemRA as compared with that of the untreated control. It appears that adding dasatinib (5 nM) on Day 2 yielded more % Tnaive and less % TemRA in T cell products than adding dasatinib (10 nM) on Day 5. FIG. 25B shows that activation in the presence of Panobinostat or AKTi VIII or expansion in the presence of dasatinib increased number of Tnaive in T cell products as compared with that of the untreated control. Ranking of T cell products containing from the most Tnaive and the least TemRA to the least Tnaive and the most TemRA may be dasatinib (5 nM) on Day 2 >dasatinib (10 nM) on Day 5 >AKTi VIII >Panobinostat.

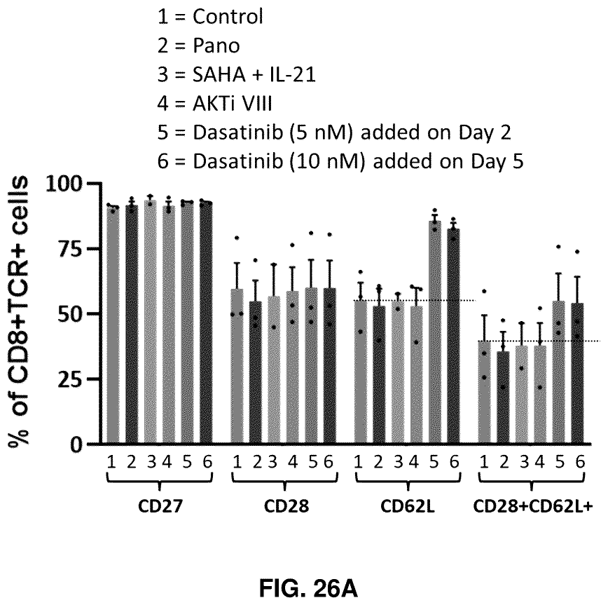

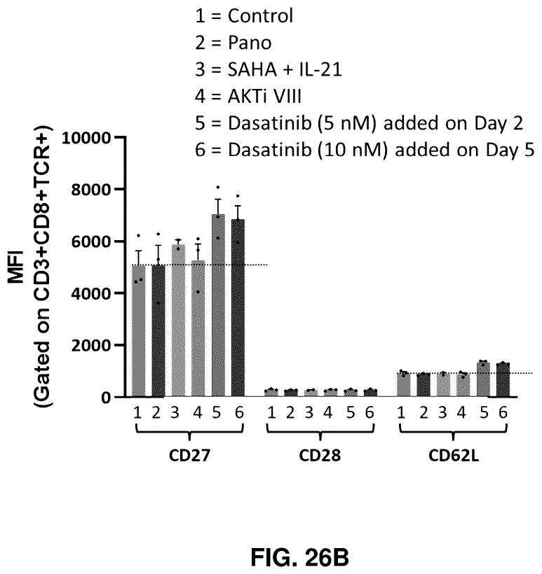

FIG. 26A shows expansion in the presence of dasatinib (10 nM) on Day 2 or Day 5 increased % CD62L+ and % CD28+CD62L+ cells in T cell products as compared with that treated with Panobinostat, SAHA+IL-21, or AKTi VIII, or the untreated control, suggesting that expansion in the presence of dasatinib may increase % Tnaive in T cell products. FIG. 26B shows expansion in the presence of dasatinib (10 nM) on Day 2 or Day 5 increased MFI of CD27 and CD62L in T cell products as compared with that treated with Panobinostat, SAHA+IL-21, or AKTi VIII, or the untreated control, suggesting that expansion in the presence of dasatinib may increase expression of CD27 and CD62L in T cell products.

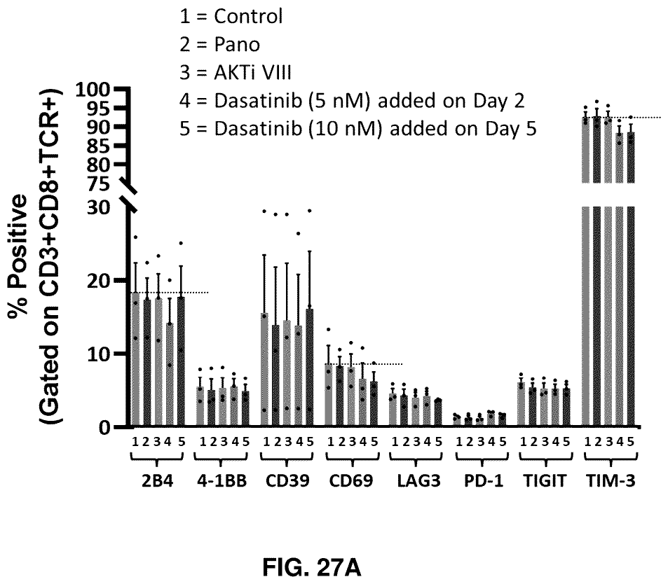

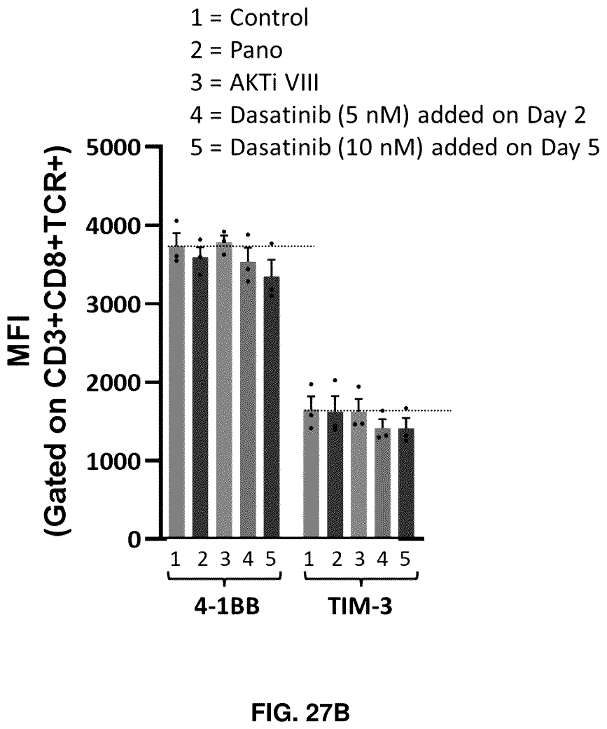

Common to both CD4+ and CD8+ exhausted T cells may be the surface expression of multiple inhibitory receptors including 2B4, 4-1BB, CD39, CD69, LAG3, PD-1, TIGIT, and TIM-3. FIG. 27A shows expansion in the presence of dasatinib (10 nM) on Day 2 or Day 5 decreased % 2B4+, % Cd69+, and % TIM-3+ cells in T cell products as compared with that treated with Panobinostat or AKTi VIII, or the untreated control, suggesting that expansion in the presence of dasatinib may reduce T cell exhaustion. FIG. 27B shows expansion in the presence of dasatinib (10 nM) on Day 2 or Day 5 decreased MFI of 4-1BB and TIM-3 in T cell products as compared with that treated with Panobinostat or AKTi VIII, or the untreated control, suggesting that expansion in the presence of dasatinib may decrease expression of 4-1BB and TIM-3 in T cell products.

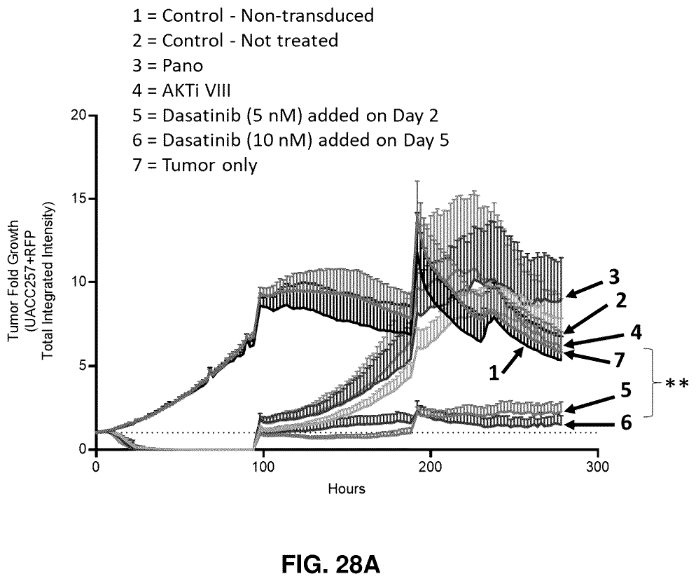

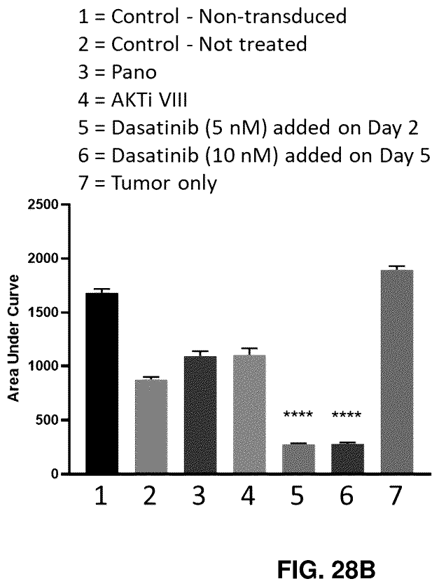

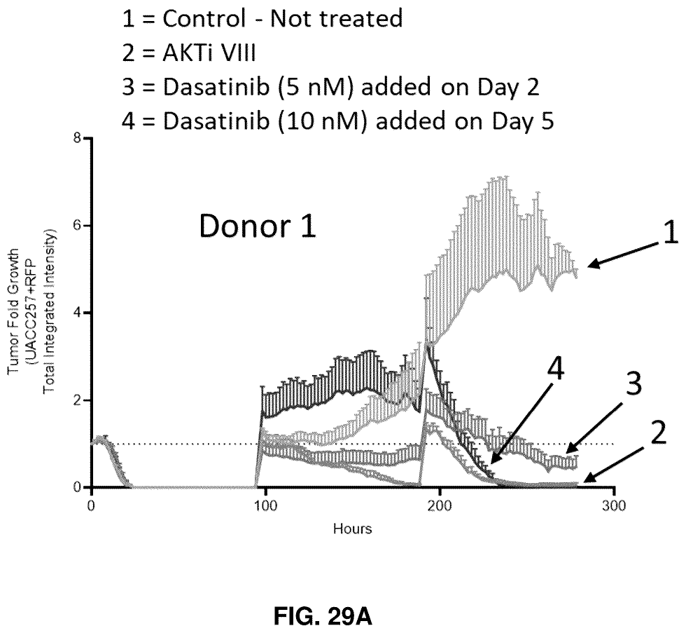

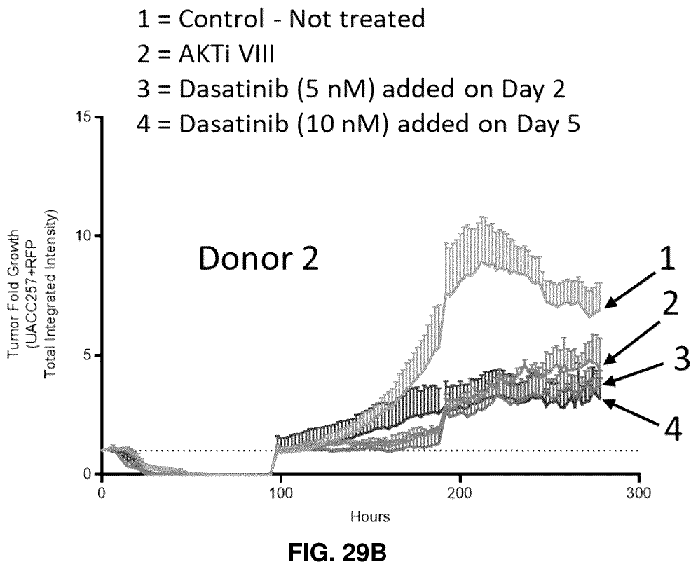

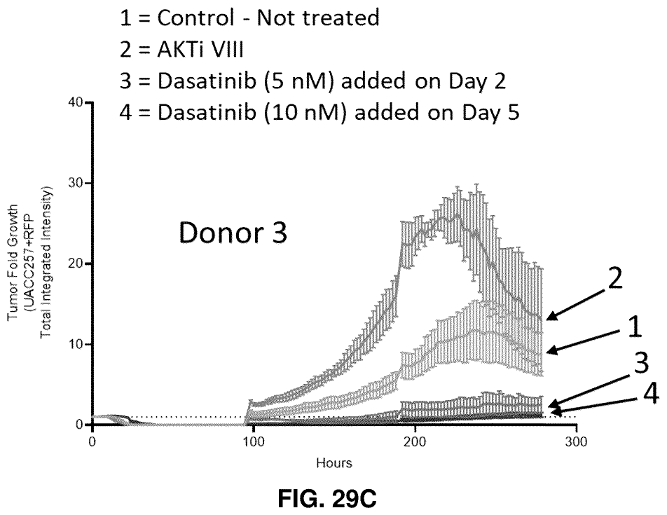

FIG. 28A shows that T cell products prepared by expanding T cells in the presence of dasatinib exhibited significantly better tumor killing activity as compared with that treated with Panobinostat or AKTi VIII and the controls, e.g., non-transduced, non-treated, and tumor cells. **p<0.01 by two-way ANOVA with Bonferroni's test for multiple comparisons; compared to Control condition. FIG. 28B shows the quantitative results of that shown in FIG. 29A. ****p<0.0001 by one-way ANOVA with Tukey's test for multiple comparisons; compared to Control condition. (n=3 healthy donors; TCR normalized T cells:UACC257-iRFP=3:1).

FIGS. 29A, 29B, and 29C show T cell products prepared by expanding T cells obtained from three donors (Donor 1, Donor 2, and Donor 3) respectively in the presence of dasatinib exhibited better tumor killing activity as compared with that treated with AKTi VIII and the controls, e.g., non-treated, suggesting that, given variability among different donors, T cell products prepared by dasatinib treatment have consistently higher tumor killing activity than that treated with AKTi VIII. (n=3 healthy donors; TCR normalized T cells:UACC257-RFP=3:1).

TABLE 5

| Criteria | Acceptable range | Panobinostat | SAHA + IL-21 | AKTi VIII | Dasatinib |
|---|---|---|---|---|---|
| Manufacturing | ≥Control | = | −/= | + | = |
| metrics | | | | | |
| (expansion, | | | | | |
| viability, | | | | | |
| yield) | | | | | |
| Memory | Higher % of | +/= | +/= | +/= | ++ |
| Phenotype | Tscm, Tcm, | | | | |
| | lower % of | | | | |
| | TemRA; | | | | |
| | improvements in | | | | |
| | other memory | | | | |
| | markers (CD62L, | | | | |
| | CD28, CD27) | | | | |
| Exhaustion | ≤% of exhausted | = | = | = | + |
| phenotype | cells to control | | | | |
| Tumor killing | ≥Control | + | + | ++ | +++ |
| ability | | | | | |
| Response to | ≥Control | +/= | +/= | +/= | N/A |
| Ag exposure | | | | | |
| (cytokine | | | | | |
| expression, | | | | | |
| exhaustion) | | | | | |

What is claimed is:

1. A method of manufacturing modified T cells comprising:
activating a population of T cells,
transducing the activated T cells with a viral vector,
expanding the transduced T cells,
wherein the activating, the transducing, and/or the expanding is performed in the presence of a histone deacetylase inhibitor (HDACi), an AKT inhibitor (AKTi), or a tyrosine kinase inhibitor (TKi), and
obtaining the expanded T cells.

2. The method of claim 1,
wherein the activating is performed in the presence of the HDACi and the transducing and the expanding are performed in the absence of the HDACi, or
wherein the activating and the transducing are performed in the presence of the HDACi and the expanding is performed in the absence of the HDACi, or
wherein the activating and the expanding are performed in the presence of the HDACi and the transducing is performed in the absence of the HDACi, or
wherein the transducing and the expanding are performed in the presence of the HDACi and the activating is performed in the absence of the HDACi, or
wherein the activating, the transducing, and the expanding are performed in the presence of the HDACi.

3. The method of claim 1, wherein the HDACi is selected from the group consisting of vorinostat (SAHA), belinostat, panobinostat, dacinostat, entinostat, tacedinaline, mocetinostat, and any combination thereof.

4. The method of claim 1, wherein the concentration of the HDACi is from about 0.1 nM to about 5 mM, from about 0.1 nM to about 4 mM, from about 0.1 nM to about 3 mM, from about 0.1 nM to about 2 mM, from about 0.1 nM to about 1 mM, from about 0.1 nM to about 900 nM, from about 0.1 nM to about 800 nM, from about 0.1 nM to about 700 nM, from about 0.1 nM to about 600 nM, from about 0.1 nM to about 500 nM, from about 0.1 nM to about 400 nM, from about 0.1 nM to about 300 nM, from about 0.1 nM to about 200 nM, from about 0.1 nM to about 100 nM, from about 0.1 nM to about 50 nM, from about 0.1 nM to about 40 nM, from about 0.1 nM to about 30 nM, from about 0.1 nM to about 20 nM, from about 0.1 nM to about 10 nM, from about 0.1 nM to about 5 nM, from about 0.1 nM to about 4 nM, from about 0.1 nM to about 3 nM, from about 0.1 nM to about 2 nM, from about 0.1 nM to about 1 nM, from about 0.2 nM to about 1 nM, from about 0.3 nM to about 1 nM, from about 0.4 nM to about 1 nM, from about 0.5 nM to about 1 nM, from about 0.5 nM to about 0.9 nM, from about 0.5 nM to about 0.8 nM, from about 0.5 nM to about 0.7 nM, from about 0.5 nM to about 0.6 nM, from about 1 nM to about 50 nM, or from about 2 nM to about 25 nM.

5. The method of claim 1,
wherein the activating is performed in the presence of the AKTi and the transducing and the expanding are performed in the absence of the AKTi,
wherein the activating and the transducing are performed in the presence of the AKTi and the expanding is performed in the absence of the AKTi, or
wherein the activating and the expanding are performed in the presence of the AKTi and the transducing is performed in the absence of the AKTi, or
wherein the transducing and the expanding are performed in the presence of the AKTi and the activating is performed in the absence of the AKTi, or
wherein the activating, the transducing, and the expanding are performed in the presence of the AKTi.

6. The method of claim 1, wherein the AKTi is selected from the group consisting of (i) 3-[1-[[4-(7-phenyl-3H-imidazo[4, 5g]quinoxalin-6-yl)phenyl]methyl]piperidin-4-yl]-1H-benzimidazol-2-one; (ii) N,N dimethyl-1-[4-(6-phenyl-1H-imidazo[4, 5-g]quinoxalin-7-yl)phenyl]metha-namine; and (iii) I-(I-[4-(3-phenylbenzo[g]quinoxalin-2-yl)benzyl]piperidin-4-yl)-1,-3-dihy-dro-2H benzimidazol-2-one; A6730, B2311, 124018, GSK2110183 (afuresertib), Perifosine (KRX-0401), GDC-0068 (ipatasertib), RX-0201, VQD-002, LY294002, A-443654, A-674563, Akti-1, Akti-2, Akti-1/2, AR-42, API-59CJ-OMe, ATI-13148, AZD-5363, erucylphosphocholine, GSK-2141795 (GSK795), KP372-1, L-418, L-71-101, PBI-05204, PIA5, PX-316, SR13668, triciribine, GSK 690693 (CAS #937174-76-0), FPA 124 (CAS #902779-59-3), Miltefosine, PHT-427 (CAS #1 191951-57-1), 10-DEBC hydrochloride, Akt inhibitor III, MK-2206 dihydrochloride (CAS #1032350-13-2), SC79, AT7867 (CAS #857531-00-1), CCT128930 (CAS #885499-61-6), A-674563 (CAS #552325-73-2), AGL 2263, AS-041 164 (5-benzo[1,3]dioxol-5-ylmethylene-thiazolidine-2,4-dione), BML-257 (CAS #32387-96-5), XL-418, CAS #612847-09-3, CAS #98510-80-6, H-89 (CAS #127243-85-0), OXY-1 1 1 A, 3-[1-[[4-(7-phenyl-3H-imidazo[4,5-g]quinoxalin-6-yl)phenyl]methyl]piperid-in-4-yl]-1H-benzimidazol-2-one, N,N-dimethyl-1-[4-(6-phenyl-1H-imidazo[4,5-g]quinoxalin-7-yl]phenyl]metha-namine, 1-{1-[4-(3-phenylbenzo[g]quinoxalin-2-yl)benzyl]piperidin-4-yl}-1-,-3-dihydro-2H-benzimidazol-2-one, and any combination thereof.

7. The method of claim 1, wherein the concentration of the AKTi is from about 1 nM to about 1 mM, from about 10 nM to about 1 mM, from about 100 nM to about 1 mM, from about 100 nM to about 500 μM, from about 100 nM to about 100 μM, from about 100 nM to about 50 μM, from about 100 nM to about 10 μM, from about 100 nM to about 1 μM, from about 100 nM to about 900 nM, from about 100 nM to about 800 nM, from about 100 nM to about 700 nM, from about 100 nM to about 600 nM, from about 100 nM to about 500 nM, from about 100 nM to about 400 nM, from about 100 nM to about 300 nM, from about 150 nM to about 300 nM, from about 200 nM to about 300 nM, from about 250 nM to about 300 nM, from about 1 μM to about 1 mM, from about 10 μM to about 1 mM, from about 100 μM to about 1 mM, from about 1 nM to about 100 μM, from about 1 nM to about 10 μM, from about 1 nM to about 1 μM, from about 1 nM to about 100 nM, from about 1 nM to about 50 nM, from about 100 nM to about 100 μM, from about 500 nM to about 50 μM, from about 1 μM to about 50 μM, from about 1 μM to about 10 μM, or from about 5 μM to about 10 μM.

8. The method of claim 1,
wherein the activating is performed in the presence of the TKi and the transducing and the expanding are performed in the absence of the TKi, or
wherein the activating and the transducing are performed in the presence of the TKi and the expanding is performed in the absence of the TKi, or
wherein the activating and the expanding are performed in the presence of the TKi and the transducing is performed in the absence of the TKi, or
wherein the transducing and the expanding are performed in the presence of the TKi and the activating is performed in the absence of the TKi, or
wherein the activating, the transducing, and the expanding are performed in the presence of the TKi.

9. The method of claim 1, wherein the TKi is selected from the group consisting of dasatinib, saracatinib, bosutinib, nilotinib, PP1-inhibitor, and any combination thereof.

10. The method of claim 1, wherein the concentration of the TKi is from about 1 nM to about 1 μM, from about 1 nM to about 500 nM, from about 1 nM to about 400 nM, from about 1 nM to about 300 nM, from about 1 nM to about 200 nM, from about 1 nM to about 150 nM, from about 1 nM to about 100 nM, from about 1 nM to about 50 nM, from about 1 nM to about 40 nM, from about 1 nM to about 30 nM, from about 1 nM to about 20 nM, from about 1 nM to about 10 nM, from about 2 nM to about 10 nM, from about 3 nM to about 10 nM, from about 4 nM to about 10 nM, from about 5 nM to about 10 nM, from about 100 nM to about 1 mM, from about 100 nM to about 500 μM, from about 100 nM to about 100 μM, from about 100 nM to about 50 μM, from about 100 nM to about 10 μM, from about 100 nM to about 1 μM, from about 100 nM to about 900 nM, from about 100 nM to about 800 nM, from about 100 nM to about 700 nM, from about 100 nM to about 600 nM, from about 100 nM to about 500 nM, from about 100 nM to about 400 nM, from about 100 nM to about 300 nM, from about 150 nM to about 300 nM, from about 200 nM to about 300 nM, from about 250 nM to about 300 nM, from about 1 μM to about 1 mM, from about 10 μM to about 1 mM, from about 100 μM to about 1 mM, from about 1 nM to about 100 μM, from about 1 nM to about 10 μM, from about 1 nM to about 1 μM, from about 1 nM to about 100 nM, from about 1 nM to about 50 nM, from about 100 nM to about 100 μM, from about 500 nM to about 50 μM, from about 1 μM to about 50 μM, from about 1 μM to about 10 μM, or from about 5 μM to about 10 μM.

11. The method of claim 1, wherein the activating is carried out within a period of from about 1 hour to about 120 hours, about 1 hour to about 108 hours, about 1 hour to about 96 hours, about 1 hour to about 84 hours, about 1 hour to about 72 hours, about 1 hour to about 60 hours, about 1 hour to about 48 hours, about 1 hour to about 36 hours, about 1 hour to about 24 hours, about 2 hours to about 24 hours, about 4 hours to about 24 hours, about 6 hours to about 24 hours, about 8 hours to about 24 hours, about 10 hours to about 24 hours, about 12 hours to about 24 hours, about 12 hours to about 72 hours, about 24 hours to about 72 hours, about 6 hours to about 48 hours, about 24 hours to about 48 hours, about 6 hours to about 72 hours, or about 1 hours to about 12 hours.

12. The method of claim 1, wherein the transducing is carried out within a period of from about 1 hour to 120 hours, about 1 hour to 108 hours, about 1 hour to 96 hours, about 1 hour to 72 hours, about 1 hour to 48 hours, about 1 hour to 36 hours, about 1 hour to 24 hours, about 2 hour to 24 hours, about 4 hour to 24 hours, about 6 hour to 24 hours, about 8 hour to 24 hours, about 10 hour to 24 hours, about 12 hour to 24 hours, about 14 hour to 24 hours, about 16 hour to 24 hours, about 18 hour to 24 hours, about 20 hour to 24 hours, or about 22 hour to 24 hours.

13. The method of claim 1, wherein the expanding is carried out within a period of from about 1 day to about 30 days, about 1 day to about 25 days, about 1 day to about 20 days, about 1 day to about 15 days, about 1 day to about 10 days, about 2 days to about 10 days, about 3 days to about 10 days, about 4 days to about 10 days, about 5 days to about 10 days, about 6 days to about 10 days, about 7 days to about 10 days, about 8 days to about 10 days, or about 9 days to about 10 days.

14. The method of claim 1, wherein the activating, the transducing, and/or the expanding is further performed in the presence of at least one cytokine.

15. The method of claim 14, wherein the at least one cytokine is selected from the group consisting of interleukin (IL)-2, IL-7, IL-12, IL-15, IL-18, and IL-21.

16. A T cell or population of T cells obtained from the method of claim 1.

17. A composition comprising the T cell or population of T cells of claim 16.

18. The composition of claim 17, further comprising an adjuvant selected from the group consisting of an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), and combinations thereof.

19. A method of treating a patient who has cancer, comprising administering to the patient the T cell or population of T cells of claim 16, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

20. A method of eliciting an immune response in a patient who has cancer, comprising administering to the patient T cell or population of T cells of claim 16, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.


SEQUENCE LISTING (294 sequences)

SEQ ID NO: 1 - protein, 114 aa, synthetic construct
  REGION 1..114: note = CD8alpha Ig-like domain-1
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 2 - protein, 38 aa, synthetic construct
  REGION 1..38: note = CD8beta stalk region
SVVDFLPTTAQPTKKSTLKKRVCRLPRPETQKGPLCSP

SEQ ID NO: 3 - protein, 21 aa, synthetic construct
  REGION 1..21: note = CD8alpha transmembrane domain
IYIWAPLAGTCGVLLLSLVIT

SEQ ID NO: 4 - protein, 32 aa, synthetic construct
  REGION 1..32: note = CD8alpha cytoplasmic tail
LYCNHRNRRRVCKCPRPVVKSGDKPSLSARYV

SEQ ID NO: 5 - protein, 205 aa, synthetic construct
  REGION 1..205: note = m1CD8alpha (signal-less)
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 6 - protein, 21 aa, synthetic construct
  REGION 1..21: note = Signal peptide
MALPVTALLLPLALLLHAARP

SEQ ID NO: 7 - protein, 226 aa, synthetic construct
  REGION 1..226: note = m1CD8alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 8 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 9 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 10 - protein, 189 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 11 - protein, 200 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 12 - protein, 225 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 13 - protein, 225 aa, Homo sapiens; identical to SEQ ID NO: 12

SEQ ID NO: 14 - protein, 222 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 15 - protein, 273 aa, synthetic construct
  REGION 1..273: note = R11KEA alpha chain
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 311 aa, synthetic construct
  REGION 1..311: note = R11KE beta chain
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - protein, 274 aa, synthetic construct
  REGION 1..274: note = R20P1H7 alpha chain
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQGENSGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 314 aa, synthetic construct
  REGION 1..314: note = R20P1H7 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGPGLAAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 19 - protein, 272 aa, synthetic construct
  REGION 1..272: note = R7P1D5 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEYSSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 310 aa, synthetic construct
  REGION 1..310: note = R7P1D5 beta chain
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRANTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 21 - protein, 277 aa, synthetic construct
  REGION 1..277: note = R10P2G12 alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEGNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 313 aa, synthetic construct
  REGION 1..313: note = R10P2G12 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLSSGSHQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 271 aa, synthetic construct
  REGION 1..271: note = R10P1A7 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESKETRLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 24 - protein, 317 aa, synthetic construct
  REGION 1..317: note = R10P1A7 beta chain
MLLLLLLLGPGISLLLPGSLAGSGLGAWSQHPSVWICKSGTSVKIECRSLDFQATTMFWYRQFPKQSLMLMATSNEGSKATYEQGVEKDKFLINHASLTLSTLTVTSAHPEDSSFYICSARAGGHEQFFGPGTRLTVLEDLKNVFPPEVAVFEPSEAEISHTQKATLVCLATGFYPDHVELSWVWNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 25 - protein, 271 aa, synthetic construct
  REGION 1..271: note = R4P1D10 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFHDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 308 aa, synthetic construct
  REGION 1..308: note = R4P1D10 beta chain
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSVASAYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 27 - protein, 274 aa, synthetic construct
  REGION 1..274: note = R4P3F9 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 308 aa, synthetic construct
  REGION 1..308: note = R4P3F9 beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVESSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 29 - protein, 271 aa, synthetic construct
  REGION 1..271: note = R4P3H3 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKAGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 314 aa, synthetic construct
  REGION 1..314: note = R4P3H3 beta chain
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLLTSGGDNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 270 aa, synthetic construct
  REGION 1..270: note = R36P3F9 alpha chain
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 314 aa, synthetic construct
  REGION 1..314: note = R36P3F9 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSTSGGLSGETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 33 - protein, 272 aa, synthetic construct
  REGION 1..272: note = R52P2G11 alpha chain
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAYGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - protein, 312 aa, synthetic construct
  REGION 1..312: note = R52P2G11 beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGSPDGNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 35 - protein, 279 aa, synthetic construct
  REGION 1..279: note = R53P2A9 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYNSYAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 310 aa, synthetic construct
  REGION 1..310: note = R53P2A9 beta chain
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSLDGTSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - protein, 271 aa, synthetic construct
  REGION 1..271: note = R26P1A9 alpha chain
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCLIGASGSRLTFGEGTQLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 310 aa, synthetic construct
  REGION 1..310: note = R26P1A9 beta chain
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYFGWNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 39 - protein, 276 aa, synthetic construct
  REGION 1..276: note = R26P2A6 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSDVSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 311 aa, synthetic construct
  REGION 1..311: note = R26P2A6 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASTTPDGTDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 41 - protein, 274 aa, synthetic construct
  REGION 1..274: note = R26P3H1 alpha chain
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDMNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 310 aa, synthetic construct
  REGION 1..310: note = R26P3H1 beta chain
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRAEGGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 43 - protein, 273 aa, synthetic construct
  REGION 1..273: note = R35P3A4 alpha chain
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASPTGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 311 aa, synthetic construct
  REGION 1..311: note = R35P3A4 beta chain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSLGGASQEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 45 - protein, 266 aa, synthetic construct
  REGION 1..266: note = R37P1C9 alpha chain
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 309 aa, synthetic construct
  REGION 1..309: note = R37P1C9 beta chain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSGETNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 277 aa, synthetic construct
  REGION 1..277: note = R37P1H1 alpha chain
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFGYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 48 - protein, 311 aa, synthetic construct
  REGION 1..311: note = R37P1H1 beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSNEGQGWEAEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 49 - protein, 268 aa, synthetic construct
  REGION 1..268: note = R42P3A9 alpha chain
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVHNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 322 aa, synthetic construct
  REGION 1..322: note = R42P3A9 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLLGQGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 51 - protein, 276 aa, synthetic construct
  REGION 1..276: note = R43P3F2 alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSNNNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 52 - protein, 323 aa, synthetic construct
  REGION 1..323: note = R43P3F2 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPTGTSGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 53 - protein, 273 aa, synthetic construct
  REGION 1..273: note = R43P3G5 alpha chain
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 311 aa, synthetic construct
  REGION 1..311: note = R43P3G5 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRLPSRTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 55 - protein, 270 aa, synthetic construct
  REGION 1..270: note = R59P2E7 alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVNSDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 321 aa, synthetic construct
  REGION 1..321: note = R59P2E7 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGLGTGDYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 57 - protein, 273 aa, synthetic construct
  REGION 1..273: note = R11P3D3 alpha chain
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 311 aa, synthetic construct
  REGION 1..311: note = R11P3D3 beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATLLYEILLGKATLYAVLVSALVLMAMVKRKDSRG

SEQ ID NO: 59 - protein, 275 aa, synthetic construct
  REGION 1..275: note = R16P1C10 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 311 aa, synthetic construct
  REGION 1..311: note = R16P1C10 beta chain
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 274 aa, synthetic construct
  REGION 1..274: note = R16P1E8 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 62 - protein, 309 aa, synthetic construct
  REGION 1..309: note = R16P1E8 beta chain
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 63 - protein, 273 aa, synthetic construct
  REGION 1..273: note = R17P1A9 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 314 aa, synthetic construct
  REGION 1..314: note = R17P1A9 beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 65 - protein, 277 aa, synthetic construct
  REGION 1..277: note = R17P1D7 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 313 aa, synthetic construct
  REGION 1..313: note = R17P1D7 beta chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 67 - protein, 273 aa, synthetic construct
  REGION 1..273: note = R17P1G3 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGPSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - protein, 311 aa, synthetic construct
  REGION 1..311: note = R17P1G3 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - protein, 274 aa, synthetic construct
  REGION 1..274: note = R17P2B6 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 70 - protein, 319 aa, synthetic construct
  REGION 1..319: note = R17P2B6 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 71 - protein, 273 aa, synthetic construct; identical to SEQ ID NO: 15
  REGION 1..273: note = R11P3D3KE alpha chain

SEQ ID NO: 72 - protein, 311 aa, synthetic construct; identical to SEQ ID NO: 16
  REGION 1..311: note = R11P3D3KE beta chain

SEQ ID NO: 73 - protein, 273 aa, synthetic construct
  REGION 1..273: note = R39P1C12 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEIDNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 74 - protein, 307 aa, synthetic construct
  REGION 1..307: note = R39P1C12 beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSQLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 75 - protein, 270 aa, synthetic construct
  REGION 1..270: note = R39P1F5 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 76 - protein, 311 aa, synthetic construct
  REGION 1..311: note = R39P1F5 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGANEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 77 - protein, 272 aa, synthetic construct
  REGION 1..272: note = R40P1C2 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 311 aa, synthetic construct
  REGION 1..311: note = R40P1C2 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEMTAVGQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 79 - protein, 271 aa, synthetic construct
  REGION 1..271: note = R41P3E6 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAFSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 80 - protein, 310 aa, synthetic construct
  REGION 1..310: note = R41P3E6 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSQYTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 81 - protein, 270 aa, synthetic construct
  REGION 1..270: note = R43P3G4 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNGGDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - protein, 311 aa, synthetic construct
  REGION 1..311: note = R43P3G4 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGALEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 83 - protein, 281 aa, synthetic construct
  REGION 1..281: note = R44P3B3 alpha chain
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGLYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 84 - protein, 311 aa, synthetic construct
  REGION 1..311: note = R44P3B3 beta chain
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSLGDRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 85 - protein, 272 aa, synthetic construct
  REGION 1..272: note = R44P3E7 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEINNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 320 aa, synthetic construct
  REGION 1..320: note = R44P3E7 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPPDQNTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 87 - protein, 273 aa, synthetic construct
  REGION 1..273: note = R49P2B7 alpha chain
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVRIFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 88 - protein, 312 aa, synthetic construct
  REGION 1..312: note = R49P2B7 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLMGELTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 89 - protein, 276 aa, synthetic construct
  REGION 1..276: note = R55P1G7 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMMGDTGTASKLTFGTGTRLQVTLDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 309 aa, synthetic construct
  REGION 1..309: note = R55P1G7 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFGGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 91 - protein, 270 aa, synthetic construct
  REGION 1..270: note = R59P2A7 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVQPHDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 307 aa, synthetic construct
  REGION 1..307: note = R59P2A7 beta chain
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSGLVAEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 93 - protein, 19 aa, synthetic construct
  REGION 1..19: note = P2A
ATNFSLLKQAGDVEENPGP

SEQ ID NO: 94 - protein, 18 aa, synthetic construct
  REGION 1..18: note = T2A
EGRGSLLTCGDVEENPGP

SEQ ID NO: 95 - protein, 20 aa, synthetic construct
  REGION 1..20: note = E2A
QCTNYALLKLAGDVESNPGP

SEQ ID NO: 96 - protein, 22 aa, synthetic construct
  REGION 1..22: note = F2A
VKQTLNFDLLKLAGDVESNPGP

SEQ ID NO: 97 - protein, 559 aa, synthetic construct
  REGION 1..559: note = RD114TR
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTRLCVQASLLPDIDINPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFNRLVQFVKDRISVVQALVLTQQYHQLKPL

SEQ ID NO: 98 - protein, 10 aa, Homo sapiens
YLYDSETKNA

SEQ ID NO: 99 - protein, 9 aa, Homo sapiens
HLMDQPLSV

SEQ ID NO: 100 - protein, 9 aa, Homo sapiens
GLLKKINSV

SEQ ID NO: 101 - protein, 9 aa, Homo sapiens
FLVDGSSAL

SEQ ID NO: 102 - protein, 10 aa, Homo sapiens
FLFDGSANLV

SEQ ID NO: 103 - protein, 9 aa, Homo sapiens
FLYKIIDEL

SEQ ID NO: 104 - protein, 11 aa, Homo sapiens
FILDSAETTTL

SEQ ID NO: 105 - protein, 9 aa, Homo sapiens
SVDVSPPKV

SEQ ID NO: 106 - protein, 8 aa, Homo sapiens
VADKIHSV

SEQ ID NO: 107 - protein, 9 aa, Homo sapiens
IVDDLTINL

SEQ ID NO: 108 - protein, 9 aa, Homo sapiens
GLLEELVTV

SEQ ID NO: 109 - protein, 10 aa, Homo sapiens
TLDGAAVNQV

SEQ ID NO: 110 - protein, 10 aa, Homo sapiens
SVLEKEIYSI

SEQ ID NO: 111 - protein, 9 aa, Homo sapiens
LLDPKTIFL

SEQ ID NO: 112 - protein, 9 aa, Homo sapiens
YTFSGDVQL

SEQ ID NO: 113 - protein, 9 aa, Homo sapiens
YLMDDFSSL

SEQ ID NO: 114 - protein, 9 aa, Homo sapiens
KVWSDVTPL

SEQ ID NO: 115 - protein, 11 aa, Homo sapiens
LLWGHPRVALA

SEQ ID NO: 116 - protein, 11 aa, Homo sapiens
KIWEELSVLEV

SEQ ID NO: 117 - protein, 9 aa, Homo sapiens
LLIPFTIFM

SEQ ID NO: 118 - protein, 9 aa, Homo sapiens
FLIENLLAA

SEQ ID NO: 119 - protein, 11 aa, Homo sapiens
LLWGHPRVALA

SEQ ID NO: 120 - protein, 9 aa, Homo sapiens
FLLEREQLL

SEQ ID NO: 121 - protein, 9 aa, Homo sapiens
SLAETIFIV

SEQ ID NO: 122 - protein, 9 aa, Homo sapiens
TLLEGISRA

SEQ ID NO: 123 - protein, 9 aa, Homo sapiens
KIQEILTQV

SEQ ID NO: 124 - protein, 10 aa, Homo sapiens
VIFEGEPMYL

SEQ ID NO: 125 - protein, 9 aa, Homo sapiens
SLFESLEYL

SEQ ID NO: 126 - protein, 9 aa, Homo sapiens
SLLNQPKAV

SEQ ID NO: 127 - protein, 9 aa, Homo sapiens
GLAEFQENV

SEQ ID NO: 128 - protein, 9 aa, Homo sapiens
KLLAVIHEL

SEQ ID NO: 129 - protein, 9 aa, Homo sapiens
TLHDQVHLL

SEQ ID NO: 130 - protein, 11 aa, Homo sapiens
TLYNPERTITV

SEQ ID NO: 131 - protein, 9 aa, Homo sapiens
KLQEKIQEL

SEQ ID NO: 132 - protein, 10 aa, Homo sapiens
SVLEKEIYSI

SEQ ID NO: 133 - protein, 11 aa, Homo sapiens
RVIDDSLVVGV

SEQ ID NO: 134 - protein, 9 aa, Homo sapiens
VLFGELPAL

SEQ ID NO: 135 - protein, 9 aa, Homo sapiens
GLVDIMVHL

SEQ ID NO: 136 - protein, 9 aa, Homo sapiens
FLNAIETAL

SEQ ID NO: 137 - protein, 9 aa, Homo sapiens
ALLQALMEL

SEQ ID NO: 138 - protein, 9 aa, Homo sapiens
ALSSSQAEV

SEQ ID NO: 139 - protein, 11 aa, Homo sapiens
SLITGQDLLSV

SEQ ID NO: 140 - protein, 9 aa, Homo sapiens
QLIEKNWLL

SEQ ID NO: 141 - protein, 9 aa, Homo sapiens
LLDPKTIFL

SEQ ID NO: 142 - protein, 9 aa, Homo sapiens
RLHDENILL

SEQ ID NO: 143 - protein, 9 aa, Homo sapiens
YTFSGDVQL

SEQ ID NO: 144 - protein, 9 aa, Homo sapiens
GLPSATTTV

SEQ ID NO: 145 - protein, 11 aa, Homo sapiens
GLLPSAESIKL

SEQ ID NO: 146 - protein, 9 aa, Homo sapiens
KTASINQNV

SEQ ID NO: 147 - protein, 9 aa, Homo sapiens
SLLQHLIGL

SEQ ID NO: 148 - protein, 9 aa, Homo sapiens
YLMDDFSSL

SEQ ID NO: 149 - protein, 9 aa, Homo sapiens
LMYPYIYHV

SEQ ID NO: 150 - protein, 9 aa, Homo sapiens
KVWSDVTPL

SEQ ID NO: 151 - protein, 11 aa, Homo sapiens
LLWGHPRVALA

SEQ ID NO: 152 - protein, 9 aa, Homo sapiens
VLDGKVAVV

SEQ ID NO: 153 - protein, 9 aa, Homo sapiens
GLLGKVTSV

SEQ ID NO: 154 - protein, 9 aa, Homo sapiens
KMISAIPTL

SEQ ID NO: 155 - protein, 11 aa, Homo sapiens
GLLETTGLLAT

SEQ ID NO: 156 - protein, 9 aa, Homo sapiens
TLNTLDINL

SEQ ID NO: 157 - protein, 9 aa, Homo sapiens
VIIKGLEEI

SEQ ID NO: 158 - protein, 9 aa, Homo sapiens
YLEDGFAYV

SEQ ID NO: 159 - protein, 11 aa, Homo sapiens
KIWEELSVLEV

SEQ ID NO: 160 - protein, 9 aa, Homo sapiens
LLIPFTIFM

SEQ ID NO: 161 - protein, 10 aa, Homo sapiens
ISLDEVAVSL

SEQ ID NO: 162 - protein, 10 aa, Homo sapiens
KISDFGLATV

SEQ ID NO: 163 - protein, 11 aa, Homo sapiens
KLIGNIHGNEV

SEQ ID NO: 164 - protein, 9 aa, Homo sapiens
ILLSVLHQL

SEQ ID NO: 165 - protein, 9 aa, Homo sapiens
LDSEALLTL

SEQ ID NO: 166 - protein, 13 aa, Homo sapiens
VLQENSSDYQSNL

SEQ ID NO: 167 - protein, 11 aa, Homo sapiens
HLLGEGAFAQV

SEQ ID NO: 168 - protein, 9 aa, Homo sapiens
SLVENIHVL

SEQ ID NO: 169 - protein, 9 aa, Homo sapiens
YTFSGDVQL

SEQ ID NO: 170 - protein, 9 aa, Homo sapiens
SLSEKSPEV

SEQ ID NO: 171 - protein, 10 aa, Homo sapiens
AMFPDTIPRV

SEQ ID NO: 172 - protein, 9 aa, Homo sapiens
FLIENLLAA

SEQ ID NO: 173 - protein, 9 aa, Homo sapiens
FTAEFLEKV

SEQ ID NO: 174 - protein, 9 aa, Homo sapiens
ALYGNVQQV

SEQ ID NO: 175 - protein, 9 aa, Homo sapiens
LFQSRIAGV

SEQ ID NO: 176 - protein, 11 aa, Homo sapiens
ILAEEPIYIRV

SEQ ID NO: 177 - protein, 9 aa, Homo sapiens
FLLEREQLL

SEQ ID NO: 178 - protein, 10 aa, Homo sapiens
LLLPLELSLA

SEQ ID NO: 179 - protein, 9 aa, Homo sapiens
SLAETIFIV

SEQ ID NO: 180 - protein, 11 aa, Homo sapiens
AILNVDEKNQV

SEQ ID NO: 181 - protein, 9 aa, Homo sapiens
RLFEEVLGV

SEQ ID NO: 182 - protein, 9 aa, Homo sapiens
YLDEVAFML

SEQ ID NO: 183 - protein, 11 aa, Homo sapiens
KLIDEDEPLFL

SEQ ID NO: 184 - protein, 9 aa, Homo sapiens
KLFEKSTGL

SEQ ID NO: 185 - protein, 11 aa, Homo sapiens
SLLEVNEASSV

SEQ ID NO: 186 - protein, 10 aa, Homo sapiens
GVYDGREHTV

SEQ ID NO: 187 - protein, 10 aa, Homo sapiens
GLYPVTLVGV

SEQ ID NO: 188 - protein, 9 aa, Homo sapiens
ALLSSVAEA

SEQ ID NO: 189 - protein, 9 aa, Homo sapiens
TLLEGISRA

SEQ ID NO: 190 - protein, 9 aa, Homo sapiens
SLIEESEEL

SEQ ID NO: 191 - protein, 9 aa, Homo sapiens
ALYVQAPTV

SEQ ID NO: 192 - protein, 10 aa, Homo sapiens
KLIYKDLVSV

SEQ ID NO: 193 - protein, 9 aa, Homo sapiens
ILQDGQFLV

SEQ ID NO: 194 - protein, 9 aa, Homo sapiens
SLLDYEVSI

SEQ ID NO: 195 - protein, 9 aa, Homo sapiens
LLGDSSFFL

SEQ ID NO: 196 - protein, 10 aa, Homo sapiens
VIFEGEPMYL

SEQ ID NO: 197 - protein, 9 aa, Homo sapiens
ALSYILPYL

SEQ ID NO: 198 - protein, 9 aa, Homo sapiens
FLFVDPELV

SEQ ID NO: 199 - protein, 11 aa, Homo sapiens
SEWGSPHAAVP

SEQ ID NO: 200 - protein, 9 aa, Homo sapiens
ALSELERVL

SEQ ID NO: 201 - protein, 9 aa, Homo sapiens
SLFESLEYL

SEQ ID NO: 202 - protein, 9 aa, Homo sapiens
KVLEYVIKV

SEQ ID NO: 203 - protein, 10 aa, Homo sapiens
VLLNEILEQV

SEQ ID NO: 204 - protein, 9 aa, Homo sapiens
SLLNQPKAV

SEQ ID NO: 205 - protein, 9 aa, Homo sapiens
KMSELQTYV

SEQ ID NO: 206 - protein, 11 aa, Homo sapiens
ALLEQTGDMSL

SEQ ID NO: 207 - protein, 11 aa, Homo sapiens
VIIKGLEEITV

SEQ ID NO: 208 - protein, 9 aa, Homo sapiens
KQFEGTVEI

SEQ ID NO: 209 - protein, 9 aa, Homo sapiens
KLQEEIPVL

SEQ ID NO: 210 - protein, 9 aa, Homo sapiens
GLAEFQENV

SEQ ID NO: 211 - protein, 9 aa, Homo sapiens
NVAEIVIHI

SEQ ID NO: 212 - protein, 9 aa, Homo sapiens
ALAGIVTNV

SEQ ID NO: 213 - protein, 12 aa, Homo sapiens
NLLIDDKGTIKL

SEQ ID NO: 214 - protein, 10 aa, Homo sapiens
VLMQDSRLYL

SEQ ID NO: 215 - protein, 9 aa, Homo sapiens
KVLEHVVRV

SEQ ID NO: 216 - protein, 9 aa, Homo sapiens
LLWGNLPEI

SEQ ID NO: 217 - protein, 9 aa, Homo sapiens
SLMEKNQSL

SEQ ID NO: 218 - protein, 9 aa, Homo sapiens
KLLAVIHEL

SEQ ID NO: 219 - protein, 10 aa, Homo sapiens
ALGDKFLLRV

SEQ ID NO: 220 - protein, 11 aa, Homo sapiens
FLMKNSDLYGA

SEQ ID NO: 221 - protein, 10 aa, Homo sapiens
KLIDHQGLYL

SEQ ID NO: 222 - protein, 12 aa, Homo sapiens
GPGIFPPPPPQP

SEQ ID NO: 223 - protein, 9 aa, Homo sapiens
ALNESLVEC

SEQ ID NO: 224 - protein, 9 aa, Homo sapiens
GLAALAVHL

SEQ ID NO: 225 - protein, 9 aa, Homo sapiens
LLLEAVWHL

SEQ ID NO: 226 - protein, 9 aa, Homo sapiens
SIIEYLPTL

SEQ ID NO: 227 - protein, 9 aa, Homo sapiens
TLHDQVHLL

SEQ ID NO: 228 - protein, 9 aa, Homo sapiens
SLLMWITQC

SEQ ID NO: 229 - protein, 11 aa, Homo sapiens
FLLDKPQDLSI

SEQ ID NO: 230 - protein, 10 aa, Homo sapiens
YLLDMPLWYL

SEQ ID NO: 231 - protein, 9 aa, Homo sapiens
GLLDCPIFL

SEQ ID NO: 232 - protein, 9 aa, Homo sapiens
VLIEYNFSI

SEQ ID NO: 233 - protein, 11 aa, Homo sapiens
TLYNPERTITV

SEQ ID NO: 234 - protein, 9 aa, Homo sapiens
AVPPPPSSV

SEQ ID NO: 235 - protein, 9 aa, Homo sapiens
KLQEELNKV

SEQ ID NO: 236 - protein, 11 aa, Homo sapiens
KLMDPGSLPPL

SEQ ID NO: 237 - protein, 9 aa, Homo sapiens
ALIVSLPYL

SEQ ID NO: 238 - protein, 9 aa, Homo sapiens
FLLDGSANV

SEQ ID NO: 239 - protein, 10 aa, Homo sapiens
ALDPSGNQLI

SEQ ID NO: 240 - protein, 9 aa, Homo sapiens
ILIKHLVKV

SEQ ID NO: 241 - protein, 9 aa, Homo sapiens
VLLDTILQL

SEQ ID NO: 242 - protein, 9 aa, Homo sapiens
HLIAEIHTA

SEQ ID NO: 243 - protein, 9 aa, Homo sapiens
SMNGGVFAV

SEQ ID NO: 244 - protein, 9 aa, Homo sapiens
MLAEKLLQA

SEQ ID NO: 245 - protein, 9 aa, Homo sapiens
YMLDIFHEV

SEQ ID NO: 246 - protein, 11 aa, Homo sapiens
ALWLPTDSATV

SEQ ID NO: 247 - protein, 9 aa, Homo sapiens
GLASRILDA

SEQ ID NO: 248 - protein, 9 aa, Homo sapiens
ALSVLRLAL

SEQ ID NO: 249 - protein, 9 aa, Homo sapiens
SYVKVLHHL

SEQ ID NO: 250 - protein, 9 aa, Homo sapiens
VYLPKIPSW

SEQ ID NO: 251 - protein, 9 aa, Homo sapiens
NYEDHFPLL

SEQ ID NO: 252 - protein, 9 aa, Homo sapiens
VYIAELEKI

SEQ ID NO: 253 - protein, 12 aa, Homo sapiens
VHFEDTGKTLLF

SEQ ID NO: 254 - protein, 9 aa, Homo sapiens
VLSPFILTL

SEQ ID NO: 255 - protein, 9 aa, Homo sapiens
HLLEGSVGV

SEQ ID NO: 256 - DNA, 607 bp, synthetic construct
  misc_feature 1..607: note = WPREmut1
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 257 - DNA, 581 bp, synthetic construct
  misc_feature 1..581: note = WPREmut2
  source 1..581: mol_type = other DNA
gagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 258 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 259 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 260 - protein, 47 aa, synthetic construct
  REGION 1..47: note = CD8alpha stalk
KPTTTPAPRPPTPAPTIASQPLSLRPEACRPAAGGAVHTRGLDFACD

SEQ ID NO: 261 - protein, 114 aa, synthetic construct
  REGION 1..114: note = CD8alpha Ig-like domain-2
SQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPA

SEQ ID NO: 262 - protein, 226 aa, synthetic construct
  REGION 1..226: note = m2CD8alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 263 - DNA, 367 bp, synthetic construct
  misc_feature 1..367: note = MSCV promoter
  source 1..367: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcact

SEQ ID NO: 264 - DNA, 607 bp, synthetic construct
  misc_feature 1..607: note = WPRE
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 265 - skipped

SEQ ID NO: 266 - protein, 4 aa, synthetic construct
  REGION 1..4: note = Linker
SGSG

SEQ ID NO: 267 - DNA, 4320 bp, synthetic construct
  misc_feature 1..4320: note = CD8b3.CD8a.TCR.WPREmut2 construct nucleotide sequence
  source 1..4320: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 268 - protein, 1110 aa, synthetic construct
  REGION 1..1110: note = CD8b3.CD8a.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - DNA, 4314 bp, synthetic construct
  misc_feature 1..4314: note = CD8b1(S19).CD8a.TCR.WPREmut2 construct nucleotide sequence
  source 1..4314: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 270 - protein, 1108 aa, synthetic construct
  REGION 1..1108: note = CD8b1(S19).CD8a.TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 271 - DNA, 4428 bp, synthetic construct
  misc_feature 1..4428: note = CD8b4.CD8a.TCR.WPREmut2 construct nucleotide sequence
  source 1..4428: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagaagttcaacatagtctgcctgaagatttccgggttcactacttgctgctgcttccaaatactccagatttcacgagaatatgggttcggtgtgttgctccaaaaagacatcggtcaacgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 272 - protein, 1146 aa, synthetic construct
  REGION 1..1146: note = CD8b4.CD8a.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 273 - DNA, 4308 bp, synthetic construct
  misc_feature 1..4308: note = CD8b1(S19).CD8a(S19).TCR.WPREmut2 construct nucleotide sequence
  source 1..4308: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 274 - protein, 1106 aa, synthetic construct
  REGION 1..1106: note = CD8b1(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 275 - DNA, 4416 bp, synthetic construct
  misc_feature 1..4416: note = CD8b4(S19).CD8a(S19).TCR.WPREmut2 construct nucleotide sequence
  source 1..4416: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagaagttcaacatagtctgcctgaagatttccgggttcactacttgctgctgcttccaaatactccagatttcacgagaatatgggttcggtgtgttgctccaaaaagacatcggtcaacgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 276 - protein, 1142 aa, synthetic construct
  REGION 1..1142: note = CD8b4(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 277 - DNA, 4407 bp, synthetic construct
  misc_feature 1..4407: note = CD8b5(S19).CD8a(S19).TCR.WPREmut2 construct nucleotide sequence
  source 1..4407: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 278 - protein, 1139 aa, synthetic construct
  REGION 1..1139: note = CD8b5(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKTRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 279 - DNA, 3573 bp, synthetic construct
  misc_feature 1..3573: note = CD8a(S19).CD8bstalk.TCR.WPREmut2 construct nucleotide sequence
  source 1..3573: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 280 - protein, 861 aa, synthetic construct
  REGION 1..861: note = CD8a(S19).CD8bstalk.TCR.WPREmut2 amino acid sequence
MEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 281 - DNA, 4284 bp, synthetic construct
  misc_feature 1..4284: note = TCRba.CD8a.CD8b1.2.WPREmut2 construct nucleotide sequence
  source 1..4284: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcccgggccaagagatctggcagcggccagtgcaccaactacgccctgctgaagctggccggcgacgtggagagcaaccccggccccatggcgcttcccgtgaccgcactcctgttgccccttgccctgctgttgcacgccgcacgacctagccagttcagagtgtcccccctggaccggacctggaacctgggagagacagtggaactgaagtgccaggtgctgctgagcaaccccaccagcggctgctcttggctgtttcagcctagaggcgccgctgccagccctacctttctgctgtacctgagccagaacaagcccaaggccgccgagggcctggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgtccctcgtgatcaccctgtactgcaaccaccggaaccggcggagagtgtgcaagtgccctagacccgtcgtgaagtccggcgacaagcctagcctgagcgccagatacgtgcgggccaagagatctggcagcggcgccaccaatttcagcctgctgaaacaggccggcgacgtggaagagaaccctggccccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtctgaaaggtaaggtttaccaagagcctttgtcaccgaacgcttgtatggacacgaccgctatcctccaaccgcataggtcttgtctcactcacgggtcatgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 282 - protein, 1098 aa, synthetic construct
  REGION 1..1098: note = TCRba.CD8a.CD8b1.2.WPREmut2 amino acid sequence
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGATNFSLLKQAGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 283 - DNA, 3606 bp, synthetic construct
  misc_feature 1..3606: note = WT-SPa/CD8a-Ig like domain 1/CD8bstalk/CD4 TM+IC/TCRba/WPREmut2 construct nucleotide sequence
  source 1..3606: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccattcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 284 - protein, 872 aa, synthetic construct
  REGION 1..872: note = WT-SPa/CD8a-Ig like domain 1/CD8bstalk/CD4 TM+IC/TCRba/WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPMALIVLGGVAGLLLFIGLGIFFCVRCRHRRRQAERMSQIKRLLSEKKTCQCPHRFQKTCSPIRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 285 - DNA, 3579 bp, synthetic construct
  misc_feature 1..3579: note = CD8a.CD8bstalk.TCR.WRPEmut2 construct nucleotide sequence
  source 1..3579: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 286 - protein, 863 aa, synthetic construct
  REGION 1..863: note = CD8a.CD8bstalk.TCR.WRPEmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 287 - DNA, 3606 bp, synthetic construct
  misc_feature 1..3606: note = CD8a.TCR.WPREmut2 construct nucleotide sequence
  source 1..3606: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 288 - protein, 872 aa, synthetic construct
  REGION 1..872: note = CD8a.TCR.WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 289 - DNA, 3633 bp, synthetic construct
  misc_feature 1..3633: note = WT-SPa/CD8a-Ig like domain 1(co)/CD8astalk(co)/CD4 TM+IC/TCRba/WPREmut2 construct nucleotide sequence
  source 1..3633: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccattcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 290 - protein, 881 aa, synthetic construct
  REGION 1..881: note = WT-SPa/CD8a-Ig like domain 1(co)/CD8astalk(co)/CD4 TM+IC/TCRba/WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDMALIVLGGVAGLLLFIGLGIFFCVRCRHRRRQAERMSQIKRLLSEKKTCQCPHRFQKTCSPIRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 291 - DNA, 4419 bp, synthetic construct
  misc_feature 1..4419: note = PTE.CD8ab.TCR.WPREmut2 construct nucleotide sequence
  source 1..4419: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 292 - protein, 1143 aa, synthetic construct
  REGION 1..1143: note = PTE.CD8ab.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKTRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 293 - protein, 21 aa, synthetic construct
  REGION 1..21: note = Signal peptide
MRPRLWLLLAAQLTVLHGNSV

SEQ ID NO: 294 - protein, 19 aa, synthetic construct
  REGION 1..19: note = Signal peptide
MEFGLSWLFLVAILKGVQC